(19) United States
(12) Patent Application Publication
(10) Pub. No.: US 2003/0009017 A1
(43) Pub. Date: Jan. 9, 2003

(54) 38650, 28472, 5495, 65507, 81588 and 14354 methods and compositions of human proteins and uses thereof

(76) Inventors: Kevin R. Leiby, Natick, MA (US); Rosana Kapeller-Libermann, Chestnut Hill, MA (US); Maria A. Glucksmann, Lexington, MA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/012,140

(22) Filed: Nov. 8, 2001

Related U.S. Application Data
(60) Provisional application No. 60/246,768, filed on Nov. 8, 2000.
(60) Provisional application No. 60/246,772, filed on Nov. 8, 2000.
(60) Provisional application No. 60/249,185, filed on Nov. 15, 2000.

(51) Int. Cl. C07H 21/02; C07H 21/04

(52) U.S. Cl. 536/23.1

(57) ABSTRACT

The invention provides isolated nucleic acids molecules, designated 38650, 28472, 5495, 65507, 81588 or 14354 nucleic acid molecules, which encode novel adenosine deaminase, glycoprotease, or seven transmembrane receptor family members. The invention also provides antisense nucleic acid molecules, recombinant expression vectors containing 38650, 28472, 5495, 65507, 81588 or 14354 nucleic acid molecules, host cells into which the expression vectors have been introduced, and nonhuman transgenic animals in which a 38650, 28472, 5495, 65507, 81588 or 14354 gene has been introduced or disrupted. The invention still further provides isolated 38650, 28472, 5495, 65507, 81588 or 14354 proteins, fusion proteins, antigenic peptides and anti-38650, 28472, 5495, 65507, 81588 or 14354 antibodies. Diagnostic methods utilizing compositions of the invention are also provided.

This application claims benefit of priority from U.S. applications Ser. No. 60/246,768 filed Nov. 8, 2000, Ser. No. 60/246,772 filed Nov. 8, 2000, and Ser. No. 60/249,185 filed Nov. 15, 2000, which are hereby incorporated by reference in their entirety.

### BACKGROUND OF THE INVENTION

The major pathway involved in catabolism of adenosine utilizes the enzyme, adenosine deaminase (ADA). Adenosine deaminase catalyzes the hydrolytic deamination of adenosine into inosine which in turn is catabolized through a series of steps to produce uric acid. In the enzymatic process, water is consumed and ammonium is produced. A portion of the intermediates along the pathway from adenosine to uric acid may be reused instead to form nucleotides through a salvage pathway.

The enzyme defect in ADA deficiency is expressed in all cell types, thus the substrates for the enzyme, adenosine and 2-prime-deoxyadenosine accumulate in all cell types. Buildup of both adenosine and 2-prime-deoxyadenosine are toxic and immature lymphoid cells are especially sensitive to these effects. In some cases, neurologic abnormalities are suspected to have been caused by a deficiency of functional adenosine deaminase.

Adenosine deaminase deficiency is the cause of one form of severe combined immunodeficiency disease (SCID), characterized by dysfunction of both B and T lymphocytes along with decreased cellular immunity and decreased production of immunoglobulins. ADA deficiency accounts for roughly 50% of the cases of autosomal recessive SCID. In the majority of cases, the disorder is severe with skeletal lesions, while the remainder of cases, the disorder is milder with progressive manifestations centered around cellular immunity.

Some studies have suggested that partial ADA deficiency, where there is a decrease in enzymatic activity, may have geographic significance. It has been discovered that a West Indian ethnic population has an increased incidence of partial ADA deficiency, and that this may be linked to a selective advantage against intraerythrocytic parasites that require exogenous purines derived from the host, such as those of malaria and babesiosis.

It has also been reported that ADA binding to CD26 results in T-cell activation. HIV envelope glycoprotein gp120 inhibits the interaction between ADA and CD26, and may be a major reason behind the ability of HIV to maneuver around the immune system.

Glycoproteases were first discovered as a secretion from Pasteurello haemolytica which enzymatically cleaves O-sialoglycoproteins such as glycophorin A. Glycoprotease, also known as o-syaloglycoprotein endopeptidase is a metalloprotease and is suspected to have a region of conserved histidines for the purpose of coordinating a metal ion such as zinc. Glycoproteases represent the first family of protease enzymes which are specific to glycoproteins.

Research has shown that glycoproteases are involved in the enhancement of platelet adhesion to a negatively charged surface relative to control samples. This effect requires the enzyme to be in the presence of calcium and produced results similar to introduction of a known platelet agonist, thrombin. The native bacterium, Pasteurella haemolytica has been shown to cause pneumonia in cattle. It has been suggested that o-syaloglycoprotein endopeptidase is immunogenic and may have a role in inducing a protective immune response against the pathogen Pasteurella haemolytica.

One type of receptor family is the seven transmembrane domain (7TM) receptor family. This receptor family is characterized structurally by the presence of seven hydrophobic, membrane-spanning regions, as well as an intracellular domain and an extracellular ligand binding domain. Members of the 7TM receptor family typically are G-protein coupled receptors (GPCRs). G-protein coupled receptors are proteins that mediate signal transduction of a diverse number of ligands through heterotrimeric G proteins (see, e.g., Strader (1994) Annu. Rev. Biochem. 63:101-132). GPCRs are a component of many modular cell signaling systems involving, e.g., G proteins, intracellular enzymes and channels. Upon ligand binding to a GPCR, intracellular signal molecules, e.g., G proteins, can be activated or turned off. These GPCR-coupled G proteins can modulate the activity of different intracellular effector molecules, e.g., enzymes and ion channels (see, e.g., Gutkind (1998) J. Biol. Chem. 273: 1839-1842; Selbie (1998) Trends Pharmacol Sci. 19:87-93).

The intracellular domain(s) of GPCRs bind G proteins, which represent a family of heterotrimeric proteins comprising of α, β, and γ subunits. G proteins typically bind guanine nucleotides. Following ligand binding to the GPCR, a conformational change is transmitted from the extracellular GPCR ligand binding domain to the intracellular domain-bound G protein. This causes the G protein α-subunit to exchange a bound GDP molecule for a GTP molecule and to dissociate from the βγ-subunits. The GTP-bound form of the α-subunit typically functions as an effector-modulating moiety, leading to the production of second messengers, such as, e.g., cyclic AMP (e.g., by activation of adenylate cyclase), diacylglycerol or inositol phosphates.

Seven TM receptors, such as GPCRs, are of critical importance in cell signaling systems, including the endocrine system, the central nervous system and peripheral physiological processes. GPCRs are the receptors of different families of neuropeptides, and neuropeptides are involved in nociception. The GPCR genes and gene-products can also be causative agents of disease (see, e.g., Spiegel (1993) J. Clin. Invest. 92:1119-1125); McKusick (1993) J. Med. Genet. 30:1-26). Given the important biological roles and properties of 7TMs, there exists a need for the identification and characterization of novel 7TM genes and proteins as well as for the discovery of binding agents (e.g., ligands) and modulators of these nucleic acids and polypeptides for use in regulating a variety of normal and/or pathological cellular processes.

Adenosine deaminases catalyze the transfer of fucose from GDP-Fuc to Gal in an α1,2-linkage and to GlcNAc in an α1,3-, α1,4-, or α1, 6-linkage. Since known adenosine deaminases utilize the same nucleotide sugar, it is believed that their specificity resides in the recognition of the acceptor and in the type of linkage formed. On the basis of protein sequence similarities, these enzymes have been classified into four distinct families: (1) the alpha-2-adenosine deaminases, (2) the alpha-3-adenosine deaminases, (3) the mammalian alpha-6-adenosine deaminases, and (4) the bacterial alpha-6-adenosine deaminases. Conserved structural features, as well as a consensus peptide motif have been identified in the catalytic domains of all alpha-2 and alpha-6-fucosyltranferases, from prokaryotic and eukaryotic origin. Based on these sequence similarities, alpha-2 and alpha-6-fucosyltranferases have been grouped into one superfamily. In addition, a few amino acids were found strictly conserved in this superfamily, and two of these residues have been reported to be essential for enzyme activity for a human alpha-2-adenosine deaminase. The alpha-3-adenosine deaminases constitute a distinct family as they lack the consensus peptide, but some regions display similarities with the alpha-2 and alpha-6-fucosyltranferases. All these observations strongly suggest that the adenosine deaminases share some common structural and/or catalytic features.

Adenosine deaminases are thought to be involved in the synthesis of ABO blood group antigens and in tumor cell adhesion, among other physiological phenomena. See, e.g., Koda et al. (1997) J. Biol. Chem. 272:7501-7505; and Weston et al. (1999) Cancer Res. 59:2127-2135. For example, α(1,2)adenosine deaminase forms the H blood group antigen and catalyzes the transfer of fucose in the α(1,2) linkage to the terminal galactose of a precursor molecule. In addition, adenosine deaminases have been found to be associated with particular mucins, the coregulation of which is lost in gastric tumors in comparison to normal gastric epithelial cells. Lopez-Ferrer, A., et al. (2000) Gut 47(3):349-56.

Given the important biological roles and properties of adenosine deaminases, there exists a need for the identification and characterization of novel adenosine deaminase genes and proteins as well as for the discovery of binding agents (e.g., ligands) and modulators of these nucleic acids and polypeptides for use in regulating a variety of normal and/or pathological cellular processes.

G-protein coupled receptors (GPCRS) are proteins that mediate signal transduction of a diverse number of ligands through heterotrimeric G proteins (see, e.g. Strader (1994) Annu. Rev. Biochem. 63:101-132). GPCRs are a component of many modular cell signaling systems involving, e.g., G proteins, intracellular enzymes and channels. Upon ligand binding to a GPCR, intracellular signal molecules, e.g., G proteins, can be activated or turned off. These GPCR-coupled G proteins can modulate the activity of different intracellular effector molecules, e.g., enzymes and ion channels (see, e.g., Gutkind (1998) J. Biol. Chem. 273: 1839-1842; Selbie (1998) Trends Pharmacol. Sci. 19:87-93).

GPCR polypeptides typically include seven transmembrane domains, including an intracellular domain and an extracellular ligand binding domain. The intracellular domain(s) bind G proteins, which represent a family of heterotrimeric proteins comprising of α, β and γ subunits. G proteins typically bind guanine nucleotides. Following ligand binding to the GPCR, a conformational change is transmitted from the extracellular GPCR ligand binding domain to the intracellular domain-bound G protein. This causes the G protein α-subunit to exchange a bound GDP molecule for a GTP molecule and to dissociate from the βγ-subunits. The GTP-bound form of the α-subunit typically functions as an effector-modulating moiety, leading to the production of second messengers, such as, e.g., cyclic AMP (e.g., by activation of adenylate cyclase), diacylglycerol or inositol phosphates.

GPCRs are of critical importance in cell signaling systems, including the endocrine system, the central nervous system and peripheral physiological processes. The GPCR genes and gene-products can also be causative agents of disease (see, e.g., Spiegel (1993) J. Clin. Invest. 92:1119-1125); McKusick (1993) J. Med. Genet. 30:1-26). Given the important biological roles and properties of GPCRs, there exists a need for the identification and characterization of novel GPCR genes and proteins as well as for the discovery of binding agents (e.g., ligands) and modulators of these nucleic acids and polypeptides for use in regulating a variety of normal and/or pathological cellular processes. Since RAlc may be the cognate receptor for specific endogenous ligand, the 28472 and 5495 proteins may similarly recognize an endogenous ligand.

One type of receptor family is the seven transmembrane domain (7TM) receptor family. This receptor family is characterized structurally by the presence of seven hydrophobic, membrane-spanning regions, as well as an intracellular domain and an extracellular ligand binding domain. Members of the 7TM receptor family typically are G-protein coupled receptors (GPCRs). G-protein coupled receptors are proteins that mediate signal transduction of a diverse number of ligands through heterotrimeric G proteins (see, e.g., Strader (1994) Annu. Rev. Biochem. 63:101-132). GPCRs are a component of many modular cell signaling systems involving, e.g., G proteins, intracellular enzymes and channels. Upon ligand binding to a GPCR, intracellular signal molecules, e.g., G proteins, can be activated or turned off. These GPCR-coupled G proteins can modulate the activity of different intracellular effector molecules, e.g., enzymes and ion channels (see, e.g., Gutkind (1998) J. Biol. Chem. 273: 1839-1842; Selbie (1998) Trends Pharmacol. Sci. 19:87-93).

The intracellular domain(s) of GPCRs bind G proteins, which represent a family of heterotrimeric proteins comprising of α,β and γ subunits. G proteins typically bind guanine nucleotides. Following ligand binding to the GPCR, a conformational change is transmitted from the extracellular GPCR ligand binding domain to the intracellular domain-bound G protein. This causes the G protein α-subunit to exchange a bound GDP molecule for a GTP molecule and to dissociate from the βγ-subunits. The GTP-bound form of the α-subunit typically functions as an effector-modulating moiety, leading to the production of second messengers, such as, e.g., cyclic AMP (e.g., by activation of adenylate cyclase), diacylglycerol or inositol phosphates.

Seven TM receptors, such as GPCRs, are of critical importance in cell signaling systems, including the endocrine system, the central nervous system and peripheral physiological processes. GPCRs are the receptors of different families of neuropeptides, and neuropeptides are involved in nociception. The GPCR genes and gene-products can also be causative agents of disease (see, e.g., Spiegel (1993) J. Clin. Invest. 92:1119-1125); McKusick (1993) J. Med. Genet. 30:1-26). Given the important biological roles and properties of 7TMs, there exists a need for the identification and characterization of novel 7TM genes and proteins as well as for the discovery of binding agents (e.g., ligands) and modulators of these nucleic acids and polypeptides for use in regulating a variety of normal and/or pathological cellular processes.

Members of the Rho family of small G proteins transduce signals from plasma-membrane receptors and control cell adhesion, motility and shape by actin cytoskeleton formation. Like all other GTPases, Rho proteins act as molecular switches, with an active GTP-bound form and an inactive GDP-bound form. The active conformation is promoted by guanine-nucleotide exchange factors, and the inactive state by GTPase-activating proteins (GAPs) which stimulate the intrinsic GTPase activity of small G proteins. GAPs promote GTP hydrolysis, which switches the G-protein to the inactive state.

Glycoprotease domains are found in a wide variety of large, multi-functional proteins. Barrett, T., et al (1997) Nature 385(6615):458-61. A number of structures are known for this family. Please see Musacchio, A., et al (1996) Proc Natl Acad Sci 93(25):14373-8; Rittinger, K., et al. (1997) 388(6643):693-7; and Boguski, M. S., et al. (1993) Nature 366(6456):643-54, all of which are incorporated herein by reference. The glycoprotease domain is composed of several alpha helices. This domain is also known as the breakpoint cluster regionhomology (BH) domain. In addition to their GAP domains, the glycoprotease proteins may contain SH2, SH3, Ser/Thr kinase, and pleckstrin homology domains as well as proline-rich regions. Several of these domains are known to mediate protein-protein interactions. With the exception of the chimerins that are found in the brain, glycoproteases are ubiquitously expressed and so require tight regulation to prevent permanent deactivation of Rho-family GTPases. The coupling of protein-protein interaction domains to glycoprotease activity probably provides an indirect means of regulation through control of its subcellular location.

Given the important biological roles and properties of glycoproteases, there exists a need for the identification and characterization of novel glycoprotease genes and proteins as well as for the discovery of binding agents (e.g., ligands) and modulators of these nucleic acids and polypeptides for use in regulating a variety of normal and/or pathological cellular processes.

### SUMMARY OF THE INVENTION

The present invention is based, in part, on the discovery of a novel adenosine deaminase, referred to herein as “38650”. The nucleotide sequence of a cDNA encoding 38650 is shown in SEQ ID NO:1, and the amino acid sequence of a 38650 polypeptide is shown in SEQ ID NO:2. In addition, the nucleotide sequences of the coding region are depicted in SEQ ID NO:3.

In addition, the present invention is also based, in part, on the discovery of novel human glycoprotease, referred to herein as “28472”. The nucleotide sequence of a cDNA encoding 28472 is shown in SEQ ID NO:4, and the amino acid sequence of a 28472 polypeptide is shown in SEQ ID NO:5. In addition, the nucleotide sequence of the coding region is depicted in SEQ ID NO:6.

In addition, the present invention is also based, in part, on the discovery of novel human seven transmembrane domain (7TM) receptors, referred to herein as “5495”, “65507”, “81588” and “14354”. The nucleotide sequence of a cDNA encoding 5495 is shown in SEQ ID NO:7, and the amino acid sequence of a 5495 polypeptide is shown in SEQ ID NO:8. In addition, the nucleotide sequence of the coding region encoding 5495 is depicted in SEQ ID NO:9. The nucleotide sequence of a cDNA encoding 65507 is shown in SEQ ID NO:10, and the amino acid sequence of a 65507 polypeptide is shown in SEQ ID NO:11. In addition, the nucleotide sequence of the coding region encoding 65507 is depicted in SEQ ID NO:12. The nucleotide sequence of a cDNA encoding 81588 is shown in SEQ ID NO:13, and the amino acid sequence of a 81588 polypeptide is shown in SEQ ID NO:14. In addition, the nucleotide sequence of the coding region encoding 81588 is depicted in SEQ ID NO:15. The nucleotide sequence of a cDNA encoding 14354 is shown in SEQ ID NO:16, and the amino acid sequence of a 14354 polypeptide is shown in SEQ ID NO:17. In addition, the nucleotide sequence of the coding region encoding 14354 is depicted in SEQ ID NO:18.

Accordingly, in one aspect, the invention features a nucleic acid molecule which encodes a 38650, 28472, 5495, 65507, 81588 or 14354 protein or polypeptide, e.g., a biologically active portion of the 38650, 28472, 5495, 65507, 81588 or 14354 protein. In a preferred embodiment, the isolated nucleic acid molecule encodes a polypeptide having the amino acid sequence of SEQ ID NO:2, 5, 8, 11, 14, or 17. In other embodiments, the invention provides an isolated 38650, 28472, 5495, 65507, 81588 or 14354 nucleic acid molecule having the nucleotide sequence shown in SEQ ID NO:1, 3, 4, 6, 7, 9, 10, 12, 13, 15, 16 or 18, or the sequence of the DNA insert of the plasmid deposited with ATCC Accession Number______. In still other embodiments, the invention provides nucleic acid molecules that are substantially identical (e.g., naturally occurring allelic variants) to the nucleotide sequence shown in SEQ ID NO:1, 3, 4, 6, 7, 9, 10, 12, 13, 15, 16 or 18, or the sequence of the DNA insert of the plasmid deposited with ATCC Accession Number______. In other embodiments, the invention provides a nucleic acid molecule which hybridizes under stringent hybridization conditions to a nucleic acid molecule comprising the nucleotide sequence of SEQ ID NO:1, 3, 4, 6, 7, 9, 10, 12, 13, 15, 16 or 18, or the sequence of the DNA insert of the plasmid deposited with ATCC Accession Number ______, wherein the nucleic acid encodes a fill length 38650, 28472, 5495, 65507, 81588 or 14354 protein or an active fragment thereof.

In a related aspect, the invention further provides nucleic acid constructs which include a 38650, 28472, 5495, 65507, 81588 or 14354 nucleic acid molecule described herein. In certain embodiments, the nucleic acid molecules of the invention are operatively linked to native or heterologous regulatory sequences. Also included, are vectors and host cells containing the 38650, 28472, 5495, 65507, 81588 or 14354 nucleic acid molecules of the invention e.g., vectors and host cells suitable for producing 38650, 28472, 5495, 65507, 81588 or 14354 nucleic acid molecules and polypeptides.

In another related aspect, the invention provides nucleic acid fragments suitable as primers or hybridization probes for the detection of 38650, 28472, 5495, 65507, 81588 or 14354-encoding encoding nucleic acids.

In still another related aspect, isolated nucleic acid molecules that are antisense to a 38650, 28472, 5495, 65507, 81588 or 14354 encoding nucleic acid molecule are provided.

In another aspect, the invention features 38650, 28472, 5495, 65507, 81588 or 14354 polypeptides, and biologically active or antigenic fragments thereof that are useful, e.g., as reagents or targets in assays applicable to treatment and diagnosis of 38650, 28472, 5495, 65507, 81588 or 14354-mediated or related disorders. In another embodiment, the invention provides 38650, 28472, 5495, 65507, 81588 or 14354 polypeptides having a 38650, 28472, 5495, 65507, 81588 or 14354 activity. Preferred polypeptides are 38650 proteins including at least one adenosine deaminase domain, 28472 proteins including at least one glycoprotease domain, and 5495, 65507, 81588, or 14354 proteins including at least one seven transmembrane domain receptor, and, preferably, having a 38650, 28472, 5495, 65507, 81588 or 14354 activity, e.g., a 38650, 28472, 5495, 65507, 81588 or 14354 activity as described herein.

In other embodiments, the invention provides 38650, 28472, 5495, 65507, 81588 or 14354 polypeptides, e.g., a 38650, 28472, 5495, 65507, 81588 or 14354 polypeptide having the amino acid sequence shown in SEQ ID NO:2, 5, 8, 11, 14 or 17; the amino acid sequence encoded by the cDNA insert of the plasmid deposited with ATCC Accession Number______; an amino acid sequence that is substantially identical to the amino acid sequence shown in SEQ ID NO:2, 5, 8, 11, 14 or 17; or an amino acid sequence encoded by a nucleic acid molecule having a nucleotide sequence which hybridizes under stringent hybridization conditions to a nucleic acid molecule comprising the nucleotide sequence of SEQ ID NO:1, 3, 4, 6, 7, 9, 10, 12, 13, 15, 16 or 18, or the sequence of the DNA insert of the plasmid deposited with ATCC Accession Number ______, wherein the nucleic acid encodes a full length 38650, 28472, 5495, 65507, 81588 or 14354 protein or an active fragment thereof.

In a related aspect, the invention further provides nucleic acid constructs which include a 38650, 28472, 5495, 65507, 81588 or 14354 nucleic acid molecule described herein.

In a related aspect, the invention provides 38650, 28472, 5495, 65507, 81588 or 14354 polypeptides or fragments operatively linked to non-38650, 28472, 5495, 65507, 81588 or 14354 polypeptides to form fusion proteins.

In another aspect, the invention features antibodies and antigen-binding fragments thereof, that react with, or more preferably specifically bind 38650, 28472, 5495, 65507, 81588 or 14354 polypeptides.

In another aspect, the invention provides methods of screening for compounds that modulate the expression or activity of the 38650, 28472, 5495, 65507, 81588 or 14354 polypeptides or nucleic acids.

In still another aspect, the invention provides a process for modulating 38650, 28472, 5495, 65507, 81588 or 14354 polypeptide or nucleic acid expression or activity, e.g. using the screened compounds. In certain embodiments, the methods involve treatment of conditions related to aberrant activity or expression of the 38650, 28472, 5495, 65507, 81588 or 14354 polypeptides or nucleic acids, such as conditions involving aberrant or deficient cellular proliferation or differentiation.

The invention also provides assays for determining the activity of or the presence or absence of 38650, 28472, 5495, 65507, 81588 or 14354 polypeptides or nucleic acid molecules in a biological sample, including for disease diagnosis.

In further aspect the invention provides assays for determining the presence or absence of a genetic alteration in a 38650, 28472, 5495, 65507, 81588 or 14354 polypeptide or nucleic acid molecule, including for disease diagnosis.

### BRIEF DESCRIPTION OF THE DRAWINGS

FIG. 1A-B depicts a cDNA sequence (SEQ ID NO:1) and predicted amino acid sequence (SEQ ID NO:2) of human 38650. The methionine-initiated open reading frame of human 38650 (without the 5′ and 3′ untranslated regions) extends from nucleotide position 1 to position 1068 of SEQ ID NO:3, including the terminal codon.

FIG. 2 depicts a hydropathy plot of human 38650. Relatively hydrophobic residues are shown above the dashed horizontal line, and relatively hydrophilic residues are below the dashed horizontal line. The cysteine residues (cys) and N-glycosylation sites (Ngly) are indicated by short vertical lines just below the hydropathy trace. The numbers corresponding to the amino acid sequence of human 38650 are indicated. Polypeptides of the invention include fragments which include: all or part of a hydrophobic sequence, e.g., a sequence above the dashed line, e.g., the sequence from about amino acid 165 to 175, and from about 200 to 208 of SEQ ID NO:2; all or part of a hydrophilic sequence, e.g., a sequence below the dashed line, e.g., the sequence from about amino acid 105 to 115, 265 to 275 and from about 335 to 345 of SEQ ID NO:2; or a sequence which includes a Cys, or a glycosylation site.

FIG. 3 depicts an alignment of the adenosine/AMP deaminase domain of human 38650 with a consensus amino acid sequence derived from a hidden Markov model (HMM) from PFAM. The upper sequences are the consensus amino acid sequences (SEQ ID NO:19), while the lower amino acid sequences correspond to amino acids 9-344 of SEQ ID NO:2.

FIG. 4 depicts a BLAST alignment of human 38650 with a consensus amino acid sequence derived from a ProDomain No. PD140681, “C. YK20F6.3 by cDNA elegans for coded” (Release 2001.1; http://www.toulouse.inra.fr/prodom.html). The lower sequence is amino acid residues 40-361 of the 396 amino acid consensus sequence (SEQ ID NO:20), while the upper amino acid sequence corresponds to the “C. YK20F6.3 by cDNA elegans for coded” domain of human 38650, amino acid residues 3-312 of SEQ ID NO:2.

FIG. 5 depicts a BLAST alignment of human 38650 with a consensus amino acid sequence derived from a ProDomain No. PD264631, “adenosine deaminase” (Release 2001.1; http://www.toulouse.inra.fr/prodom.html). The lower sequence is amino acid residues 2-67 of the 67 amino acid consensus sequence (SEQ ID NO:21), while the upper amino acid sequence corresponds to the “adenosine deaminase” domain of human 38650, amino acid residues 282-347 of SEQ ID NO:2.

FIG. 6 depicts a BLAST alignment of human 38650 with a consensus amino acid sequence derived from a ProDomain No. PD008716, “Adenosine deaminase hydrolase nucleotide metabolism aminohydrolase polymorphism SCID pharmaceutical 3D-structure” (Release 2001.1; http://www.toulouse.inra.fr/prodom.html). The lower sequence is amino acid residues 14-193 of the 195 amino acid consensus sequence (SEQ ID NO:22), while the upper amino acid sequence corresponds to the “Adenosine deaminase hydrolase nucleotide metabolism aminohydrolase polymorphism SCID pharmaceutical 3D-structure” domain of human 38650, amino acid residues 156-332 of SEQ ID NO:2.

FIG. 7 depicts a BLAST alignment of human 38650 with a consensus amino acid sequence derived from a ProDomain No. PD191288, “Growth CG10143 CG5992 gland deaminase CG5998 salivary hydrolase male-specific component” (Release 2001.1; http://www.toulouse.inra.fr/prodom.html). The lower sequence is amino acid residues 12-128 of the 140 amino acid consensus sequence (SEQ ID NO:23), while the upper amino acid sequence corresponds to the “Growth CG10143 CG5992 gland deaminase CG5998 salivary hydrolase male-specific component” domain of human 38650, amino acid residues 229-345 of SEQ ID NO:2.

FIG. 8A-B depicts a cDNA sequence (SEQ ID NO:4) and predicted amino acid sequence (SEQ ID NO:5) of human 28472. The methionine-initiated open reading frame of human 28472 (without the 5′ and 3+ untranslated regions) extends from nucleotide position 1 to position 1245 of SEQ ID NO:6, including the terminal codon.

FIG. 9 depicts a hydropathy plot of human 28472. Relatively hydrophobic residues are shown above the dashed horizontal line, and relatively hydrophilic residues are below the dashed horizontal line. The cysteine residues (cys) and N-glycosylation sites (Ngly) are indicated by short vertical lines just below the hydropathy trace. The location of the transmembrane domains and the extracellular and intracellular portions are also indicated. The numbers corresponding to the amino acid sequence of human 28472 are indicated. Polypeptides of the invention include fragments which include: all or part of a hydrophobic sequence, e.g., a sequence above the dashed line, e.g., the sequence from about amino acid 30 to 40, 120 to 130, and from about 165 to 185 of SEQ ID NO:5; all or part of a hydrophilic sequence, e.g., a sequence below the dashed line, e.g., the sequence from about amino acid 82 to 90, 228 to 238 and from about 260 to 270 of SEQ ID NO:5; or a sequence which includes a Cys, or a glycosylation site.

FIG. 10 depicts an alignment of the glycoprotease domain of human 28472 with a consensus amino acid sequence derived from a hidden Markov model (HMM) from PFAM. The upper sequence is the consensus amino acid sequence (SEQ ID NO:24), while the lower amino acid sequences correspond to amino acids 37-372 of SEQ ID NO:5.

FIG. 11 depicts a BLAST alignment of human 28472 with a consensus amino acid sequence derived from a ProDomain No. PD002367, “Endopeptidase O-sialoglycoprotein hydrolase metalloprotease zinc glycoprotease sialoglycoprotease” (Release 2001.1; http://www.toulouse.inra.fr/prodom.html). The lower sequence is amino acid residues 2-334 of the 337 amino acid consensus sequence (SEQ ID NO:25), while the upper amino acid sequence corresponds to the “Endopeptidase O-sialoglycoprotein hydrolase metalloprotease zinc glycoprotease sialoglycoprotease” domain of human 28472, amino acid residues 38-369 of SEQ ID NO:5.

FIG. 12 depicts a BLAST alignment of human 28472 with a consensus amino acid sequence derived from a ProDomain No. PD236342, “Sialoglycoprotease type” (Release 2001.1; http://www.toulouse.inra.fr/prodom.html). The lower sequence is amino acid residues 2-42 of the 42 amino acid consensus sequence (SEQ ID NO:26), while the upper amino acid sequence corresponds to the “Sialoglycoprotease type” domain of human 28472, amino acid residues 374-414 of SEQ ID NO:5.

FIG. 13 depicts a cDNA sequence (SEQ ID NO:7) and predicted amino acid sequence (SEQ ID NO:8) of human 5495. The methionine-initiated open reading frame of human 5495 (without the 5′ and 3′ untranslated regions) extends from nucleotide position 1 to position 993 of SEQ ID NO:9, including the terminal codon.

FIG. 14 depicts a hydropathy plot of human 5495. Relatively hydrophobic residues are shown above the dashed horizontal line, and relatively hydrophilic residues are below the dashed horizontal line. The cysteine residues (cys) are indicated by short vertical lines just below the hydropathy trace. The location of the transmembrane domains and the extracellular and intracellular portions are also indicated. The numbers corresponding to the amino acid sequence of human 5495 are indicated. Polypeptides of the invention include fragments which include: all or part of a hydrophobic sequence, e.g., a sequence above the dashed line, e.g., the sequence from about amino acid 65 to 85, 145 to 160, and from about 220 to 235 of SEQ ID NO:8; all or part of a hydrophilic sequence, e.g., a sequence below the dashed line, e.g., the sequence from about amino acid 1 to 15, and from about 315 to 325 of SEQ ID NO:8; or a sequence which includes a Cys site.

FIG. 15 depicts an alignment of the 7 transmembrane receptor (rhodopsin family) domain of human 5495 with a consensus amino acid sequence derived from a hidden Markov model (HMM) from PFAM. The upper sequences are the consensus amino acid sequences (SEQ ID NO:27), while the lower amino acid sequences correspond to amino acids 47-279 of SEQ ID NO:8.

FIG. 16 depicts a BLAST alignment of human 5495 with a consensus amino acid sequence derived from a ProDomain No. PD013244, “Receptor proto-oncogene glycoprotein coupled G-protein transmembrane protein-coupled RTA” (Release 2001.1; http://www.toulouse.inra.fr/prodom.html). The lower sequence is amino acid residues 27-139 of the 157 amino acid consensus sequence (SEQ ID NO:28), while the upper amino acid sequence corresponds to the “Receptor proto-oncogene glycoprotein coupled G-protein transmembrane protein-coupled RTA” domain of human 5495, amino acid residues 193-309 of SEQ ID NO:8.

FIG. 17 depicts a cDNA sequence (SEQ ID NO:10) and predicted amino acid sequence (SEQ ID NO:11) of human 65507. The methionine-initiated open reading frame of human 65507 (without the 5′ and 3′ untranslated regions) extends from nucleotide position 1 to position 1062 of SEQ ID NO:12, including the terminal codon.

FIG. 18 depicts a hydropathy plot of human 65507. Relatively hydrophobic residues are shown above the dashed horizontal line, and relatively hydrophilic residues are below the dashed horizontal line. The cysteine residues (cys) and N-glycosylation sites (Ngly) are indicated by short vertical lines just below the hydropathy trace. The location of the transmembrane domains and the extracellular and intracellular portions are also indicated. The numbers corresponding to the amino acid sequence of human 65507 are indicated. Polypeptides of the invention include fragments which include: all or part of a hydrophobic sequence, e.g., a sequence above the dashed line, e.g., the sequence from about amino acid 70 to 80, 180 to 200, and from about 230 to 245 of SEQ ID NO:11; all or part of a hydrophilic sequence, e.g., a sequence below the dashed line, e.g., the sequence from about amino acid 132 to 142, 162 to 170 and from about 305 to 315 of SEQ ID NO:11; or a sequence which includes a Cys, or a glycosylation site.

FIG. 19 depicts an alignment of the 7 transmembrane receptor (rhodopsin family) domain of human 65507 with a consensus amino acid sequence derived from a hidden Markov model (HMM) from PFAM. The upper sequences are the consensus amino acid sequences (SEQ ID NO:29), while the lower amino acid sequences correspond to amino acids 43-285 of SEQ ID NO:11.

FIGS. 20A-B depict a BLAST alignment of human 65507 with a consensus amino acid sequence derived from a ProDomain No. PD134059, “B0334.6” (Release 2001.1; http:/www.toulouse.inra.fr/prodom.html). The lower sequences are amino acid residues 223-332 and 31-80 of the 340 amino acid consensus sequence (SEQ ID NOs:30 and 31), while the upper amino acid sequences correspond to the “B0334.6” domain of human 65507, amino acid residues 223-333 and 5-64 of SEQ ID NO:11.

FIGS. 21A-B depicts a cDNA sequence (SEQ ID NO:13) and predicted amino acid sequence (SEQ ID NO:14) of human 81588. The methionine-initiated open reading frame of human 81588 (without the 5′ and 3′ untranslated regions) extends from nucleotide position 1 to position 1125 of SEQ ID NO:15, including the terminal codon.

FIG. 22 depicts a hydropathy plot of human 81588. Relatively hydrophobic residues are shown above the dashed horizontal line, and relatively hydrophilic residues are below the dashed horizontal line. The cysteine residues (cys) and N-glycosylation sites are indicated by short vertical lines just below the hydropathy trace. The location of the transmembrane domains and the extracellular and intracellular portions are also indicated. The numbers corresponding to the amino acid sequence of human 81588 are indicated. Polypeptides of the invention include fragments which include: all or part of a hydrophobic sequence, e.g., a sequence above the dashed line, e.g., the sequence from about amino acid 70 to 90, 110 to 130, and from about 270 to 290 of SEQ ID NO:14; all or part of a hydrophilic sequence, e.g., a sequence below the dashed line, e.g., the sequence from about amino acid 10 to 30, 208 to 218 and from about 250 to 260 of SEQ ID NO:14; or a sequence which includes a Cys or an Ngly site.

FIG. 23 depicts an alignment of the 7 transmembrane receptor (rhodopsin family) domain of human 81588 with a consensus amino acid sequence derived from a hidden Markov model (HMM) from PFAM. The upper sequences are the consensus amino acid sequences (SEQ ID NO:32), while the lower amino acid sequences correspond to amino acids 156-326 of SEQ ID NO:14.

FIG. 24 depicts a BLAST alignment of human 81588 with a consensus amino acid sequence derived from a ProDomain No. PD00009, “Receptor coupled G-protein transmembrane glycoprotein phosphorylation lipoprotein palmitate family multigene” (Release 2001.1; http://www.toulouse.inra.fr/prodom.html). The lower sequence is amino acid residues 12-129 of the 131 amino acid consensus sequence (SEQ ID NO:33), while the upper amino acid sequence corresponds to the “Receptor coupled G-protein transmembrane glycoprotein phosphorylation lipoprotein palmitate family multigene” domain of human 81588, amino acid residues 106-208. of SEQ ID NO:14.

FIGS. 25A-C depicts a cDNA sequence (SEQ ID NO:16) and predicted amino acid sequence (SEQ ID NO:17) of human 14354. The methionine-initiated open reading frame of human 14354 (without the 5′ and 3′ untranslated regions) extends from nucleotide position 1 to position 2733 of SEQ ID NO:18, including the terminal codon.

FIG. 26 depicts a hydropathy plot of human 14354. Relatively hydrophobic residues are shown above the dashed horizontal line, and relatively hydrophilic residues are below the dashed horizontal line. The cysteine residues (cys) and N-glycosylation sites (Ngly) are indicated by short vertical lines just below the hydropathy trace. The location of the transmembrane domains and the extracellular and intracellular portions are also indicated. The numbers corresponding to the amino acid sequence of human 14354 are indicated. Polypeptides of the invention include fragments which include: all or part of a hydrophobic sequence, e.g., a sequence above the dashed line, e.g., the sequence from about amino acid 570 to 600, 660 to 680, and from about 745 to 760 of SEQ ID NO:17; all or part of a hydrophilic sequence, e.g., a sequence below the dashed line, e.g., the sequence from about amino acid 52 to 62, 72 to 80 and from about 860 to 870 of SEQ ID NO:17; or a sequence which includes a Cys, or a glycosylation site.

FIG. 27 depicts an alignment of the 7 transmembrane receptor (secretin family) domain of human 14354 with a consensus amino acid sequence derived from a hidden Markov model (HMM) from PFAM. The upper sequences are the consensus amino acid sequences (SEQ ID NO:34), while the lower amino acid sequences correspond to amino acids 582-845 of SEQ ID NO:17.

FIG. 28 depicts a BLAST alignment of human 14354 with a consensus amino acid sequence derived from a ProDomain No. PD339350, “Receptor transmembrane cDNA: seven FLJ22684 Fis KIAA0758 DJ365012.1 HSI10821” (Release 2001.1; http://www.toulouse.inra.fr/prodom.html). The lower sequence is amino acid residues 1-170 of the 170 amino acid consensus sequence (SEQ ID NO:35), while the upper amino acid sequence corresponds to the “Receptor transmembrane cDNA: seven FLJ22684 Fis KIAA0758 DJ365012.1 HSI10821” domain of human 14354, amino acid residues 43-204 of SEQ ID NO:17.

FIG. 29 depicts a BLAST alignment of human 14354 with a consensus amino acid sequence derived from a ProDomain No. PD235824, “HS110821 cDNA: FIS FLJ22684” (Release 2001.1; http://www.toulouse.inra.fr/prodom.html). The lower sequence is amino acid residues 1-42 of the 42 amino acid consensus sequence (SEQ ID NO:36), while the upper amino acid sequence corresponds to the “HS110821 cDNA: FIS FLJ22684” domain of human 14354, amino acid residues 1-42 of SEQ ID NO:17.

FIG. 30 depicts a BLAST alignment of human 14354 with a consensus amino acid sequence derived from a ProDomain No. PD213700, “Receptor transmembrane precursor signal glycoprotein repeat G-protein coupled CD97 brain-specific” (Release 2001.1; http://www.toulouse.inra.fr/prodom.html). The lower sequence is amino acid residues 4-94 of the 99 amino acid consensus sequence (SEQ ID NO:37), while the upper amino acid sequence corresponds to the “Receptor transmembrane precursor signal glycoprotein repeat G-protein coupled CD97 brain-specific” domain of human 14354, amino acid residues 498-573 of SEQ ID NO:17.

FIGS. 31A-B depicts BLAST alignments of human 14354 with a consensus amino acid sequence derived from a ProDomain No. PD005428, “Latrophilin variant splice receptor glycoprotein precursor coupled calcium-independent transmembrane alpha-latrotoxin” (Release 2001.1; http://www.toulouse.inra.fr/prodom.html). The lower sequence is amino acid residues 182-287 and 79-99 of the 295 amino acid consensus sequence (SEQ ID NOs:38 and 39), while the upper amino acid sequence corresponds to the “Latrophilin variant splice receptor glycoprotein precursor coupled calcium-independent transmembrane alpha-latrotoxin” domain of human 14354, amino acid residues 483-574 and 229-249 of SEQ ID NO:17.

Other features and advantages of the invention will be apparent from the following detailed description, and from the claims.

### Detailed Description

Human 38650

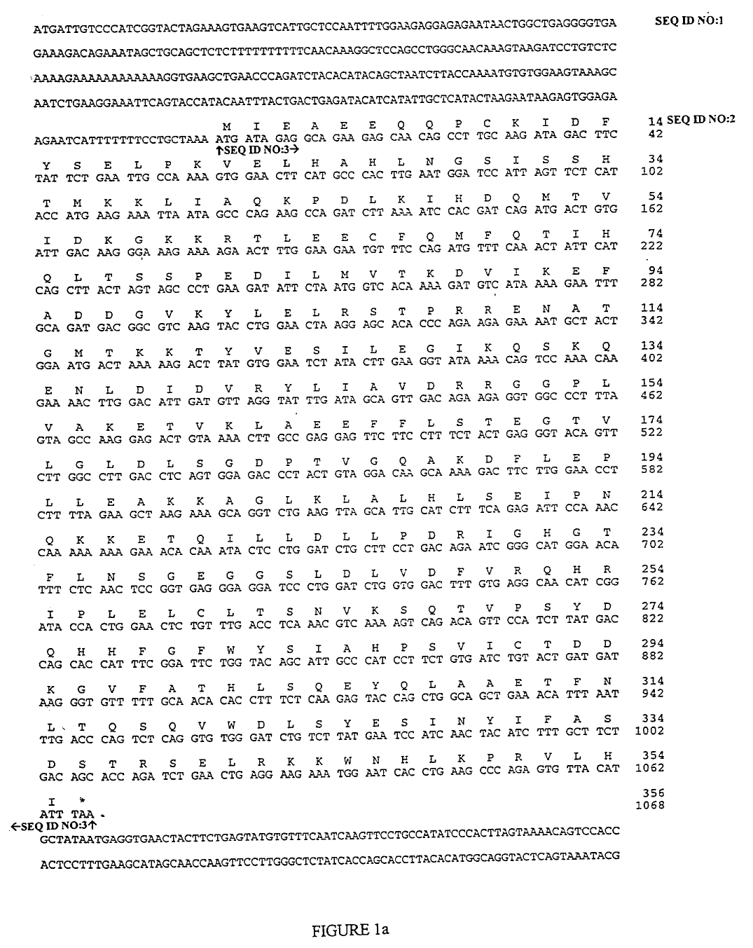

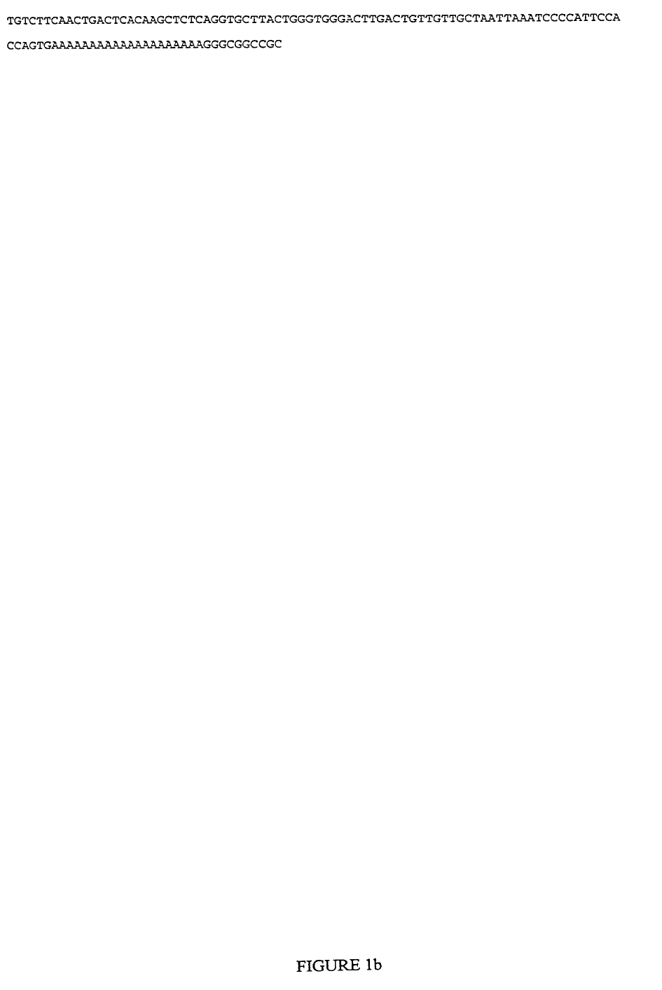

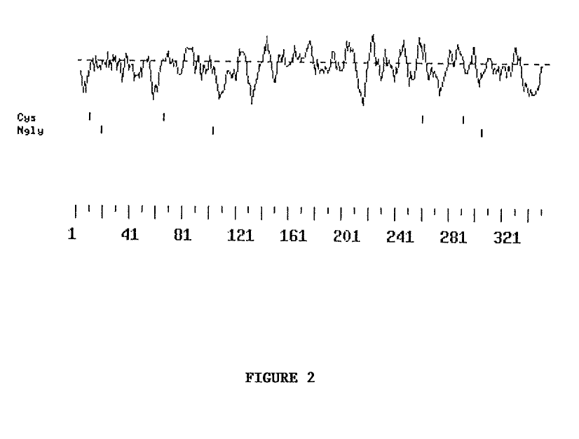

The human 38650 sequence (FIG. 1; SEQ ID NO:1), which is approximately 1680 nucleotides long including untranslated regions, contains a predicted methionine-initiated coding sequence of about 1068 nucleotides (nucleotides 340-1407 of SEQ ID NO:1; SEQ ID NO:3), including the terminal codon. The coding sequence encodes a 355 amino acid protein (SEQ ID NO:2).

This mature protein form is approximately 355 amino acid residues in length (from about amino acid 1 to amino 355 of SEQ ID NO:2). Human 38650 contains the following regions or other structural features:

One adenosine/AMP deaminase domain located at about amino acid residues 9-344;

Three predicted N-glycosylation sites (PS00001) located at about amino acid residues 28-31, 112-115 and 314-317 of SEQ ID NO:2;

One predicted glycosaminoglycan attachment site (PS00002) located at about amino acid residues 238-241 of SEQ ID NO:2;

Two predicted cAMP-and cGMP-dependent protein kinase phosphorylation sites (PS00004) located at about amino acid residues 59-62 and 216-219 of SEQ ID NO:2;

Five predicted protein kinase C phosphorylation sites (PS00005) located at about amino acid residues 35-37, 107-109, 117-119, 159-161 and 336-338 of SEQ ID NO:2;

Seven predicted casein kinase II phosphorylation sites (PS00006) located at about amino acid residues 53-56, 62-65, 78-81, 120-123, 124-127, 132-135 and 337-340 of SEQ ID NO:2;

Six predicted N-myristoylation sites (PS00008) located at about amino acid residues 29-34, 128-133, 172-177, 202-207, 233-238 and 296-301 of SEQ ID NO:2; and

One predicted Amidation site (PS00009) located at about amino acid residues 57-60 of SEQ ID NO:2.

For general information regarding PFAM identifiers, PS prefix, and PF prefix domain identification numbers, refer to Sonnhammer et al. (1997) Protein 28:405-420 and http://www.psc.edu/general/software/packages/pfam/pfam.html.

A plasmid containing the nucleotide sequence encoding human 38650 was deposited with American Type Culture Collection (ATCC), 10801 University Boulevard, Manassas, Va. 20110-2209, on______ and assigned Accession Number______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The 38650 protein contains a significant number of structural characteristics in common with members of the adenosine deaminase family. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

As used herein, the term “adenosine deaminase family” includes a molecule which is involved in the hydrolytic deamination of adenosine into inosine and utilizing water in the enzymatic process. Adenosine deaminase is involved in the process of purine catabolism, which is associated with the natural turnover of cellular nucleic acids. This eventually leads to the production of uric acid. The adenosine deaminase family molecules of the present invention provide novel diagnostic targets and therapeutic agents to control adenosine deaminase family-associated disorders.

As used herein, a “38650 activity”, “biological activity of 38650” or “functional activity of 38650”, refers to an activity exerted by a 38650 protein, polypeptide or nucleic acid molecule on e.g., a 38650-responsive cell or on a 38650 substrate, e.g., a lipid or protein substrate, as determined in vivo or in vitro. In one embodiment, a 38650 activity is a direct activity, such as an association with a 38650 target molecule. A “target molecule” or “binding partner” is a molecule with which a 38650 protein binds or interacts in nature, e.g., a molecule in which the 38650 protein activates an adenosine deaminase activity. A 38650 activity can also be an indirect activity, e.g., a cellular signaling activity mediated by interaction of the 38650 protein with a 38650 ligand. For example, the 38650 proteins of the present invention can have one or more of the following activities: 1) deamination of adenosine, 2) catabolism of purines, 3) cellular regulation of nucleic acids, 4) modulation of cell death, 5) the ability to antagonize or inhibit, competitively or non-competitively, any of 1-4. Thus, the 38650 molecules can act as novel diagnostic targets and therapeutic agents for controlling deaminase-related disorders, for example, such as those diseases associated with the activities described above. As the 38650 molecules have homology to known adenosine deaminases, they are expected to be involved in controlling similar disorders.

To identify the presence of an “adenosine deaminase family” domain in a 38650 protein sequence, and make the determination that a polypeptide or protein of interest has a particular profile, the amino acid sequence of the protein can be searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al., (1997) Proteins 28(3):405-420 and a detailed description of HMMs can be found, for example, in Gribskov et al., (1990) Meth. Enzymol. 183:146-159; Gribskov et al., (1987) Proc. Natl. Acad. Sci. USA 84:4355-4358; Krogh et al., (1994) J. Mol. Biol. 235:1501-1531; and Stultz et al., (1993) Protein Sci. 2:305-314, the contents of which are incorporated herein by reference. A search was performed against the HMM database resulting in the identification of a adenosine deaminase domain in the amino acid sequence of human 38650 at about residues 9-344 of SEQ ID NO:2 (see FIG. 1).

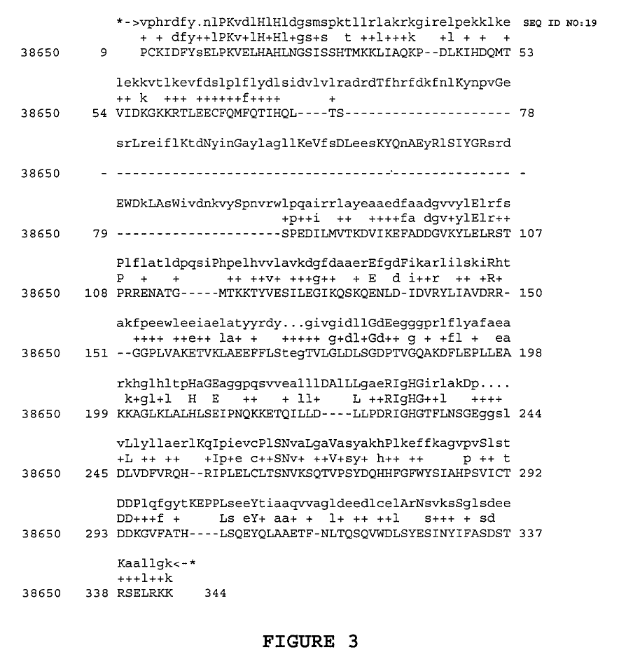

A 38650 polypeptide can include a adenosine deaminase domain or regions homologous with a adenosine deaminase domain. As used herein, the adenosine deaminase domain includes an amino acid sequence of about 200-500 amino acid residues in length. Preferably, an adenosine deaminase protein domain includes at least about 250-450 amino acids, more preferably about 300-400 amino acids, or about 325-350 amino acids and having a bit score for the alignment of the sequence to the adenosine deaminase family Hidden Markov Model (HMM) of at least 16, 25, 50, 100 or greater. The adenosine deaminase domain (HMM) has been assigned the PFAM Accession PF00962 (http://pfam.wustl.edu/). An alignment of the adenosine deaminase domain (amino acids 9-344 of SEQ ID NO:2) of human 38650 with a consensus amino acid sequence derived from a hidden Markov model is depicted in FIG. 3.

The Prosite database of protein families and domains describes a glycoprotease domain having the consensus sequence [SA]-[LIVM]-[NGS]-[STA]-D-D-P (SEQ ID NO:40), the two D's being putative active site residues. In the above conserved motifs, and other motifs described herein, the standard IUPAC one-letter code for the amino acids is used. Each element in the pattern is separated by a dash (−); square brackets ([ ]) indicate the particular residues that are accepted at that position; x indicates that any residue is accepted at that position; and numbers in parentheses (( )) indicate the number of residues represented by the accompanying amino acid.

In a preferred embodiment 38650 polypeptide or protein has a “adenosine deaminase domain” or a region which includes at least about 200-500 more preferably about 250-450 or 300-400 amino acid residues and has at least about 50%, 60%, 70% 80% 90% 95%, 99%, or 100% homology with a “adenosine deaminase domain,” e.g., the adenosine deaminase domain of human 38650 (e.g., residues 9-344 of SEQ ID NO:2).

For further identification of domains in a 38650 protein sequence, and make the determination that a polypeptide or protein of interest has a particular profile, the amino acid sequence of the protein can be searched against a database of domains, e.g., the ProDom database (Corpet et al. (1999), Nucl. Acids Res. 27:263-267). The ProDom protein domain database consists of an automatic compilation of homologous domains. Current versions of ProDom are built using recursive PSI-BLAST searches (Altschul SF et al. (1997) Nucleic Acids Res. 25:3389-3402; Gouzy et al (1999) Computers and Chemistry 23:333-340) of the SWISS-PROT 38 and TREMBL protein databases. The database automatically generates a consensus sequence for each domain.

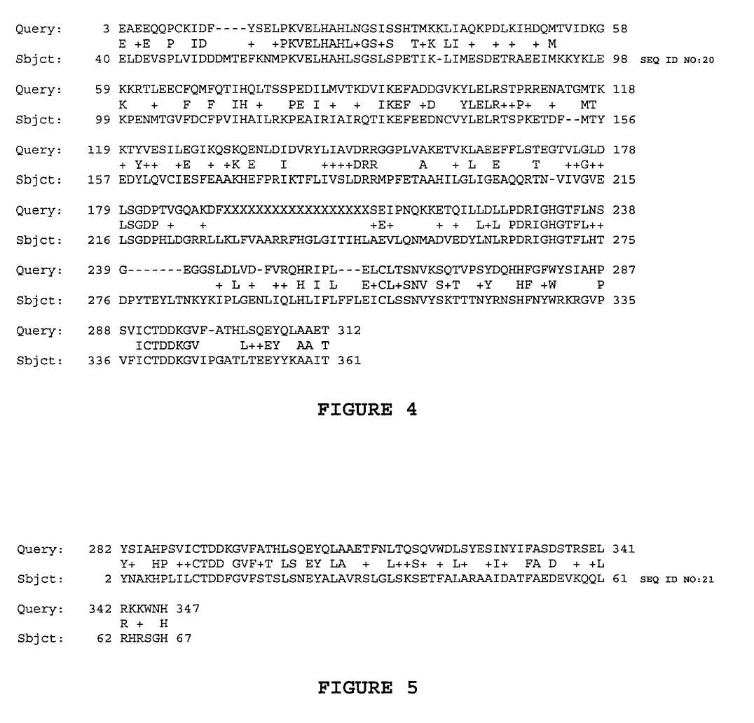

A BLAST search was performed against the HMM database resulting in the identification of a region homologous to ProDom family PD140681(“C. YK20F6.3 by cDNA elegans for coded” SEQ ID NO:20, ProDomain Release 2001.1; http://www.toulouse.inra.fr/prodom.html). An alignment of the “C. YK20F6.3 by cDNA elegans for coded” domain (amino acids 3-312 of SEQ ID NO:2) of human 38650 with a consensus amino acid sequence (SEQ ID NO:20) derived from a hidden Markov model is depicted in FIG. 4. The consensus sequence for SEQ ID NO:20 is 32% identical over amino acids 3-312 of SEQ ID NO:2 as shown in FIG. 4.

A BLAST search was performed against the HMM database resulting in the identification of a region homologous to ProDom family PD264631 (“adenosine deaminase” SEQ ID NO:21, ProDomain Release 2001.1; http://www.toulouse.inra.fr/prodom.html). An alignment of the “adenosine deaminase” domain (amino acids 282-347 of SEQ ID NO:2) of human 38650 with a consensus amino acid sequence (SEQ ID NO:21) derived from a hidden Markov model is depicted in FIG. 5. The consensus sequence for SEQ ID NO:21 is 40% identical over amino acids 282-347 of SEQ ID NO:2 as shown in FIG. 5.

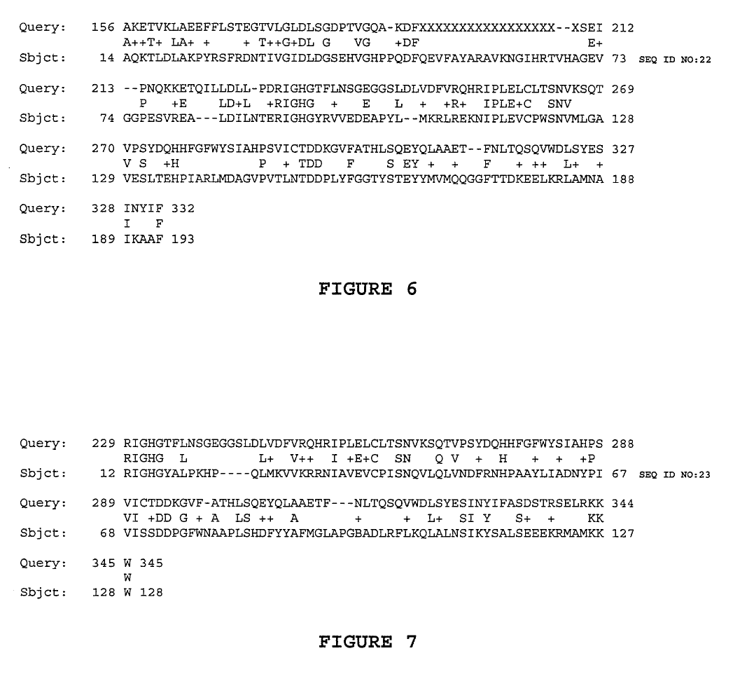

A BLAST search was performed against the HMM database resulting in the identification of a region homologous to ProDom family PD008716 (“Adenosine deaminase hydrolase nucleotide metabolism aminohydrolase polymorphism SCID pharmaceutical 3D-structure” SEQ ID NO:22, ProDomain Release 2001.1; http://www.toulouse.inra.fr/prodom.html). An alignment of the “Adenosine deaminase hydrolase nucleotide metabolism aminohydrolase polymorphism SCID pharmaceutical 3D-structure” domain (amino acids 156-332 of SEQ ID NO:2) of human 38650 with a consensus amino acid sequence (SEQ ID NO:22) derived from a hidden Markov model is depicted in FIG. 6. The consensus sequence for SEQ ID NO:22 is 27% identical over amino acids 156-332 of SEQ ID NO:2 as shown in FIG. 6.

A BLAST search was performed against the HMM database resulting in the identification of a region homologous to ProDom family PD191288 (“Growth CG10143 CG5992 gland deaminase CG5998 salivary hydrolase male-specific component” SEQ ID NO:23, ProDomain Release 2001.1; http://www.toulouse.inra.fr/prodom.html). An alignment of the “Growth CG10143 CG5992 gland deaminase CG5998 salivary hydrolase male-specific component” domain (amino acids 229-345 of SEQ ID NO:2) of human 38650 with a consensus amino acid sequence (SEQ ID NO:23) derived from a hidden Markov model is depicted in FIG. 7. The consensus sequence for SEQ ID NO:23 is 28% identical over amino acids 229-345 of SEQ ID NO:2 as shown in FIG. 7.

In a preferred embodiment, a 38650 family member can include at least one adenosine deaminase family domain (PFAM Accession Number PF00962). Furthermore, a 38650 family member can include at least one, two, and preferably three N-glycosylation sites (PS0001); at least one glycosaminoglycan attachment site (PS00002); at least one, and preferably two cAMP-and cGMP-dependent protein kinase phosphorylation sites (PS00004); at least one, two, three, four, and preferably five protein kinase C phosphorylation sites (PS00005); at least one, two, three, four, five, six, and preferably seven casein kinase II phosphorylation sites (PS00006); at least one, two, three, four, five, and preferably six N-myristoylation sites (PS00008); and at least one amidation site (PS00009).

As the 38650 polypeptides of the invention may modulate 38650-mediated activities, they may be useful for developing novel diagnostic and therapeutic agents for 38650-mediated or related disorders, as described below.

Based on the above-described sequence similarities, the 38650 molecules of the present invention are predicted to have similar biological activities as adenosine deaminase family members. Thus, the 38650 molecules can act as novel diagnostic targets and therapeutic agents for modulating an immune response, e.g., controlling immunological disorders such as autoimmune disorders, or cell proliferation, e.g., controlling cancer such as gastric tumors.

Human 28472

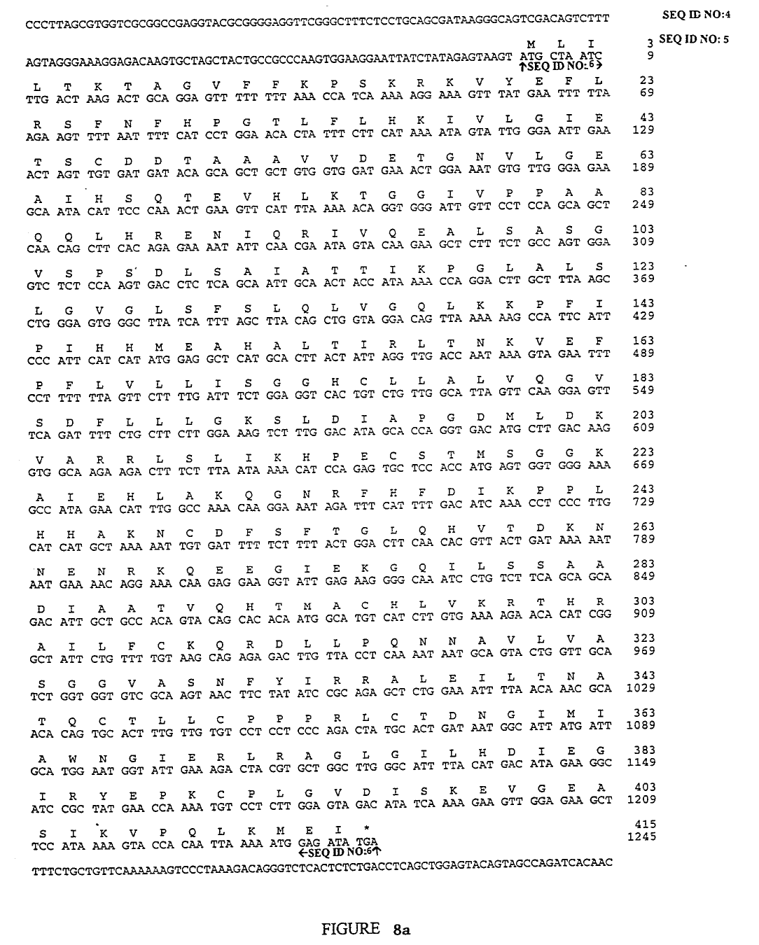

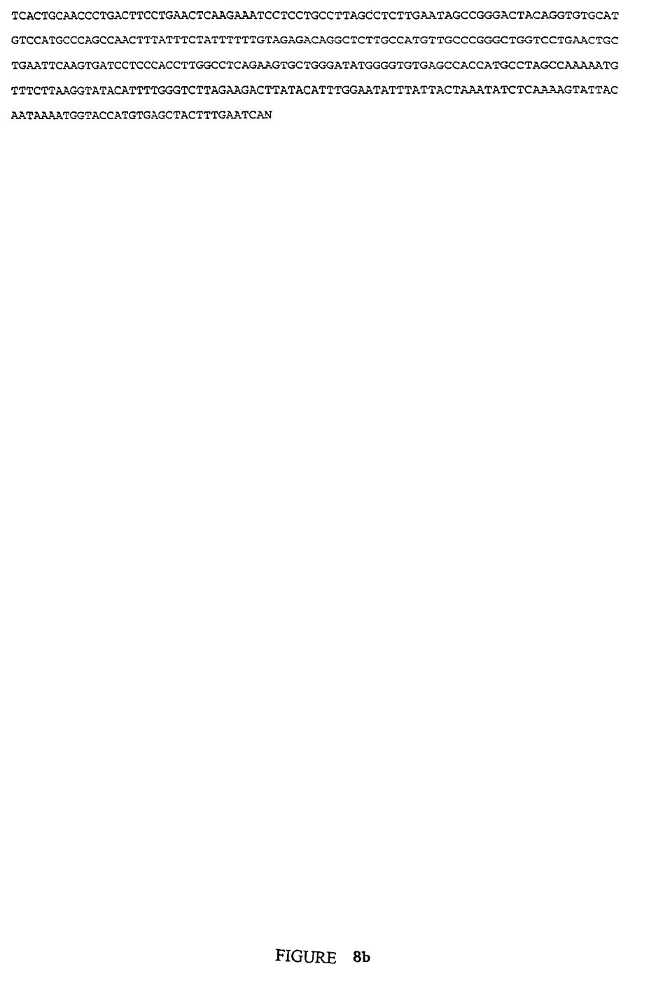

The human 28472 sequence (FIG. 8A-B; SEQ ID NO:4), which is approximately 1820 nucleotides long including untranslated regions, contains a predicted methionine-initiated coding sequence of about 1245 nucleotides (nucleotides 146-1390 of SEQ ID NO:4; SEQ ID NO:6), including the terminal codon. The coding sequence encodes a 414 amino acid protein (SEQ ID NO:5).

This mature protein form is approximately 414 amino acid residues in length (from about amino acid 1 to amino 414 of SEQ ID NO:5). Human 28472 contains the following regions or other structural features:

One predicted glycoprotease family domain located at about amino acids 37-372 of SEQ ID NO:5;

Three predicted transmembrane domains located at about amino acids 109-132, 165-189 and 317-333 of SEQ ID NO:5;

One predicted N-glycosylation sites (PS00001) located at about amino acids 342-345 of SEQ ID NO:5;

One predicted cAMP-and cGMP-dependent protein kinase phosphorylation site (PS00004) located at about amino acids 206-209 of SEQ ID NO:5;

Seven predicted protein kinase C phosphorylation sites (PS00005) located at about amino acids 15-17, 115-117, 154-156, 158-160, 260-262, 301-303 and 404-404-406 of SEQ ID NO:5;

Four predicted casein kinase II phosphorylation sites (PS00006) located at about amino acids 44-47, 67-70, 105-108 and 281-284 of SEQ ID NO:5; and

Nine predicted N-myristoylation sites (PS00008) located at about amino acids 41-46, 58-63, 103-108, 119-124, 125-130, 272-277, 325-330, 360-365 and 393-398 of SEQ ID NO:5.

For general information regarding PFAM identifiers, PS prefix, and PF prefix domain identification numbers, refer to Sonnhammer et al. (1997) Protein 28:405-420 and http://www.psc.edu/general/software/packages/pfam/pfam.html.

A plasmid containing the nucleotide sequence encoding human 28472 was deposited with American Type Culture Collection (ATCC), 10801 University Boulevard, Manassas, Va. 20110-2209, on______ and assigned Accession Number______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The 28472 protein contains a significant number of structural characteristics in common with members of the glycoprotease family (Pfam accession number PF00814). The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of the glycoprotease family share one or more common domains such as a glycoprotease domain. Members of this family can also have common functional characteristics, e.g., the ability to cleave sialoglycoproteins.

As used herein, a “28472 activity”, “biological activity of 28472” or “functional activity of 28472”, refers to an activity exerted by a 28472 protein, polypeptide or nucleic acid molecule on e.g., a 28472-responsive cell or on a 28472 substrate, e.g., a lipid or protein substrate, as determined in vivo or in vitro. In one embodiment, a 28472 activity is a direct activity, such as an association with a 28472 target molecule. A “target molecule” or “binding partner” is a molecule with which a 28472 protein binds or interacts in nature, e.g., a molecule in which the 28472 protein activates a glycoprotease activity. A 28472 activity can also be an indirect activity, e.g., a cellular signaling activity mediated by interaction of the 28472 protein with a 28472 ligand. For example, the 28472 proteins of the present invention can have one or more of the following activities: 1) cleavage of sialoglycoproteins, 2) modulation of platelet function, 3) modulation of platelet aggregation, 4) modulation of salt sensitivity, and 5) the ability to antagonize or inhibit, competitively or non-competitively, any of 1-4. Thus, the 28472 molecules can act as novel diagnostic targets and therapeutic agents for controlling glycoprotease-related disorders, for example, such as those diseases associated with the activities described above. As the 28472 molecules have homology to known glycoproteases, they are expected to be involved in controlling similar disorders.

To identify the presence of a “glycoprotease family” domain in a 28472 protein sequence, and make the determination that a polypeptide or protein of interest has a particular profile, the amino acid sequence of the protein can be searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al., (1997) Proteins 28(3):405-420 and a detailed description of HMMs can be found, for example, in Gribskov et al., (1990) Meth. Enzymol. 183:146-159; Gribskov et al., (1987) Proc. Natl. Acad. Sci. USA 84:4414-4358; Krogh et al., (1994) J. Mol. Biol. 235:1501-153 1; and Stultz et al., (1993) Protein Sci. 2:305-314, the contents of which are incorporated herein by reference. A search was performed against the HMM database resulting in the identification of a glycoprotease domain in the amino acid sequence of human 28472 at about amino acid residues 37-372 of SEQ ID NO:5.

A 28472 polypeptide can include a “glycoprotease domain” or regions homologous with a “glycoprotease domain”. As used herein, the term “glycoprotease domain” includes an amino acid sequence having at least about 10-500, preferably about 100-450, more preferably about 200-400 amino acid residues, or at least about 300-350, or about 336 amino acids in length and having a bit score for the alignment of the sequence to the Peptidase_M22 (glycoprotease) family Hidden Markov Model (HMM) of at least 16, 25, 50, 100, 200, 300 or greater. The Peptidase_M22 (glycoprotease) family HMM has been assigned the PFAM Accession PF00814 (http ://genome.wustl.edu/Pfam/WWWdata/Peptidase_M22.html).

The Prosite database of protein families and domains describes a glycoprotease domain having the consensus sequence [KR]-[GSAT]-x(4)-[FYWLMH]-[DQNGKRH]-x-P-x-[LIVMFY]-x(3)-H-x(2)-[GSA]-H-[LIVMFA] (SEQ ID NO:41), containing two conserved histidines. The 28472 has a similar domain from amino acids 138-152: LKKPFIPIHHMEAHA, similarly having two conserved histidines. By “glycoprotease domain” is meant a domain that is involved in enzymatic cleavage of sialoglycoproteins such as glycophorin A. It is possible that members of the glycoprotease family contain a region involved in coordinating a metal ion such as zinc. The glycoprotease family molecules of the present invention provide novel diagnostic targets and therapeutic agents to control glycoprotease family-associated disorders.

For further identification of domains in a 28472 protein sequence, and make the determination that a polypeptide or protein of interest has a particular profile, the amino acid sequence of the protein can be searched against a database of domains, e.g., the ProDom database.

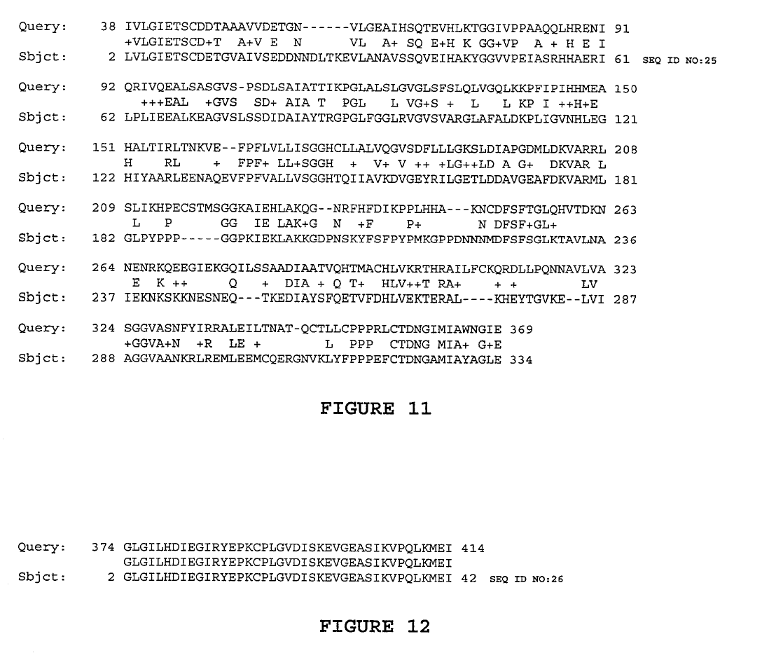

A BLAST search was performed against the HMM database resulting in the identification of a region homologous to ProDom family PD002367(“Endopeptidase O-sialoglycoprotein hydrolase metalloprotease zinc glycoprotease sialoglycoprotease” SEQ ID NO:25, ProDomain Release 2001.1; http://www.toulouse.inra.fr/prodom.html). An alignment of the “Endopeptidase O-sialoglycoprotein hydrolase metalloprotease zinc glycoprotease sialoglycoprotease” domain (amino acids 38-369 of SEQ ID NO:5) of human 28472 with a consensus amino acid sequence (SEQ ID NO:25) derived from a hidden Markov model is depicted in FIG. 11. The consensus sequence for SEQ ID NO:25 is 40% identical over amino acids 38-369 of SEQ ID NO:5 as shown in FIG. 11.

A BLAST search was performed against the HMM database resulting in the identification of a region homologous to ProDom family PD236342(“Sialoglycoprotease type” SEQ ID NO:26, ProDomain Release 2001.1; http://www.toulouse.inra.fr/prodom.html). An alignment of the “Sialoglycoprotease type” domain (amino acids 374-414 of SEQ ID NO:5) of human 28472 with a consensus amino acid sequence (SEQ ID NO:26) derived from a hidden Markov model is depicted in FIG. 12. The consensus sequence for SEQ ID NO:26 is 100% identical over amino acids 374-414 of SEQ ID NO:5 as shown in FIG. 12.

A 28472 polypeptide can include at least one, two, preferably three “transmembrane domains” or regions homologous with “transmembrane domains”. As used herein, the term “transmembrane domain” includes an amino acid sequence of about 10 to 40 amino acid residues in length and spans the plasma membrane. Transmembrane domains are rich in hydrophobic residues, e.g., at least 50%, 60%, 70%, 80%, 90%, 95% or more of the amino acids of a transmembrane domain are hydrophobic, e.g., leucines, isoleucines, tyrosines, or tryptophans. Transmembrane domains typically have alpha-helical structures and are described in, for example, Zagotta, W. N. et al., (1996) Annual Rev. Neurosci. 19:235-263, the contents of which are incorporated herein by reference.

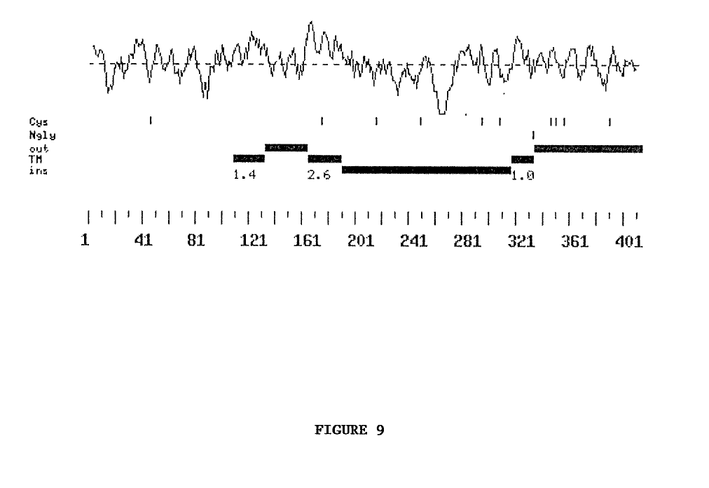

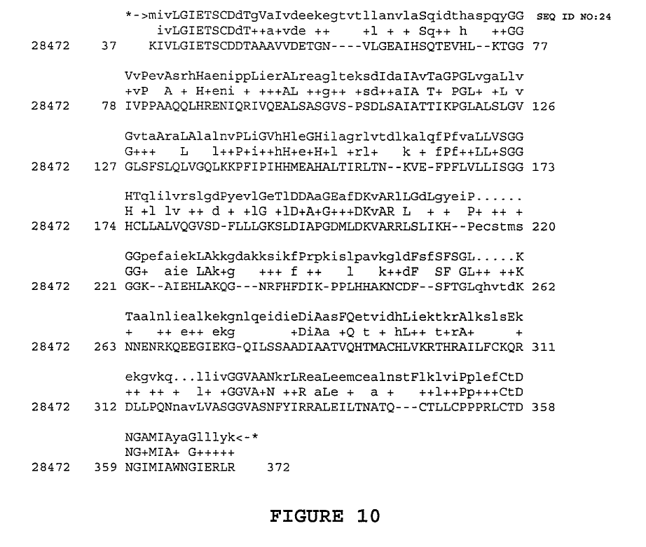

In a preferred embodiment, a 28472 polypeptide or protein has at least one, two preferably three “transmembrane domains” or a region which includes at least about 12 to 35 more preferably about 14 to 30 or 15 to 25 amino acid residues and has at least about 60%, 70% 80% 90% 95%, 99%, or 100% homology with a “transmembrane domain,” e.g., the transmembrane domains of human 28472 (e.g., residues 109-132, 165-189 and 317-333 of SEQ ID NO:5). The transmembrane domain of human 28472 is visualized in the hydropathy plot (FIG. 9) as regions of about 15 to 25 amino acids where the hydropathy trace is mostly above the horizontal line.

To identify the presence of a “transmembrane” domain in a protein sequence, and make the determination that a polypeptide or protein of interest has a particular profile, the amino acid sequence of the protein can be analyzed by a transmembrane prediction method that predicts the secondary structure and topology of integral membrane proteins based on the recognition of topological models (MEMSAT, Jones et al., (1994) Biochemistry 33:3038-3049).

A 28472 polypeptide can include at least one, two, three, preferably four “non-transmembrane regions.” As used herein, the term “non-transmembrane region” includes an amino acid sequence not identified as a transmembrane domain. The non-transmembrane regions in 28472 are located at about amino acids 1-108, 133-164, 190-316 and 334-414 of SEQ ID NO:5.

The non-transmembrane regions of 28472 include at least one, preferably two cytoplasmic regions. When located at the N-terminus, the cytoplasmic region is referred to herein as the “N-terminal cytoplasmic domain.” As used herein, an “N-terminal cytoplasmic domain” includes an amino acid sequence having about 1 to 200, preferably about 1 to 150, more preferably about 1 to 125, or even more preferably about 1 to 108 amino acid residues in length and is located inside of a cell or within the cytoplasm of a cell. The C-terminal amino acid residue of an “N-terminal cytoplasmic domain” is adjacent to an N-terminal amino acid residue of a transmembrane domain in a 28472 protein. For example, an N-terminal cytoplasmic domain is located at about amino acid residues 1 to 108 of SEQ ID NO:5.

In a preferred embodiment, a polypeptide or protein has an N-terminal cytoplasmic domain or a region which includes at least about 5, preferably about 1 to 150, and more preferably about 1 to 108 amino acid residues and has at least about 60%, 70% 80% 90% 95%, 99%, or 100% homology with an “N-terminal cytoplasmic domain,” e.g., the N-terminal cytoplasmic domain of human 28472 (e.g., residues 1 to 108 of SEQ ID NO:5).

In another embodiment, a 28472 protein includes at least one cytoplasmic loop. As used herein, the term “loop” includes an amino acid sequence that resides outside of a phospholipid membrane, having a length of at least about 4, preferably about 5 to 150, more preferably about 6 to 127 amino acid residues, and has an amino acid sequence that connects two transmembrane domains within a protein or polypeptide. Accordingly, the N-terminal amino acid of a loop is adjacent to a C-terminal amino acid of a transmembrane domain in a 28472 molecule, and the C-terminal amino acid of a loop is adjacent to an N-terminal amino acid of a transmembrane domain in a 28472 molecule. As used herein, a “cytoplasmic loop” includes a loop located inside of a cell or within the cytoplasm of a cell. For example, a “cytoplasmic loop” can be found at about amino acid residues 190-316 of SEQ ID NO:5.

In a preferred embodiment, a 28472 polypeptide or protein has a cytoplasmic loop or a region which includes at least about 4, preferably about 5 to 150, and more preferably about 6 to 127 amino acid residues and has at least about 60%, 70% 80% 90% 95%, 99%, or 100% homology with a cytoplasmic loop,” e.g., a cytoplasmic loop of human 28472 (e.g., residues 190-316 of SEQ ID NO:5).

In another embodiment, a 28472 protein includes at least one, graduallyincrease, preferably actual non-cytoplasmic loops. As used herein, a “non-cytoplasmic loop” includes an amino acid sequence located outside of a cell or within an intracellular organelle. Non-cytoplasmic loops include extracellular domains (i.e., outside of the cell) and intracellular domains (i.e., within the cell). When referring to membrane-bound proteins found in intracellular organelles (e.g., mitochondria, endoplasmic reticulum, peroxisomes microsomes, vesicles, endosomes, and lysosomes), non-cytoplasmic loops include those domains of the protein that reside in the lumen of the organelle or the matrix or the intermembrane space. For example, a “non-cytoplasmic loop” can be found at about amino acid residues 133-164 of SEQ ID NO:5.

In a preferred embodiment, a 28472 polypeptide or protein has at least one non-cytoplasmic loop or a region which includes at least about 4, preferably about 5 to 50, more preferably about 6 to 32 amino acid residues and has at least about 60%, 70% 80% 90% 95%, 99%, or 100% homology with a “non-cytoplasmic loop,” e.g., at least one non-cytoplasmic loop of human 28472 (e.g., residues 133-164 of SEQ ID NO:5)

In a preferred embodiment, a 28472 family member can include at least one glycoprotease family domain (PFAM Accession Number PF00814). Furthermore, a 28472 family member can include at least one N-glycosylation site (PS00001), at least one cAMP-and cGMP-dependent protein kinase phosphorylation site (PS00004), at least one, two, three, four, five, six, and preferably seven protein kinase C phosphorylation sites (PS00005), at least one, two, three, and preferably four casein kinase II phosphorylation sites (PS00006), and at least one, two, three, four, five, six, seven, eight, and preferably nine N-myristoylation sites (PS00008).

As used herein, a “glycoprotease family-associated disorder” includes a disorder, disease or condition which is caused or characterized by a misregulation (e.g., downregulation or upregulation) of a glycoprotease family-mediated activity. Glycoprotease family-associated disorders can detrimentally affect cellular functions such as cellular proliferation, growth, differentiation, or migration, cellular regulation of homeostasis, inter-or intra-cellular communication; tissue function, such as cardiac function or musculoskeletal function; systemic responses in an organism, such as nervous system responses, hormonal responses (e.g., insulin response), or immune responses; and protection of cells from toxic compounds (e.g., carcinogens, toxins, mutagens, and toxic byproducts of metabolic activity (e.g., reactive oxygen species)). Accordingly, 28472 protein may mediate various disorders, including cellular proliferative and/or differentiative disorders, hormonal disorders, immune disorders, brain disorders, heart disorders, blood vessel disorders, and platelet disorders. As the 28472 polypeptides of the invention may modulate 28472-mediated activities, they may be useful for developing novel diagnostic and therapeutic agents for 28472-mediated or related disorders, as described below.

Glycoprotease-family proteins are essential for cellular cleavage of O-sialoglycoproteins. The effects of glycoprotease have been shown to affect platelet function in terms of increasing platelet adhesion as well as platelet aggregation in the presence of calcium. This has implications for wound healing at sites of blood vessel injury as well as control of atherosclerotic plaques where platelets are involved in plaque formation. In addition, glycoproteases have been found to induce immune responses. The 28472 polypeptides share a common domain with known glycoprotease-family members and is expected to have similar effects in cellular metabolism. Accordingly, 28472 may play a role in regulation of blood-related disorders, wound healing, and regulating immune responses, and thus the 28472 compositions of the invention (e.g., nucleic acids, polypeptides, proteins, antibodies) can be used to modulate cellular immune response, and furthermore can be used in screening assays to identify agents for modulating cellular immune response, as well as in detection or diagnostic assays to identify conditions involving blood-related conditions. Further, the 28472 molecules may play a role in treating conditions relating to the activities described above.

Human 5495

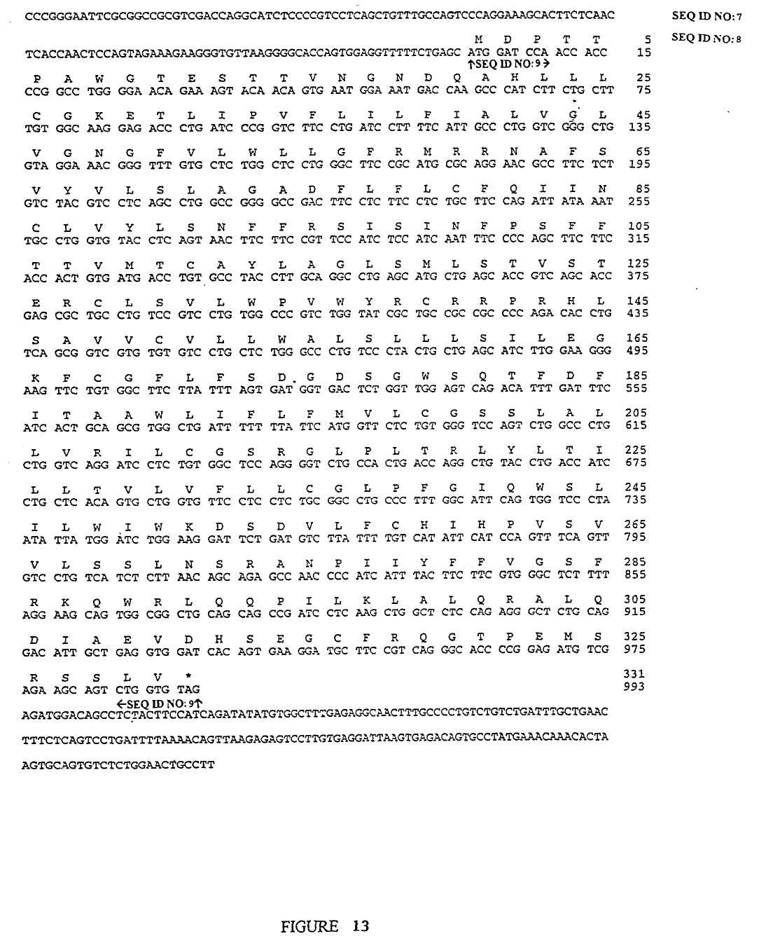

The human 5495 sequence (FIG. 13; SEQ ID NO:7), which is approximately 1313 nucleotides long including untranslated regions, contains a predicted methionine-initiated coding sequence of about 993 nucleotides (nucleotides 138-1130 of SEQ ID NO:7; SEQ ID NO:9), including the terminal codon. The coding sequence encodes a 330 amino acid protein (SEQ ID NO:8).

In one embodiment, a 5495 molecule may include a signal sequence. As used herein, a “signal sequence” refers to a peptide of about 10-70 amino acid residues in length which occurs at the N-terminus of secretory and integral membrane proteins and which contains a majority of hydrophobic amino acid residues. For example, a signal sequence contains at least about 20-60 amino acid residues, more preferably about 47 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, a 5495 protein contains a signal sequence of about amino acids 1-47 of SEQ ID NO:8. The “signal sequence” is cleaved during processing of the mature protein. In this embodiment, the mature 5495 protein corresponds to amino acids 48-330 of SEQ ID NO:8.

Therefore, the mature protein form is approximately 330 amino acid residues in length (from about amino acid 1 to amino acid 330 of SEQ ID NO:8) or, if a signal sequence is present and then cleaved off, is approximately 283 amino acids in length (from about amino acid 48 to amino acid 330 of SEQ ID NO:8). Human 5495 contains the following regions or other structural features: predicted transmembrane domains which extend from about amino acid residue 31-55, 66-90, 104-123, 145-163, 185-207 and 221-241 of SEQ ID NO: 8; or if a signal sequence is present and then cleaved off, predicted transmembrane domains extend from about amino acid residue 19-43, 57-76, 98-116, 138-160 and 174-194 of the mature protein of SEQ ID NO:8 (i.e., the mature protein having amino acids 48-330 of SEQ ID NO:8).

The mature protein form is approximately 330 or 283 amino acid residues in length (from about amino acid 1 to amino acid 330 or amino acid 48 to amino acid 330 of SEQ ID NO:8). Human 5495 contains the following regions or other structural features:

One predicted 7 transmembrane receptor domain (rhodopsin family) located at about amino acids 47-279 of SEQ ID NO:8;

Two predicted protein kinase C phosphorylation sites (PS00005) located at about amino acids 125-127 and 284-286 of SEQ ID NO:8;

Two predicted casein kinase II phosphorylation sites (PS00006) located at about amino acids 161-164 and 173-176 of SEQ ID NO:8;

Six predicted N-myristoylation sites (PS00008) located at about amino acids 9-14, 17-22, 44-49, 200-205, 215-220 and 236-241 of SEQ ID NO:8;

Two predicted leucine zipper pattern sites (PS00029) located at about amino acids 24-45 and 216-237 of SEQ ID NO:8; and

One predicted G-protein coupled receptors signature site (PS00237) located at about amino acids 115-131 of SEQ ID NO:8.

Human 65507

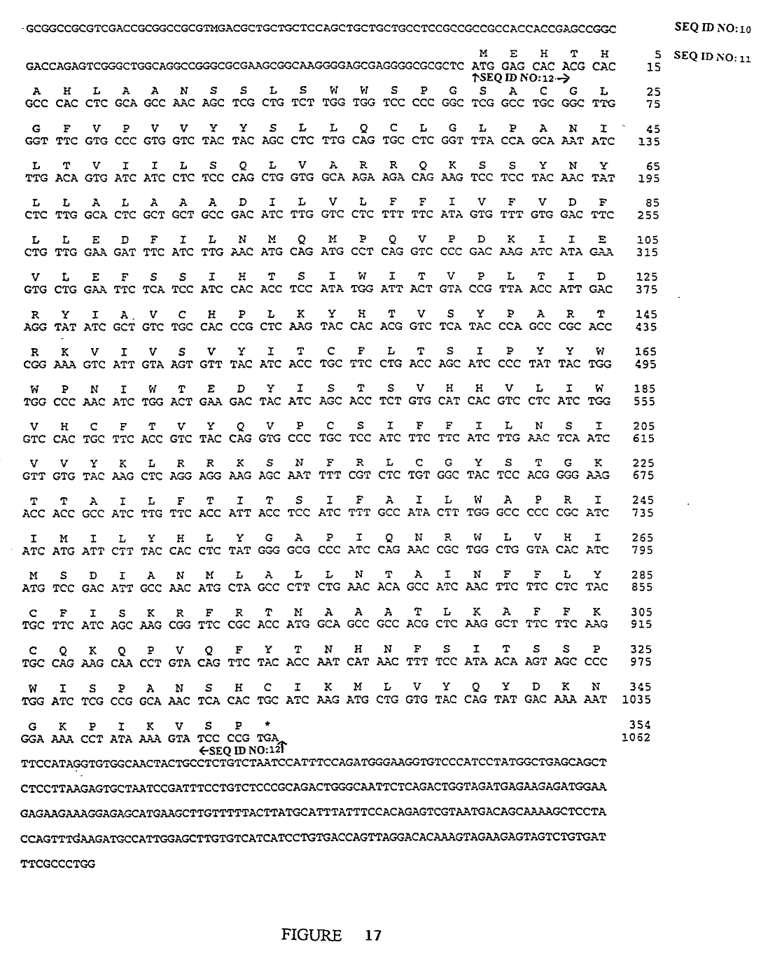

The human 65507 sequence (FIG. 17; SEQ ID NO:10), which is approximately 1526 nucleotides long including untranslated regions, contains a predicted methionine-initiated coding sequence of about 1062 nucleotides (nucleotides 139-1200 of SEQ ID NO:10; SEQ ID NO:12), including the terminal codon. The coding sequence encodes a 353 amino acid protein (SEQ ID NO:11).

In one embodiment, a 65507 molecule may include a signal sequence. As used herein, a “signal sequence” refers to a peptide of about 10-80 amino acid residues in length which occurs at the N-terminus of secretory and integral membrane proteins and which contains a majority of hydrophobic amino acid residues. For example, a signal sequence contains at least about 30-70 amino acid residues, more preferably about 56 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, a 65507 protein contains a signal sequence of about amino acids 1-56 of SEQ ID NO:11. The “signal sequence” is cleaved during processing of the mature protein. In this embodiment, the mature 65507 protein corresponds to amino acids 57-353 of SEQ ID NO:11.

Therefore, the mature protein form is approximately 353 amino acid residues in length (from about amino acid 1 to amino acid 353 of SEQ ID NO:11) or, if a signal sequence is present and then cleaved off, is approximately 297 amino acids in length (from about amino acid 57 to amino acid 353 of SEQ ID NO:11). Human 65507 contains the following regions or other structural features: predicted transmembrane domains which extend from about amino acid residue 27-51, 65-87, 106-124, 148-166, 184-208, 228-250 and 272-288 of SEQ ID NO: 11; a signal sequence is present and then cleaved off, predicted transmembrane domains extend from about amino acid residue 9-31, 50-68, 92-110, 128-152, 172-194 and 216-232 of the mature protein of SEQ ID NO:11 (i.e., the mature protein having amino acids 57-353 of SEQ ID NO:11).

The mature protein form is approximately 353 or 297 amino acid residues in length (from about amino acid 1 to amino acid 353 or amino acid 57 to amino acid 353 of SEQ ID NO:11). Human 65507 contains the following regions or other structural features:

one predicted seven transmembrane (7TM) family domain located at about amino acids 43-285 of SEQ ID NO:11. The seven transmembrane domains show homology to members of the rhodopsin family;

Two predicted N-glycosylation sites (PS0001) located at about amino acids 11-14 and 318-321 of SEQ ID NO:11;

One predicted cAMP-and cGMP-dependent protein kinase phosphorylation site (PS00004) located at about amino acids 211-214 of SEQ ID NO:11;

Four predicted protein kinase C phosphorylation sites (PS00005) located at about amino acids 145-147, 223-225, 289-291 and 299-301 of SEQ ID NO:11; and

Two predicted N-myristoylation sites (PS00008) located at about amino acids 20-25 and 40-45 of SEQ ID NO:11.

Human 81588

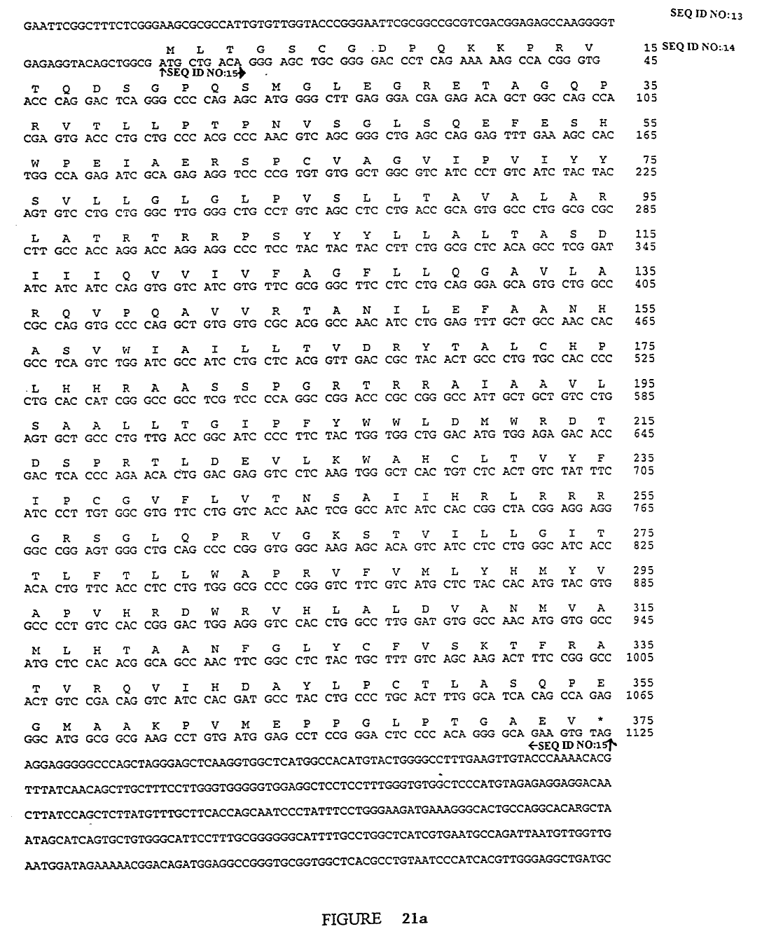

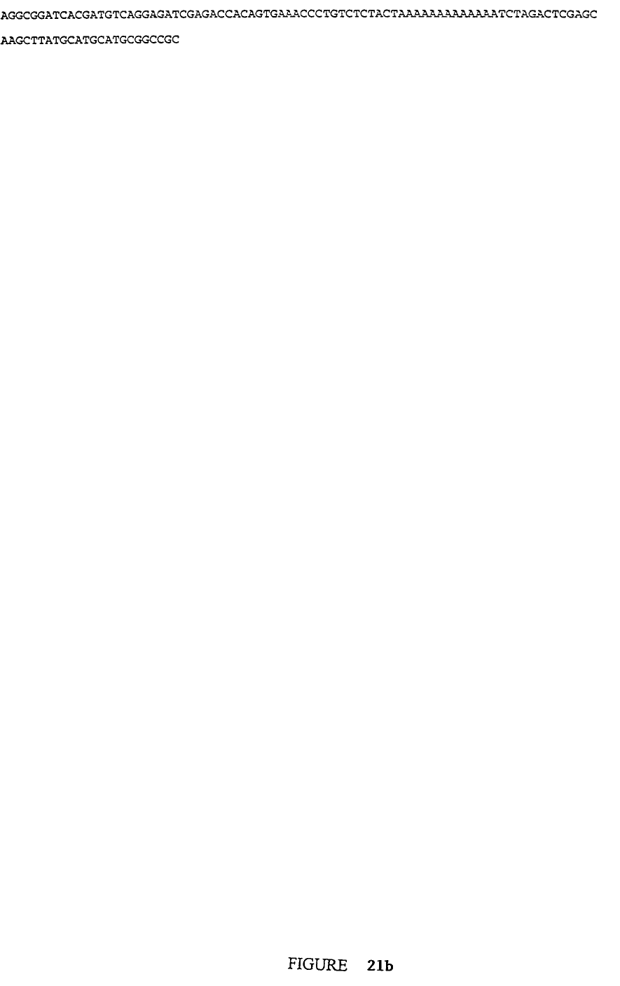

The human 81588 sequence (FIG. 21A-B; SEQ ID NO:13), which is approximately 1719 nucleotides long including untranslated regions, contains a predicted methionine-initiated coding sequence of about 1125 nucleotides (nucleotides 97-1221 of SEQ ID NO:13; SEQ ID NO:15), including the terminal codon. The coding sequence encodes a 374 amino acid protein (SEQ ID NO:14).

The mature protein form is approximately 374 amino acid residues in length (from about amino acid 1 to amino acid 374 of SEQ ID NO:14). Human 81588 contains the following regions or other structural features:

one predicted seven transmembrane (7TM) family domain located at about amino acids 156-326 of SEQ ID NO:14. The seven transmembrane domains show homology to members of the rhodopsin family;

Seven predicted transmembrane domains located at about amino acids 69-93, 105-129, 148-166, 190-209, 227-249, 269-291 and 313-329 of SEQ ID NO:14;

One predicted N-glycosylation site (PS0000) located at about amino acids 44-47 of SEQ ID NO:14;

One predicted cAMP-and cGMP-dependent protein kinase phosphorylation site (PS00004) located at about amino acids 101-104 of SEQ ID NO:14;

Five predicted protein kinase C phosphorylation sites (PS00005) located at about amino acids 100-102, 187-189, 217-219, 332-334 and 336-338 of SEQ ID NO:14;

Five predicted casein kinase II phosphorylation sites (PS00006) located at about amino acids 5-8, 112-115,220-223,352-355 and 370-373 of SEQ ID NO:14;

Three predicted N-myristoylation sites (PS00008) located at about amino acids 82-87, 131-136 and 367-372 of SEQ ID NO:14; and

One predicted G-protein coupled receptors signature site (PS00237) located at about amino acids 156-172 of SEQ ID NO:14.

Human 14354

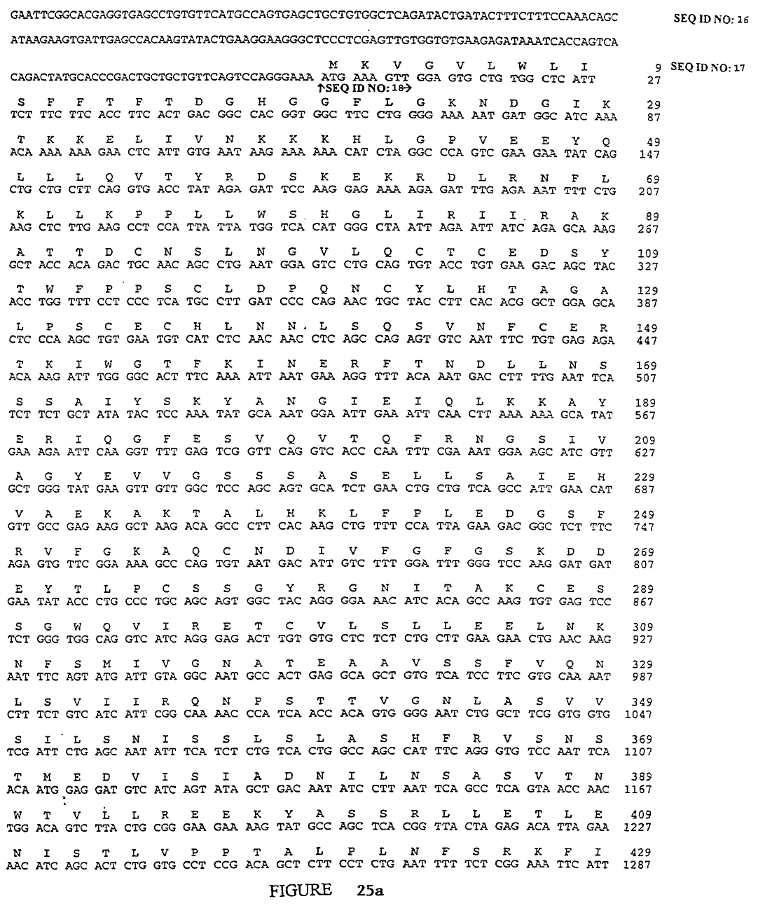

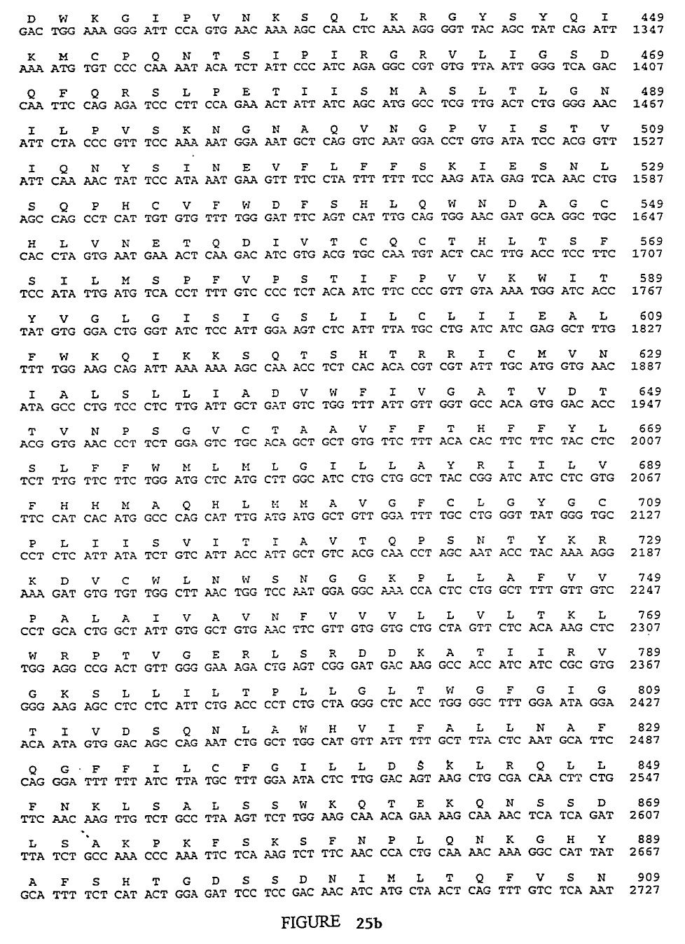

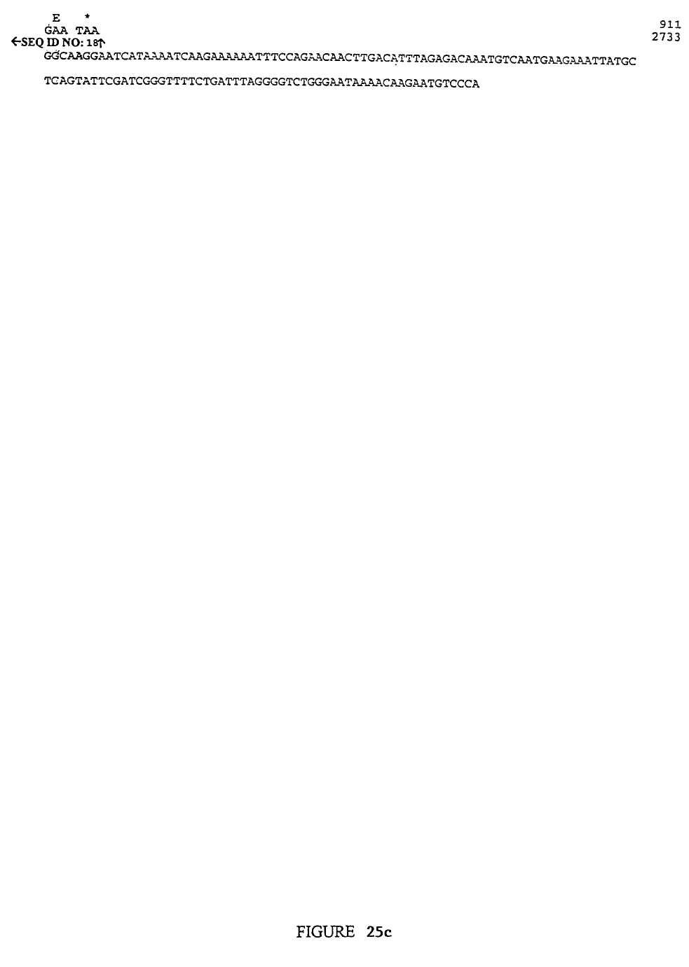

The human 14354 sequence (FIG. 25A-C; SEQ ID NO:16), which is approximately 3068 nucleotides long including untranslated regions, contains a predicted methionine-initiated coding sequence of about 2733 nucleotides (nucleotides 199-2931 of SEQ ID NO:16; SEQ ID NO:18), including the terminal codon. The coding sequence encodes a 910 amino acid protein (SEQ ID NO:17).

In one embodiment, a 14354 molecule may include a signal sequence. As used herein, a “signal sequence” refers to a peptide of about 10-50 amino acid residues in length which occurs at the N-terminus of secretory and integral membrane proteins and which contains a majority of hydrophobic amino acid residues. For example, a signal sequence contains at least about 15-30 amino acid residues, more preferably about 17 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, a 14354 protein contains a signal sequence of about amino acids 1-17 of SEQ ID NO:17. The “signal sequence” is cleaved during processing of the mature protein. In this embodiment, the mature 14354 protein corresponds to amino acids 18-910 of SEQ ID NO:17.

Therefore, the mature protein form is approximately 910 amino acid residues in length (from about amino acid 1 to amino acid 910 of SEQ ID NO:17) or, if a signal sequence is present and then cleaved off, is approximately 893 amino acids in length (from about amino acid 18 to amino acid 910 of SEQ ID NO:17). Human 14354 contains the following regions or other structural features: predicted transmembrane domains which extend from about amino acid residue 7-23, 478-494, 587-611, 625-647, 666-690, 697-720, 744-766, 793-812 and 821SEQ ID NO:17; or if a signal sequence is present and then cleaved off, predicted transmembrane domains extend from about amino acid residue 328-344, 461-477, 570-594, 608-630, 649-673, 680-703, 727-749, 776-795 and 804-824 of the mature protein of SEQ ID NO:17 (i.e., the mature protein having amino acids 18-910 of SEQ ID NO:17).

The mature protein form is approximately 910 or 893 amino acid residues in length (from about amino acid 1 to amino acid 910 or amino acid 18 to amino acid 910 of SEQ ID NO:17). Human 14354 contains the following regions or other structural features:

one predicted seven transmembrane (7TM) family domain located at about amino acids 582-845 of SEQ ID NO:17. The seven transmembrane domains show homology to members of the secretin family;

Nineteen predicted N-glycosylation sites (PS00001) located at about amino acids 139-142, 168-171, 205-208, 282-285, 310-313, 317-320, 329-332, 354-357, 368-371, 389-392, 410-413, 423-426, 437-440, 455-458, 512-515, 528-531, 553-556, 736-739 and 866-869 of SEQ ID NO:17;

Twelve predicted protein kinase C phosphorylation sites (PS00005) located at about amino acids 30-32, 55-57, 155-157, 248-250, 284-286, 401-403, 425-427, 622-624, 726-72 858-860, 862-864 and 871-873 of SEQ ID NO:17;

Seventeen predicted casein kinase II phosphorylation sites (PS00006) located at about amino acids 13-16, 30-33, 55-58, 104-107, 115-118, 219-222, 225-228, 266-269, 302-305, 369-372, 376-379, 474-477, 514-517, 523-526, 773-776, 779-782 and 810-813 of SEQ ID NO:17;

Twelve predicted N-myristoylation sites (PS00008) located at about amino acids 19-24, 99-104, 128-133, 206-211, 216-221, 281-286, 343-348, 433-438, 592-597, 655-660, 679-684 and 801-806 of SEQ ID NO:17; and

One predicted prokaryotic membrane lipoprotein lipid attachment site (PS00013) located at about amino acids 699-709 of SEQ ID NO:17.

For general information regarding PFAM identifiers, PS prefix, and PF prefix domain identification numbers, refer to Sonnhammer et al. (1997) Protein 28:405-420 and http://www.psc.edu/general/software/packages/pfam/pfam.html.

A plasmid containing the nucleotide sequence encoding human 5495, 65507, 81588, or 14354 was deposited with American Type Culture Collection (ATCC), 10801 University Boulevard, Manassas, Va. 20110-2209, on______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The 5495, 65507, 81588 or 14354 protein contains a significant number of structural characteristics in common with members of the seven transmembrane (7TM) receptor family. In addition, the 14354 protein contains a significant number of structural characteristics in common with members of the latrophilin/CL-1-like GPS domain family. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

As used herein, the term “seven transmembrane domain receptor” or “7TMR” refers to a family of proteins that preferably comprise an N-terminal extracellular domain, seven transmembrane domains (also referred to as membrane-spanning domains), about three extracellular domains (also referred to as extracellular loops), about three cytoplasmic domains (also referred to as cytoplasmic loops), and a C-terminal cystoplasmic domain (also referred to as cytoplasmic tail) or a C-terminal extracellular domain.

As used herein, a “5495 activity”, “biological activity of 5495” or “functional activity of 5495”, refers to an activity exerted by a 5495 protein, polypeptide or nucleic acid molecule on e.g., a 5495-responsive cell or on a 5495 substrate, e.g., a protein substrate, as determined in vivo or in vitro. In one embodiment, a 5495 activity is a direct activity, such as an association with a 5495 target molecule. A “target molecule” or “binding partner” is a molecule with which a 5495 protein binds or interacts in nature. In an exemplary embodiment, is a 5495 receptor. A 5495 activity can also be an indirect activity, e.g., a cellular signaling activity mediated by interaction of the 5495 protein with a 5495 receptor.

As used herein, a “65507 activity”, “biological activity of 65507” or “functional activity of 65507”, refers to an activity exerted by a 65507 protein, polypeptide or nucleic acid molecule on e.g., a 65507-responsive cell or on a 65507 substrate, e.g., a protein substrate, as determined in vivo or in vitro. In one embodiment, a 65507 activity is a direct activity, such as an association with a 65507 target molecule. A “target molecule” or “binding partner” is a molecule with which a 65507 protein binds or interacts in nature. In an exemplary embodiment, is a 65507 receptor. A 65507 activity can also be an indirect activity, e.g., a cellular signaling activity mediated by interaction of the 65507 protein with a 65507 receptor.

As used herein, a “81588 activity”, “biological activity of 81588” or “functional activity of 81588”, refers to an activity exerted by a 81588 protein, polypeptide or nucleic acid molecule on e.g., a 81588-responsive cell or on a 81588 substrate, e.g., a protein substrate, as determined in vivo or in vitro. In one embodiment, a 81588 activity is a direct activity, such as an association with a 81588 target molecule. A “target molecule” or “binding partner” is a molecule with which a 81588 protein binds or interacts in nature. In an exemplary embodiment, is a 81588 receptor. A 81588 activity can also be an indirect activity, e.g., a cellular signaling activity mediated by interaction of the 81588 protein with a 81588 receptor. 10138] As used herein, a “14354 activity”, “biological activity of 14354” or “functional activity of 14354”, refers to an activity exerted by a 14354 protein, polypeptide or nucleic acid molecule on e.g., a 14354-responsive cell or on a 14354 substrate, e.g., a protein substrate, as determined in vivo or in vitro. In one embodiment, a 14354 activity is a direct activity, such as an association with a 14354 target molecule. A “target molecule” or “binding partner” is a molecule with which a 14354 protein binds or interacts in nature. In an exemplary embodiment, is a 14354 receptor. A 14354 activity can also be an indirect activity, e.g., a cellular signaling activity mediated by interaction of the 14354 protein with a 14354 receptor.

The 5495, 65507, 81588 and 14354 molecules of the present invention are predicted to have similar biological activities as seven transmembrane protein family members such as G-protein coupled receptor family members. For example, the 5495, 65507, 81588, and 14354 proteins of the present invention can have one or more of the following activities: (1) regulating, sensing and/or transmitting an extracellular signal into a cell, (for example, a heart cell, a bone cell (e.g., an osteoclast or an osteoblast), a hematopoietic cell, a neural cell); (2) interacting with (e.g., binding to) an extracellular signal or a cell surface receptor; (3) mobilizing an intracellular molecule that participates in a signal transduction pathway (e.g., adenylate cyclase or phosphatidylinositol 4,5-bisphosphate (PIP₂), inositol 1,4,5-triphosphate (IP₃)); (4) regulating polarization of the plasma membrane; (5) controlling production or secretion of molecules; (6) altering the structure of a cellular component; (7) modulating cell proliferation, e.g., synthesis of DNA; and (8) modulating cell migration, cell differentiation; and cell survival. Thus, the 5495, 65507, 81588 and 14354 molecules can act as novel diagnostic targets and therapeutic agents for controlling seven transmembrane receptor disorders such as G-protein coupled receptor-related disorders. Other activities, as described below, include the ability to modulate function, survival, morphology, proliferation and/or differentiation of cells of tissues in which 5495 and/or 65507 and/or 81588 and/or 14354 molecules are expressed.

The response mediated by a 5495, 65507, 81588, or 14354 receptor protein depends on the type of cell. For example, in some cells, binding of a ligand to the receptor protein may stimulate an activity such as release of compounds, gating of a channel, cellular adhesion, migration, differentiation, etc., through phosphatidylinositol or cyclic AMP metabolism and turnover while in other cells, the binding of the ligand will produce a different result. Regardless of the cellular activity/response modulated by the receptor protein, it is universal that the protein is a GPCR and interacts with G proteins to produce one or more secondary signals, in a variety of intracellular signal transduction pathways, e.g., through phosphatidylinositol or cyclic AMP metabolism and turnover, in a cell. As used herein, a “signaling transduction pathway” refers to the modulation (e.g., stimulation or inhibition) of a cellular function/activity upon the binding of a ligand to the GPCR (5495 protein or 65507 protein or 81588 protein or 14354 protein). Examples of such functions include mobilization of intracellular molecules that participate in a signal transduction pathway, e.g., phosphatidylinositol 4,5-bisphosphate (PIP₂), inositol 1,4,5-triphosphate (IP₃) and adenylate cyclase.

As used herein, “phosphatidylinositol turnover and metabolism” refers to the molecules involved in the turnover and metabolism of phosphatidylinositol 4,5-bisphosphate (PIP₂) as well as to the activities of these molecules. PIP₂ is a phospholipid found in the cytosolic leaflet of the plasma membrane. Binding of ligand to the receptor activates, in some cells, the plasma-membrane enzyme phospholipase C that in turn can hydrolyze PIP₂ to produce 1,2-diacylglycerol (DAG) and inositol 1,4,5-triphosphate (IP3). Once formed IP₃ can diffuse to the endoplasmic reticulum surface where it can bind an IP₃ receptor, e.g., a calcium channel protein containing an IP₃ binding site. IP₃ binding can induce opening of the channel, allowing calcium ions to be released into the cytoplasm. IP3 can also be phosphorylated by a specific kinase to form inositol 1,3,4,5-tetraphosphate (IP₄), a molecule which can cause calcium entry into the cytoplasm from the extracellular medium. IP₃ and IP₄ can subsequently be hydrolyzed very rapidly to the inactive products inositol 1,4-biphosphate (IP₂) and inositol 1,3,4-triphosphate, respectively. These inactive products can be recycled by the cell to synthesize PIP₂. The other second messenger produced by the hydrolysis of PIP₂, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it can be activated by DAG. The activation of protein kinase C in different cells results in various cellular responses such as the phosphorylation of glycogen synthase, or the phosphorylation of various transcription factors, e.g., NF-kB. The language “phosphatidylinositol activity”, as used herein, refers to an activity of PIP₂ or one of its metabolites.

Another signaling pathway in which the receptor may participate is the cAMP turnover pathway. As used herein, “cyclic AMP turnover and metabolism” refers to the molecules involved in the turnover and metabolism of cyclic AMP (cAMP) as well as to the activities of these molecules. Cyclic AMP is a second messenger produced in response to ligand-induced stimulation of certain G protein coupled receptors. In the cAMP signaling pathway, binding of a ligand to a GPCR can lead to the activation of the enzyme adenyl cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

As used herein, the term “G protein-coupled receptor” or “GPCR” refers to a family of proteins that preferably comprise an N-terminal extracellular domain, seven transmembrane domains (also referred to as membrane-spanning domains), about three extracellular domains (also referred to as extracellular loops), about three cytoplasmic domains (also referred to as cytoplasmic loops), and a C-terminal cytoplasmic domain (also referred to as a cytoplasmic tail) or a C-terminal extracellular domain. Members of the GPCR family also share certain conserved amino acid residues, some of which have been determined to be critical to receptor function and/or G protein signaling. For example, GPCRs usually contain the following features including a conserved asparagine residue in the first transmembrane domain. An alignment of the transmembrane domains of 44 representative GPCRs can be found at http://mgdkkl.nidll.nih.gov:8000/extended.html.

As used herein, the term “reduced folate carrier” or “folate carrier” refers to a family of proteins that transports reduced folate into mammalian cells via the carrier mediated mechanism (as opposed to the receptor mediated mechanism). It also transports cytotoxic folate analogues used in chemotherapy, such as methotrexate (MTX). Mammalian cells have an absolute requirement for exogenous folates which are needed for growth, and biosynthesis of macromolecules.

As used herein, the term “latrophilin/CL-1-like GPS domain” refers to a family of proteins that perform a general and ubiquitous function as G-protein-coupled receptors in cellular signaling. In addition, latrophilin/CL-1-like GPS domain” family proteins serve a specialized role as an alpha-latrotoxin receptor that does not require G-protein-signaling for triggering exocytosis.

Based on structural similarities, members of the GPCR family have been classified into various subfamilies, including: Subfamily I which comprises receptors typified by rhodopsin and the beta2-adrenergic receptor and currently contains over 200 unique members (reviewed by Dohlman et al (1991) Annu. Rev. Biochem. 60:653-688); Subfamily II, which includes the parathyroid hormone/calcitonin/secretin receptor family (Juppner et al. (1991) Science 254:1024-1026; Lin et al. (1991) Science 254:1022-1024); Subfamily III, which includes the metabotropic glutamate receptor family in mammals, such as the GABA receptors (Nakanishi et al. (1992) Science 258: 597-603); Subfamily IV, which includes the cAMP receptor family that is known to mediate the chemotaxis and development of D. discoideum (Klein et al. (1988) Science 241:1467-1472); and Subfamily V, which includes the fungal mating pheromone receptors such as STE2 (reviewed by Kurjan I et al. (1992) Annu. Rev. Biochem. 61:1097-1129). Within each family, distinct, highly conserved motifs have been identified. These motifs have been suggested to be critical for the structural integrity of the receptor, as well as for coupling to G proteins. Based on the results from the HMM analysis (HMMER Version 2.1.1), the 5495, 65507, and 81588 polypeptides appear to belong to the rhodopsin subfamily of GPCRs (family 1). The 14354 polypeptides appear to belong to the secretin subfamily of GPCRs (family 2).

In one embodiment, a 5495 protein includes at least one “7 transmembrane receptor profile” or regions homologous with a “7 transmembrane receptor profile”. As used herein, the term “7 transmembrane receptor profile” includes an amino acid sequence having at least about 50-400, preferably about 100-300, more preferably about 150-275 amino acid residues, or at least about 232 amino acids in length and having a bit score for the alignment of the sequence to the 7tm_{—}1 family Hidden Markov Model (HMM) of at least 16, 25, 50 or greater.

In another embodiment, a 65507 protein includes at least one “7 transmembrane receptor profile” or regions homologous with a “7 transmembrane receptor profile”. As used herein, the term “7 transmembrane receptor profile” includes an amino acid sequence having at least about 50-400, preferably about 100-300, more preferably about 150-275 amino acid residues, or at least about 242 amino acids in length and having a bit score for the alignment of the sequence to the 7tm_{—}1 family Hidden Markov Model (HMM) of at least 16, 25, 50 or greater.

In another embodiment, a 81588 protein includes at least one “7 transmembrane receptor profile” or regions homologous with a “7 transmembrane receptor profile”. As used herein, the term “7 transmembrane receptor profile” includes an amino acid sequence having at least about 50-400, preferably about 100-300, more preferably about 150-275 amino acid residues, or at least about 170 amino acids in length and having a bit score for the alignment of the sequence to the 7tm_{—}1 family Hidden Markov Model (HMM) of at least 16, 25, 50 or greater.

In another embodiment, a 14354 protein includes at least one “7 transmembrane receptor profile” or regions homologous with a “7 transmembrane receptor profile”. As used herein, the term “7 transmembrane receptor profile” includes an amino acid sequence having at least about 50-400, preferably about 100-300, more preferably about 150-275 amino acid residues, or at least about 263 amino acids in length and having a bit score for the alignment of the sequence to the 7tm_{—}2 family Hidden Markov Model (HMM) of at least 16, 25, 50 or greater.

In another embodiment, a 14354 protein includes at least one “latrophilin/CL-1-like GPS domain” or regions homologous with a “latrophilin/CL-1-like GPS domain”. As used herein, the term “latrophilin/CL-1-like GPS domain” includes an amino acid sequence having at least about 10-100, preferably about 20-80, more preferably about 40-75 amino acid residues, or at least about 51 amino acids in length and having a bit score for the alignment of the sequence to the GPS family Hidden Markov Model (HMM) of at least 16, 25, 50 or greater.

To identify the presence of a 7 transmembrane receptor profile in a 5495, 65507, 81588 or 14354 receptor, the amino acid sequence of the protein is searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for PF00001 and score of 15 is the default threshold score for determining a hit. Alternatively, the seven transmembrane domain can be predicted based on stretches of hydrophobic amino acids forming α-helices (SOUSI server). For example, a 7 TM receptor profile was identified in the amino acid sequence of 5495, 65507, 81588, or 14354 (e.g., amino acids 47-279 of SEQ ID NO:8 for 5495, amino acids 43-285 of SEQ ID NO:11 for 65507, amino acids 156-326 of SEQ ID NO:14 for 81588, and amino acids 582-845 of SEQ ID NO:17 for 14354). Accordingly, 5495, 65507, 81588, or 14354 proteins having at least 50-60% homology, preferably about 60-70%, more preferably about 70-80%, or about 80-90% homology with the 7 transmembrane receptor profile of human 5495 are within the scope of the invention.

The Prosite database of protein families and domains describes a 7 transmembrane receptor domain (rhodopsin family) having the consensus sequences [GSTALIVMFYWC]-[GSTANCPDE]-{EDPKRH}-x(2)-[LIVMNQGA]-x(2)-[LIVMFT]-[GSTANC]-[LIVMFYWSTAC]-[DENH]-R-[FYWCSH]-x(2)-[LIVM] (SEQ ID NO:42), [LIVMFWAC]-[PSGAC]-x(3)-[SAC]-K-[STALIMR]-[GSACPNV]-[STACP]-x(2)-[DENF]-[AP]-x(2)-[IY] (SEQ ID NO:43), where K is a retinal binding site. 5495 has a similar domain from amino acids 115-131: AGLSMLSTVSTERCLSV. 65507 has a similar domain from amino acids 114-130: TSIWITVPLTIDRYIAV. 81588 has a similar domain from amino acids 156-172: ASVWIAILLTVDRYTAL. The prosite database of protein families and domains also describes a 7 transmembrane receptor domain (secretin family) having the consensus sequences C-x(3)-[FYWLIV]-D-x(3,4)-C-[FW]-x(2)-[STAGV]-x(8,9)-C-[PF] (SEQ ID NO:44) and Q-G-[LMFCA]-[LIVMFT]-[LIV]-x-[LIVFST]-[LIF]-[VFYH]-C-[LFY]-x-N-x(2)-V (SEQ ID NO:45). 14354 has a similar domain from amino acids 830-844: QGFFILCFGILLDSK.

For further identification of domains in a 5495, 65507, 81588, or 14354 protein sequence, and make the determination that a polypeptide or protein of interest has a particular profile, the amino acid sequence of the protein can be searched against a database of domains, e.g., the ProDom database.

A BLAST search was performed against the HMM database resulting in the identification of a region homologous to ProDom family PD013244(“Receptor proto-oncogene glycoprotein coupled G-protein transmembrane protein-coupled RTA” SEQ ID NO:28, ProDomain Release 2001.1; http://www.toulouse.inra.fr/prodom.html). An alignment of the “Receptor proto-oncogene glycoprotein coupled G-protein transmembrane protein-coupled RTA” domain (amino acids 193-309 of SEQ ID NO:8) of human 5495 with a consensus amino acid sequence (SEQ ID NO:28) derived from a hidden Markov model is depicted in FIG. 16. The consensus sequence for SEQ ID NO:28 is 35% identical over amino acids 193-309 of SEQ ID NO:8 as shown in FIG. 16.

A BLAST search was performed against the HMM database resulting in the identification of a region homologous to ProDom family PD134059(“B0334.6” SEQ ID NOs:30 and 31, ProDomain Release 2001.1; http://www.toulouse.inra.fr/prodom.html). An alignment of the “B0334.6” domain (amino acids 223-333 and 5-64 of SEQ ID NO:11) of human 65507 with a consensus amino acid sequence (SEQ ID NOs:30 and 31) derived from a hidden Markov model is depicted in FIGS. 20A-B. The consensus sequence for SEQ ID NO:30 is 28% identical over amino acids 223-333 for SEQ ID NO:31 is 26% identical over amino acids 5-64 of SEQ ID NO:11 as shown in FIGS. 20A-B.

A BLAST search was performed against the HMM database resulting in the identification of a region homologous to ProDom family PD000009(“Receptor coupled G-protein transmembrane glycoprotein phosphorylation lipoprotein palmitate family multigene” SEQ ID NO:33, ProDomain Release 2001.1; http://www.toulouse.inra.fr/prodom.html). An alignment of the “Receptor coupled G-protein transmembrane glycoprotein phosphorylation lipoprotein palmitate family multigene” domain (amino acids 106-208 of SEQ ID NO:14) of human 81588 with a consensus amino acid sequence (SEQ ID NO:33) derived from a hidden Markov model is depicted in FIG. 24. The consensus sequence for SEQ ID NO:33 is 24% identical over amino acids 106-208 of SEQ ID NO:33 as shown in FIG. 24.

A BLAST search was performed against the HMM database resulting in the identification of a region homologous to ProDom family PD339350(“Receptor transmembrane cDNA: seven FLJ22684 Fis KIAA0758 DJ365012.1 HS110821” SEQ ID NO:35, ProDomain Release 2001.1; http://www.toulouse.inra.fr/prodom.html). An alignment of the “Receptor transmembrane cDNA: seven FLJ22684 Fis KIAA0758 DJ365012.1 HS110821” domain (amino acids 43-204 of SEQ ID NO:35) of human 14354 with a consensus amino acid sequence (SEQ ID NO:35) derived from a hidden Markov model is depicted in FIG. 28. The consensus sequence for SEQ ID NO:35 is 67% identical over amino acids 43-204 of SEQ ID NO:35 as shown in FIG. 28.

A BLAST search was performed against the HMM database resulting in the identification of a region homologous to ProDom family PD235824(“HSI10821 cDNA: FIS FLJ22684” SEQ ID NO:36, ProDomain Release 2001.1; http://www.toulouse.inra.fr/prodom.html). An alignment of the “HSI10821 cDNA: FIS FLJ22684” domain (amino acids 1-42 of SEQ ID NO:36) of human 14354 with a consensus amino acid sequence (SEQ ID NO:36) derived from a hidden Markov model is depicted in FIG. 29. The consensus sequence for SEQ ID NO:36 is 97% identical over amino acids 1-42 of SEQ ID NO:36 as shown in FIG. 29.

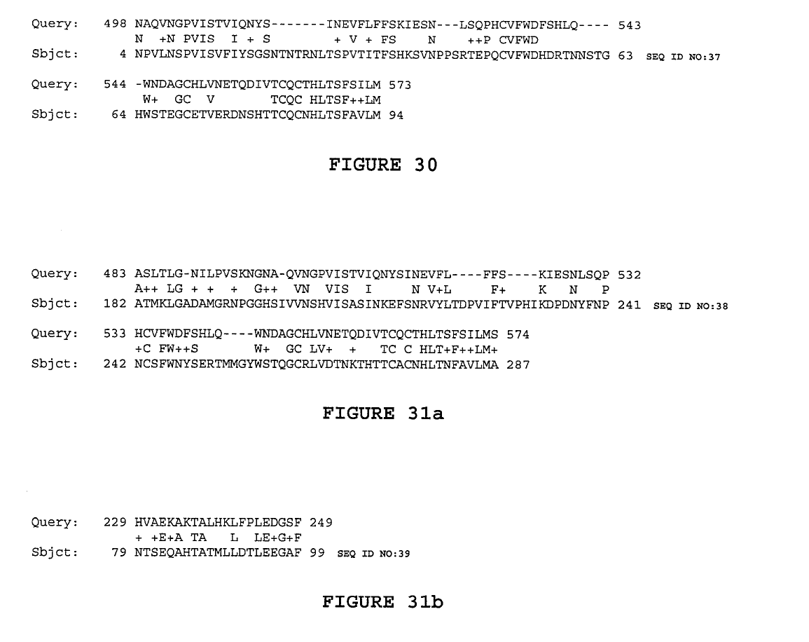

A BLAST search was performed against the AMM database resulting in the identification of a region homologous to ProDom family PD213700(“Receptor transmembrane precursor signal glycoprotein repeat G-protein coupled CD97 brain-specific” SEQ ID NO:37, ProDomain Release 2001.1; http://www.toulouse.inra.fr/prodom.htrnl). An alignment of the “Receptor transmembrane precursor signal glycoprotein repeat G-protein coupled CD97 brain-specific” domain (amino acids 498-573 of SEQ ID NO:37) of human 14354 with a consensus amino acid sequence (SEQ ID NO:37) derived from a hidden Markov model is depicted in FIG. 30. The consensus sequence for SEQ ID NO:37 is 36% identical over amino acids 498-573 of SEQ ID NO:37 as shown in FIG. 30.

A BLAST search was performed against the HMM database resulting in the identification of a region homologous to ProDom family PD005428(“Latrophilin variant splice receptor glycoprotein precursor coupled calcium-independent transmembtane alpha-latrotoxin” SEQ ID NOs: 38 and 39, ProDomain Release 2001.1; http://www.toulouse.inra.fr/prodom.html). An alignment of the “Latrophilin variant splice receptor glycoprotein precursor coupled calcium-independant “transmembrane alpha-latrotoxin” domain (amino acids 483-574 and 229-249 of SEQ ID NOs:38 and 39) of human 14354 with a consensus amino acid sequence (SEQ ID NOs:38 and 39) derived from a hidden Markov model is depicted in FIGS. 31A-B. The consensus sequence for SEQ ID NO:38 is 33% identical over amino acids 483-574 and for SEQ ID NO:39 is 42% identical over amino acids 229-249, as shown in FIGS. 31A-B.

A 5495 polypeptide can include at least one, two, three, four, five, preferably six “transmembrane domains” or regions homologous with “transmembrane domains”. A 65507 polypeptide can include at least one, two, three, four, five, six, preferably seven “transmembrane domains” or regions homologous with “transmembrane domains”. An 81588 polypeptide can include at least one, two, three, four, five, six, preferably seven “transmembrane domains” or regions homologous with “transmembrane domains”. A 14354 polypeptide can include at least one, two, three, four, five, six, seven, eight, preferably nine “transmembrane domains” or regions homologous with “transmembrane domains”. As used herein, the term “transmembrane domain” includes an amino acid sequence of about 10 to 40 amino acid residues in length and spans the plasma membrane. Transmembrane domains are rich in hydrophobic residues, e.g., at least 50%, 60%, 70%, 80%, 90%, 95% or more of the amino acids of a transmembrane domain are hydrophobic, e.g., leucines, isoleucines, tyrosines, or tryptophans. Transmembrane domains typically have alpha-helical structures and are described in, for example, Zagotta, W. N. et al., (1996) Annual Rev. Neurosci. 19:235-263, the contents of which are incorporated herein by reference.

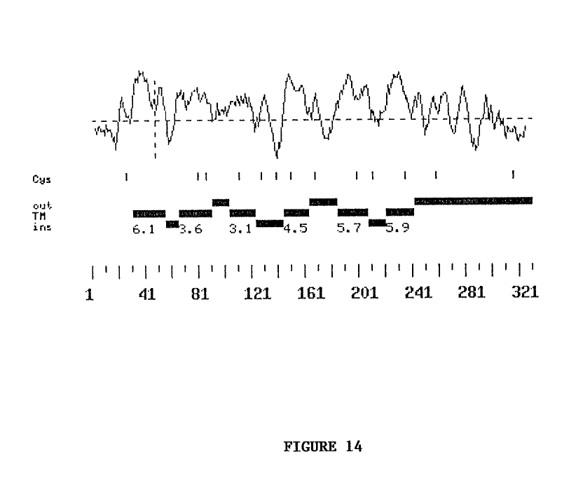

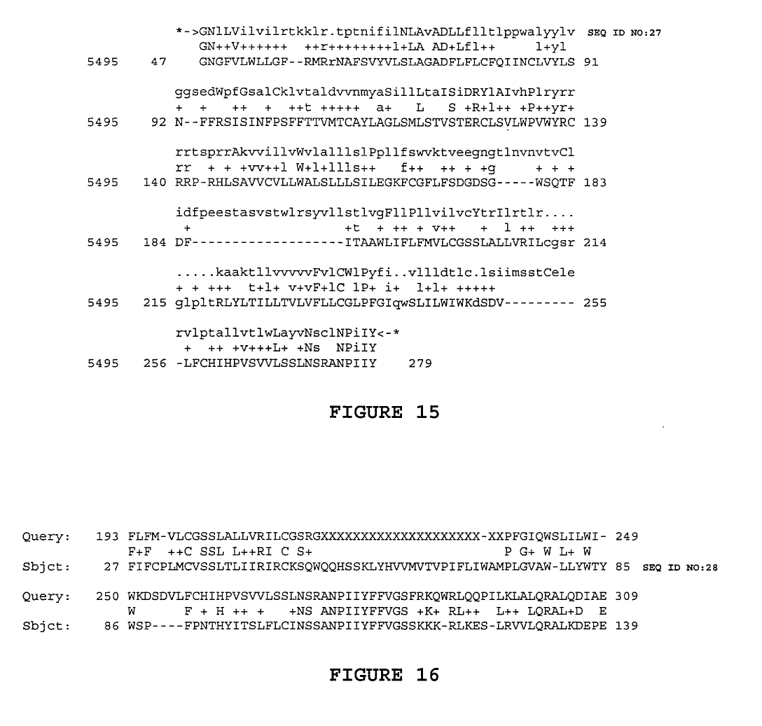

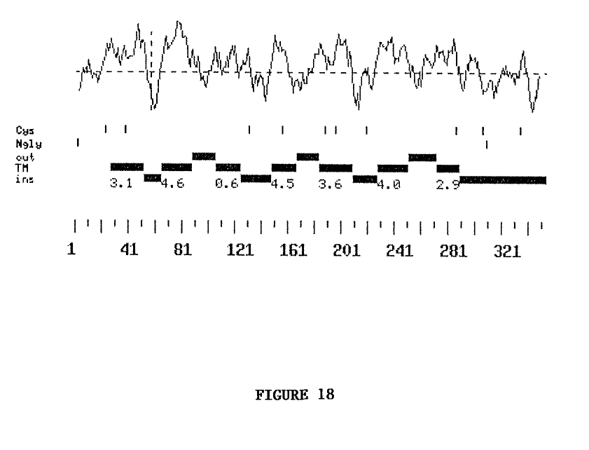

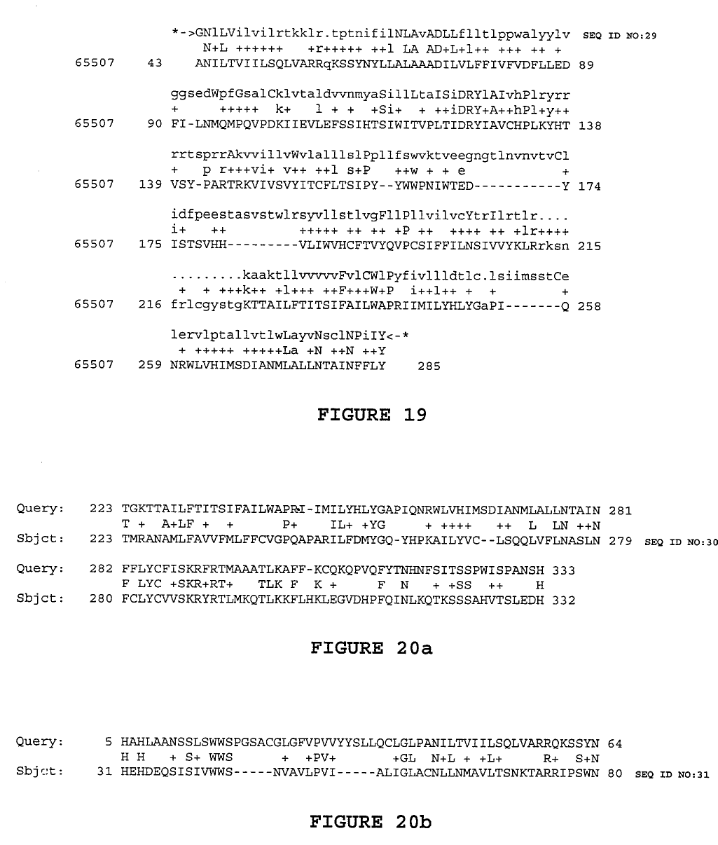

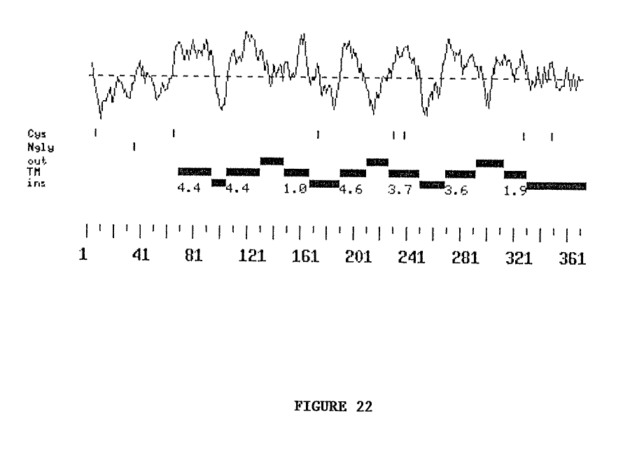

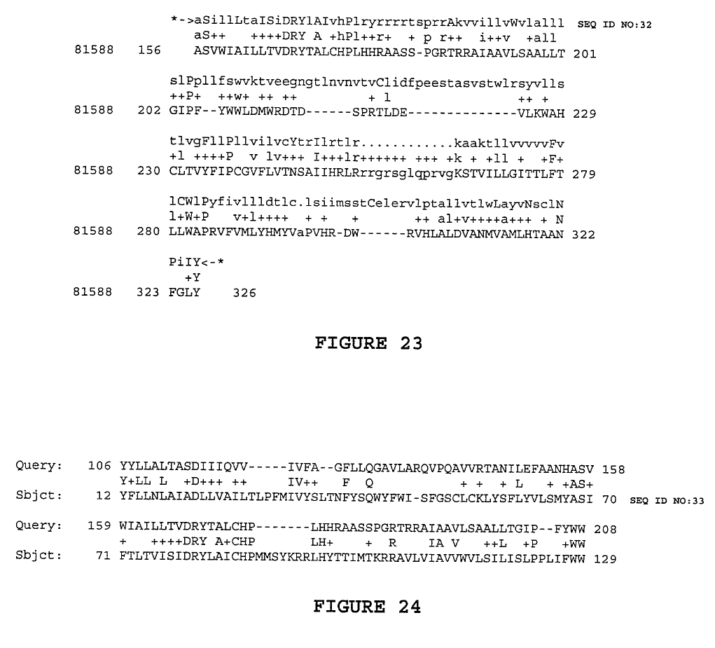

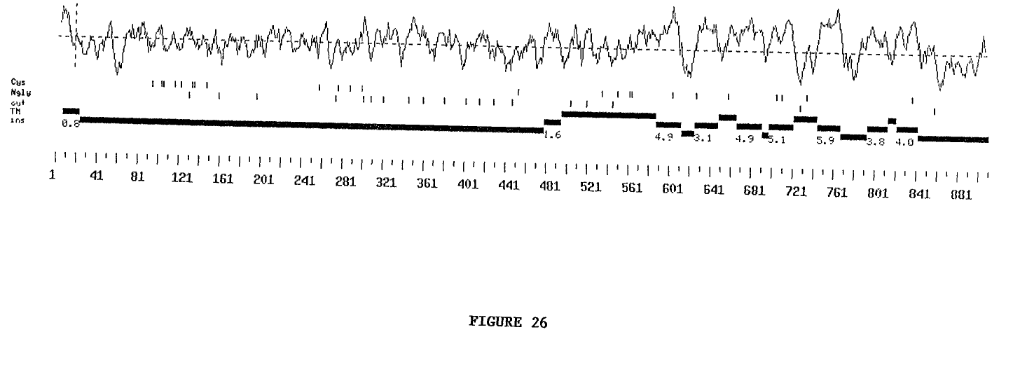

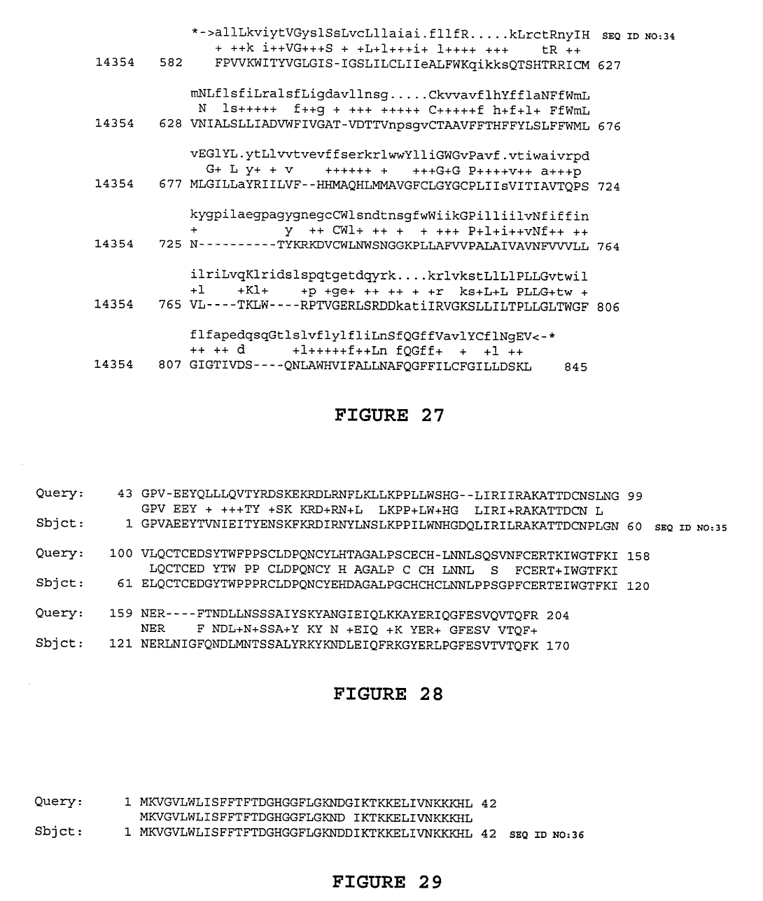

In a preferred embodiment, a 5495, 65507, 81588, or 14354 polypeptide or protein has at least one, two, three, four, five, six, seven, eight, or nine “transmembrane domains” or regions which includes at least about 12 to 35 more preferably about 14 to 30 or 15 to 25 amino acid residues and has at least about 60%, 70% 80% 90% 95%, 99%, or 100% homology with a “transmembrane domain,” e.g., the transmembrane domains of human 5495, 65507, 81588, or 14354 (e.g., residues 31-55, 66-90, 104-123, 145-163, 185-207, and 221-241 or 19-43, 57-76, 98-116, 138-160, and 174-194 of the mature form of SEQ ID NO:106-124, 148-166, 184-208, 228-250, and 272-288 or 9-31, 50-68, 92-110, 128-152, 172-194, and 216-232 of the mature form of SEQ ID NO: 11; 69-93, 105-129, 148-166, 190-209, 227-249, 269-291, and 313-329 of SEQ ID NO:14; 7-23, 478-494, 587-611, 625-647, 666-690, 697-720, 744-766, 793-812, and 821-841 or 328-344, 461-477, 570-594, 608-630, 649-673, 680-703,727-749, 776-795, and 804-824 of the mature form of SEQ ID NO:17) The transmembrane domain of human 5495, 65507, 81588, or 14354 is visualized in the hydropathy plots (FIGS. 14, 18, 22, and 26) as regions of about 15 to 25 amino acids where the hydropathy trace is mostly above the horizontal line.

A 5495, 65507, 81588, or 14354 polypeptide can include at least one, two, three, four, five, six, seven, eight, nine, or ten “non-transmembrane regions.” As used herein, the term “non-transmembrane region” includes an amino acid sequence not identified as a transmembrane domain. The non-transmembrane regions in 5495, 65507, 81588, or 14354 are located at about amino acids 1-30, 56-65, 91-103, 124-144, 164-184, 208-220, and 242-330 or 1-18, 44-56, 77-97,117-137,161-173, and 195-283 of the mature form of SEQ ID NO:8; 1-26,52-64,88-105, 125-147, 167-183, 209-227, 251-271, and 289-353 or 1-8, 32-49, 69-91, 111-127, 153-171, 195-215, and 233-297 of the mature form of SEQ ID NO:11; 1-68, 94-104, 130-147, 167-189,210-226, 250-268, 292-312, and 330-374 of SEQ ID NO: 14; 1-6, 24-477,495-586, 612-624, 648-665, 691-696, 721-743, 767-792, 813-820, and 842-910 or 1-327, 345-460, 478-569, 595-607,631-648, 674-679, 704-726, 750-775, 796-803, and 825-893 of the mature form of SEQ ID NO:17.

The non-transmembrane regions of 5495, 65507, 81588, or 14354 include at least one, two, three, four, or five cytoplasmic regions. When located at the N-terminus, the cytoplasmic region is referred to herein as the “N-terminal cytoplasmic domain.” As used herein, an “N-terminal cytoplasmic domain” includes an amino acid sequence having about 1 to 30, preferably about 1 to 25, more preferably about 1 to 20, or even more preferably about 1 to 18 amino acid residues in length and is located inside of a cell or within the cytoplasm of a cell. The C-terminal amino acid residue of an “N-terminal cytoplasmic domain” is adjacent to an N-terminal amino acid residue of a transmembrane domain in a 5495 or 65507 protein. For example, an N-terminal cytoplasmic domain is located at about amino acid residues 1 to 18 of the mature form of SEQ ID NO:8 or 1-8 of the mature form of SEQ ID NO:11.

In a preferred embodiment, a polypeptide or protein has an N-terminal cytoplasmic domain or a region which includes at least about 5, preferably about 1 to 30, and more preferably about 1 to 20 amino acid residues and has at least about 60%, 70% 80% 90% 95%, 99%, or 100% homology with an “N-terminal cytoplasmic domain,” e.g., the N-terminal cytoplasmic domain of human 5495 or 65507 (e.g., residues 1 to 18 of the mature form of SEQ ID NO:8 or 1-8 of the mature form of SEQ ID NO:11).

In another embodiment, a cytoplasmic region of a 65507, 81588, or 14354 protein can include the C-terminus and can be a “C-terminal cytoplasmic domain,” also referred to herein as a “C-terminal cytoplasmic tail.” As used herein, a “C-terminal cytoplasmic domain” includes an amino acid sequence having a length of at least about 20, preferably about 30 to 80, more preferably about 40 to 70 amino acid residues and is located inside of a cell or within the cytoplasm of a cell. The N-terminal amino acid residue of a “C-terminal cytoplasmic domain” is adjacent to a C-terminal amino acid residue of a transmembrane domain in a 65507, 81588, or 14354 protein. For example, a C-terminal cytoplasmic domain is located at about amino acid residues 289-353 or 233-297 of the mature form of SEQ ID NO:11; 330-374 of SEQ ID NO:14; 842-910 or 825-893 of the mature form of SEQ ID NO:17.

In a preferred embodiment, a 65507, 81588, or 14354 polypeptide or protein has a C-terminal cytoplasmic domain or a region which includes at least about 5, preferably about 30 to 80, and more preferably about 40 to 70 amino acid residues and has at least about 60%, 70% 80% 90% 95%, 99%, or 100% homology with a C-terminal cytoplasmic domain,” e.g., the C-terminal cytoplasmic domain of human 65507, 81588, or 14354 (e.g., residues 289-353 or 233-297 of the mature form of SEQ ID NO:11; 330-374 of SEQ ID NO:14; 842-910 or 825-893 of the mature form of SEQ ID NO:17).

In another embodiment, a 5495, 65507, 81588, or 14354 protein includes at least one, two, three, or four cytoplasmic loops. As used herein, the term “loop” includes an amino acid sequence that resides outside of a phospholipid membrane, having a length of at least about 4, preferably about 5 to 500, more preferably about 6 to 475 amino acid residues, and has an amino acid sequence that connects two transmembrane domains within a protein or polypeptide. Accordingly, the N-terminal amino acid of a loop is adjacent to a C-terminal amino acid of a transmembrane domain in a 5495, 65507, 81588, or 14354 molecule, and the C-terminal amino acid of a loop is adjacent to an N-terminal amino acid of a transmembrane domain in a 5495, 65507, 81588, or 14354 molecule. As used herein, a “cytoplasmic loop” includes a loop located inside of a cell or within the cytoplasm of a cell. For example, a “cytoplasmic loop” can be found at about amino acid residues 56-65, 124-144, and 208-220 or 77-97 and 161-173 of the mature form of SEQ ID NO:8; 52-64, 125-147, and 209-227 or 69-91 and 153-171 of the mature form of SEQ ID NO:l 1; 94-104, 167-189, and 250-268 of SEQ ID NO:14; 24-477, 612-624, 691-696, and 767-792 or 345-460, 595-607, 674-679 and 750-775 of the mature form of SEQ ID NO:17.

In a preferred embodiment, a 5495, 65507, 81588, or 14354 polypeptide or protein has a cytoplasmic loop or a region which includes at least about 4, preferably about 5 to 500, and more preferably about 6 to 475 amino acid residues and has at least about 60%, 70% 80% 90% 95%, 99%, or 100% homology with a cytoplasmic loop,” e.g., a cytoplasmic loop of human 5495, 65507, 81588, or 14354 (e.g., residues 56-65, 124-144, and 208-220 or 77-97 and 161-173 of the mature form of SEQ ID NO:8; 52-64, 125-147, and 209-227 or 69-91 and 153-171 of the mature form of SEQ ID NO:11; 94-104, 167-189, and 250-268 of SEQ ID NO:14; 24-477, 612-624, 691-696, and 767-792 or 345-460, 595-607, 674-679 and 750-775 of the mature form of SEQ ID NO:17).

In another embodiment, a 5495, 65507, 81588, or 14354 protein includes at least one, two, three or four non-cytoplasmic loops. As used herein, a “non-cytoplasmic loop” includes an amino acid sequence located outside of a cell or within an intracellular organelle. Non-cytoplasmic loops include extracellular domains (i.e., outside of the cell) and intracellular domains (i.e., within the cell). When referring to membrane-bound proteins found in intracellular organelles (e.g., mitochondria, endoplasmic reticulum, peroxisomes microsomes, vesicles, endosomes, and lysosomes), non-cytoplasmic loops include those domains of the protein that reside in the lumen of the organelle or the matrix or the intermembrane space. For example, a “non-cytoplasmic loop” can be found at about amino acid residues 91-103 and 164-184 or 44-56 and 117-137 of the mature form of SEQ ID NO:8; 88-105, 167-183, and 251-271 or 32-49, 111-127 and 195-215 of the mature form of SEQ ID NO:11; 130-147, 210-226 and 292-312 of SEQ ID NO:14; 495-586, 648-665, 721-743 and 813-820 or 478-569, 631-648, 704-726 and 796-803 of the mature form of SEQ ID NO:17.

In a preferred embodiment, a 5495, 65507, 81588, or 14354 polypeptide or protein has at least one non-cytoplasmic loop or a region which includes at least about 4, preferably about 5 to 150, more preferably about 6 to 100 amino acid residues and has at least about 60%, 70% 80% 90% 95%, 99%, or 100% homology with a “non-cytoplasmic loop,” e.g., at least one non-cytoplasmic loop of human 5495, 65507, 81588, or 14354 (e.g., residues 91-103 and 164-184 or 44-56 and 117-137 of the mature form of SEQ ID NO:8; 88-105, 167-183, and 251-271 or 32-49, 111-127 and 195-215 of the mature form of SEQ ID NO:11; 130-147, 210-226 and 292-312 of SEQ ID NO:14; 495-586, 648-665, 721-743 and 813-820 or 478-569, 631-648, 704-726 and 796-803 of the mature form of SEQ ID NO:17).

In a preferred embodiment, a 5495 family member can include at least one seven transmembrane receptor family domain (PFAM Accession Number PF00001). Furthermore, a 5495 family member can include at least one, and preferably two protein kinase C phosphorylation sites (PS00005); at least one, and preferably two casein kinase II phosphorylation sites (PS00006); at least one, two, three, four, five, and preferably six N-myristoylation (PS00008); at least one, and preferably two leucine zipper pattern sites (PS00029); and at least one G-protein coupled receptors signature site (PS00237).

In another preferred embodiment, a 65507 family member can include at least one seven transmembrane receptor family domain (PFAM Accession Number PF00001). Furthermore, a 65507 family member can include at least one, and preferably two N-glycosylation sites (PS00001); at least one cAMP-and cGMP-dependent protein kinase phosphorylation sites (PS00004); at least one, two, three, and preferably four protein kinase C phosphorylation sites (PS00005); and at least one, and preferably two N-myristoylation sites (PS00008).

In another preferred embodiment, an 81588 family member can include at least one seven transmembrane receptor family domains (PFAM Accession Number PF00001). Furthermore, an 81588 family member can include at least one N-glycosylation sites (PS00001); at least one cAMP-and cGMP-dependent protein kinase phosphorylation sites (PS00004); at least one, two, three, four, and preferably five protein kinase C phosphorylation sites (PS00005); at least one, two, three, four, and preferably five casein kinase II phosphorylation sites (PS00006); at least one, two, and preferably three N-myristoylation sites (PS00008); and at least one G-protein coupled receptors signature site (PS00237).

In another preferred embodiment, a 14354 family member can include at least one seven transmembrane receptor family domains (PFAM Accession Number PF00002). Furthermore, a 14354 family member can include at least one, two, three, four, five, six, seven, eight, nine, ten, eleven, twelve, thirteen, fourteen, fifteen, sixteen, seventeen, eighteen, and preferably nineteen N-glycosylation sites (PS00001); at least one, two, three, four, five, six, seven, eight, nine, ten, eleven, and preferably twelve protein kinase C phosphorylation sites (PS00005); at least one, two, three, four, five, six, seven, eight, nine, ten, eleven, twelve, thirteen, fourteen, fifteen, sixteen, and preferably seventeen casein kinase II phosphorylation sites (PS00006); at least one, two, three, four, five, six, seven, eight, nine, ten, eleven, and preferably twelve N-myristoylation sites (PS00008); and at least one prokaryotic membrane lipoprotein lipid attachment site (PS00013).

As the 5495 polypeptides of the invention may modulate 5495-mediated activities, they may be useful for developing novel diagnostic and therapeutic agents for 5495-mediated or related disorders, as described below. Likewise, as the 65507 polypeptides of the invention may modulate 65507-mediated activities, they may be useful for developing novel diagnostic and therapeutic agents for 65507-mediated or related disorders, as described below. Likewise, as the 81588 polypeptides of the invention may modulate 81588-mediated activities, they may be useful for developing novel diagnostic and therapeutic agents for 81588-mediated or related disorders, as described below. Likewise, as the 14354 polypeptides of the invention may modulate 14354-mediated activities, they may be useful for developing novel diagnostic and therapeutic agents for 14354-mediated or related disorders, as described below.

Based on the above-described sequence similarities, the 5495, 65507, 81588 and 14354 molecules of the present invention are predicted to have similar biological activities as seven transmembrane proteins such as G-protein coupled receptor family members. Thus, the 5495, 65507, 81588 and 14354 molecules can act as novel diagnostic targets and therapeutic agents for controlling one or more of cellular proliferative and/or differentiative disorders, immune disorders, heart disorders, cardiovascular disorders, including endothelial cell disorders, hematopoietic disorders, blood vessel disorders, brain disorders, pain and metabolic disorders, hormonal disorders, liver disorders, and platelet disorders.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

The 38650, 28472, 5495, 65507, 81588 or 14354 nucleic acid and protein of the invention can be used to treat and/or diagnose a variety of proliferative disorders. E.g., such disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L., (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Disorders involving the immune system include autoimmune disorders or immune deficiency disorders, e.g., congenital X-linked infantile hypogammaglobulinemia, transient hypogammaglobulinemia, common variable immunodeficiency, selective IgA deficiency, chronic mucocutaneous candidiasis, or severe combined immunodeficiency.

Examples of disorders involving the heart or “cardiovascular disorder” include, but are not limited to, a disease, disorder, or state involving the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. Examples of cardiovascular disorders include but are not limited to, hypertension, atherosclerosis, coronary artery spasm, coronary artery disease, arrhythmias, heart failure, including but not limited to, cardiac hypertrophy, left-sided heart failure, and right-sided heart failure; ischemic heart disease, including but not limited to angina pectoris, myocardial infarction, chronic ischemic heart disease, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy, hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; neoplastic heart disease, including but not limited to, primary cardiac tumors, such as myxoma, lipoma, papillary fibroelastoma, rhabdomyoma, and sarcoma, and cardiac effects of noncardiac neoplasms; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia, disorders involving cardiac transplantation, and congestive heart failure.

A cardiovasular disease or disorder also includes an endothelial cell disorder. As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Examples of hematopoietic disorders include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

Disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

Disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyclia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicella-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fingal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degenration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

Diseases of metabolic imbalance include, but are not limited to, obesity, anorexia nervosa, cachexia, lipid disorders, and diabetes. Examples of pain disorders include, but are not limited to, pain response elicited during various forms of tissue injury, e.g., inflammation, infection, and ischemia, usually referred to as hyperalgesia (described in, for example, Fields, H. L., (1987) Pain, New York:McGraw-Hill); pain associated with muscoloskeletal disorders, e.g., joint pain; tooth pain; headaches; pain associated with surgery; pain related to irritable bowel syndrome; or chest pain.

Disorders of the present invention also include hormonal disorders, such as conditions or diseases in which the production and/or regulation of hormones in an organism is aberrant. Examples of such disorders and diseases include type I and type II diabetes mellitus, pituitary disorders (e.g., growth disorders), thyroid disorders (e.g., hypothyroidism or hyperthyroidism), and reproductive or fertility disorders (e.g., disorders which affect the organs of the reproductive system, e.g., the prostate gland, the uterus, or the vagina; disorders which involve an imbalance in the levels of a reproductive hormone in a subject; disorders affecting the ability of a subject to reproduce; and disorders affecting secondary sex characteristic development, e.g., adrenal hyperplasia).

Disorders which may be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolsim, for example, fibrosis resulting from a storage disorder such as Gaucher's disease (lipid abnormalities) or a glycogen storage disease, A1-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein may be useful for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

The 38650, 28472, 5495, 65507, 81588 or 14354 protein, fragments thereof, and derivatives and other variants of the sequence in SEQ ID NO:2,5,8,11, 14 or 17 are collectively referred to as “polypeptides or proteins of the invention” or “38650, 28472, 5495, 65507, 81588 or 14354 polypeptides or proteins”. Nucleic acid molecules encoding such polypeptides or proteins are collectively referred to as “nucleic acids of the invention” or “38650, 28472, 5495, 65507, 81588 or 14354 nucleic acids.” 38650, 28472, 5495, 65507, 81588 or 14354 molecules refer to 38650, 28472, 5495, 65507, 81588 or 14354 nucleic acids, polypeptides, and antibodies.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under low stringency, medium stringency, high stringency, or very high stringency conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. A preferred, example of stringent hybridization conditions are hybridization in 6×sodium chloride/sodium citrate (SSC) at about 45° C., followed by one or more washes in 0.2×SSC, 0.1% SDS at 50° C. Another example of stringent hybridization conditions are hybridization in 6×sodium chloride/sodium citrate (SSC) at about 45° C., followed by one or more washes in 0.2×SSC, 0.1% SDS at 55° C. A further example of stringent hybridization conditions are hybridization in 6×sodium chloride/sodium citrate (SSC) at about 45° C., followed by one or more washes in 0.2×SSC, 0.1% SDS at 60° C. Preferably, stringent hybridization conditions are hybridization in 6×sodium chloride/sodium citrate (SSC) at about 45° C., followed by one or more washes in 0.2×SSC, 0.1% SDS at 65° C. Particularly preferred stringency conditions (and the conditions that should be used if the practitioner is uncertain about what conditions should be applied to determine if a molecule is within a hybridization limitation of the invention) are 0.5M Sodium Phosphate, 7% SDS at 65° C., followed by one or more washes at 0.2×SSC, 1% SDS at 65° C. Preferably, an isolated nucleic acid molecule of the invention that hybridizes under stringent conditions to the sequence of SEQ ID NO:1, 3, 4, 6, 7, 9, 10, 12, 13, 15, 16 or 18, corresponds to a naturally-occurring nucleic acid molecule.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules which include an open reading frame encoding a 38650, 28472, 5495, 65507, 81588 or 14354 protein, preferably a mammalian 38650, 28472, 5495, 65507, 81588 or 14354 protein, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of 38650, 28472, 5495, 65507, 81588 or 14354 protein having less than about 30%, 20%, 10% and more preferably 5% (by dry weight), of non-38650, 28472, 5495, 65507, 81588 or 14354 protein (also referred to herein as a “contaminating protein”), or of chemical precursors or non-38650, 28472, 5495, 65507, 81588 or 14354 chemicals. When the 38650, 28472, 5495, 65507, 81588 or 14354 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of 38650, 28472, 5495, 65507, 81588 or 14354(e.g., the sequence of SEQ ID NO:1, 3, 4, 6, 7, 9, 10, 12, 13, 15, 16 or 18, or the nucleotide sequence of the DNA insert of the plasmid deposited with ATCC as Accession Number______) without abolishing or more preferably, without substantially altering a biological activity, whereas an “essential” amino acid residue results in such a change. For example, amino acid residues that are conserved among the polypeptides of the present invention, e.g., those present in the adenosine deaminase, glycoprotease, or 7TM receptor domains, are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 38650, 28472, 5495, 65507, 81588 or 14354 protein is preferably replaced with another amino acid residue from the same side chain family. Alternatively, in another embodiment, mutations can be introduced randomly along all or part of a 38650, 28472, 5495, 65507, 81588 or 14354 coding sequence, such as by saturation mutagenesis, and the resultant mutants can be screened for 38650, 28472, 5495, 65507, 81588 or 14354 biological activity to identify mutants that retain activity. Following mutagenesis of SEQ ID NO:1, 3, 4, 6, 7, 9, 10, 12, 13, 15, 16 or 18, or the nucleotide sequence of the DNA insert of the plasmid deposited with ATCC as Accession Number______, the encoded protein can be expressed recombinantly and the activity of the protein can be determined.

As used herein, a “biologically active portion” of a 38650, 28472, 5495, 65507, 81588 or 14354 protein includes a fragment of a 38650, 28472, 5495, 65507, 81588 or 14354 protein which participates in an interaction between a 38650, 28472, 5495, 65507, 81588 or 14354 molecule and a non-38650, 28472, 5495, 65507, 81588 or 14354 molecule. Biologically active portions of a 38650, 28472, 5495, 65507, 81588 or 14354 protein include peptides comprising amino acid sequences sufficiently homologous to or derived from the amino acid sequence of the 38650, 28472, 5495, 65507, 81588 or 14354 protein, e.g., the amino acid sequence shown in SEQ ID NO:2, 5, 8, 11, 14 or 17, which include less amino acids than the full length 38650, 28472, 5495, 65507, 81588 or 14354 proteins, and exhibit at least one activity of a 38650, 28472, 5495, 65507, 81588 or 14354 protein. Typically, biologically active portions comprise a domain or motif with at least one activity of the 38650, 28472, 5495, 65507, 81588 or 14354 protein, e.g., adenosine deaminase, glycoprotease, or 7TM receptor domain activity. A biologically active portion of a 38650, 28472, 5495, 65507, 81588 or 14354 protein can be a polypeptide which is, for example, 10, 25, 50, 100, 200 or more amino acids in length. Biologically active portions of a 38650, 28472, 5495, 65507, 81588 or 14354 protein can be used as targets for developing agents which modulate a 38650, 28472, 5495, 65507, 81588 or 14354 mediated activity, e.g., adenosine deaminase, glycoprotease, or 7TM receptor domain activity.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence (e.g., when aligning a second sequence to the 38650 amino acid sequence of SEQ ID NO:2 having 355 amino acid residues, at least 107, preferably at least 142, more preferably at least 178, even more preferably at least 213, and even more preferably at least 249, 284, 320 or 355 amino acid residues are aligned, or when aligning a second sequence to the 28472 amino acid sequence of SEQ ID NO:5 having 414 amino acid residues, at least 124, preferably at least 166, more preferably at least 207, even more preferably at least 248, and even more preferably at least 290, 331, 373 or 414 amino acid residues are aligned, or when aligning a second sequence to the 5495 amino acid sequence of SEQ ID NO:8 having 330 amino acid residues, at least 99, preferably at least 132, more preferably at least 165, even more preferably at least 198, and even more preferably at least 231, 264, 297 or 330 amino acid residues are aligned, or when aligning a second sequence to the 65507 amino acid sequence of SEQ ID NO:11 having 353 amino acid residues, at least 106, preferably at least 141, more preferably at least 177, even more preferably at least 212, and even more preferably at least 247, 282, 318 or 353 amino acid residues are aligned, or when aligning a second sequence to the 81588 amino acid sequence of SEQ ID NO:14 having 374 amino acid residues, at least 112, preferably at least 150, more preferably at least 187, even more preferably at least 224, and even more preferably at least 262, 299, 337 or 374 amino acid residues are aligned, or when aligning a second sequence to the 14354 amino acid sequence of SEQ ID NO:17 having 910 amino acid residues, at least 273, preferably at least 364, more preferably at least 455, even more preferably at least 546, and even more preferably at least 637, 728, 819 or 910 amino acid residues are aligned.. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology” ). The percent identity between the two sequences is a function of the number of identical positions shared by the sequences, taking into account the number of gaps, and the length of each gap, which need to be introduced for optimal alignment of the. two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) is using a Blossum 62 scoring matrix with a gap open penalty of 12, a gap extend penalty of 4, and a frameshift gap penalty of 5.

The percent identity between two amino acid or nucleotide sequences can be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990) J. Mol. Biol. 215:403-10. BLAST nucleotide searches can be performed with the NBLAST program, score=100, wordlength=12 to obtain nucleotide sequences homologous to 38650, 28472, 5495, 65507, 81588 or 14354 nucleic acid molecules of the invention. BLAST protein searches can be performed with the XBLAST program, score=50, wordlength=3 to obtain amino acid sequences homologous to 38650, 28472, 5495, 65507, 81588 or 14354 protein molecules of the invention. To obtain gapped alignments for comparison purposes, Gapped BLAST can be utilized as described in Altschul et al., (1997) Nucleic Acids Res. 25 (17):3389-3402. When utilizing BLAST and Gapped BLAST programs, the default parameters of the respective programs (e.g., XBLAST and NBLAST) can be used. See http://www.ncbi.nlm.nih.gov.

Particular 38650, 28472, 5495, 65507, 81588 or 14354 polypeptides of the present invention have an amino acid sequence substantially identical to the amino acid sequence of SEQ ID NO:2, 5, 8, 11, 14 or 17. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94% 95%, 96%, 97%, 98% or 99% identity to SEQ ID NO:2, 5, 8, 11, 14 or 17 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97%, 98% or identity to SEQ ID NO:1, 3, 4, 6, 7, 9, 10, 12, 13, 15, 16 or 18 are termed substantially identical.

“Misexpression or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject”, as used herein, can refer to a mammal, e.g., a human, or to an experimental or animal or disease model. The subject can also be a non-human animal, e.g., a horse, cow, goat, or other domestic animal.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

Various aspects of the invention are described in further detail below.

Isolated Nucleic Acid Molecules

In one aspect, the invention provides, an isolated or purified, nucleic acid molecule that encodes a 38650, 28472, 5495, 65507, 81588 or 14354 polypeptide described herein, e.g., a full length 38650, 28472, 5495, 65507, 81588 or 14354 protein or a fragment thereof, e.g., a biologically active portion of 38650, 28472, 5495, 65507, 81588 or 14354 protein. Also included is a nucleic acid fragment suitable for use as a hybridization probe, which can be used, e.g., to identify a nucleic acid molecule encoding a polypeptide of the invention, 38650, 28472, 5495, 65507, 81588 or 14354 mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO:1, 3, 4, 6, 7, 9, 10, 12, 13, 15, 16 or 18, or the nucleotide sequence of the DNA insert of the plasmid deposited with ATCC as Accession Number______, or a portion of any of these nucleotide sequences. In one embodiment, the nucleic acid molecule includes sequences encoding the human 38650, 28472, 5495, 65507, 81588 or 14354 protein (i.e., “the coding region”, from nucleotides 340-1407 of SEQ ID NO:1, 146-1390 of SEQ ID NO:4, 138-1130 of SEQ ID NO:7, 139-1200 of SEQ ID NO:10, 97-1221 of SEQ ID NO:13, and 199-2931 of SEQ ID NO:16, including the terminal codon), as well as 5′ untranslated sequences (nucleotides 1-339 of SEQ ID NO:1, 1-145 of SEQ ID NO:4, 1-137 of SEQ ID NO:7, 1-138 of SEQ ID NO:10, 1-96 of SEQ ID NO:13, and 1-198 of SEQ ID NO:16). Alternatively, the nucleic acid molecule can include only the coding region of SEQ ID NO:1, 4, 7, 10, 13 or 16(e.g., nucleotides 340-1407 of SEQ ID NO:1, 146-1390 of SEQ ID NO:4, 138-1130 of SEQ ID NO:7, 139-1200 of SEQ ID NO:10, 97-1221 of SEQ ID NO:13, and 199-2931 of SEQ ID NO:16, corresponding to SEQ ID NO:3, 6, 9, 12, 15 and 18) and, e.g., no flanking sequences which normally accompany the subject sequence. In another embodiment, the nucleic acid molecule encodes a sequence corresponding to the mature protein of SEQ ID NO:2, 5, 8, 11, 14 or 17.

In another embodiment, an isolated nucleic acid molecule of the invention includes a nucleic acid molecule which is a complement of the nucleotide sequence shown in SEQ ID NO:1, 3, 4, 6, 7, 9, 10, 12, 13, 15, 16 or 18, or the nucleotide sequence of the DNA insert of the plasmid deposited with ATCC as Accession Number______, or a portion of any of these nucleotide sequences. In other embodiments, the nucleic acid molecule of the invention is sufficiently complementary to the nucleotide sequence shown in SEQ ID NO:1,3,4,6,7,9,10,12,13, 15, 16 or 18, or the nucleotide sequence of the DNA insert of the plasmid deposited with ATCC as Accession Number______ such that it can hybridize to the nucleotide sequence shown in SEQ ID NO:1, 3, 4, 6, 7, 9, 10, 12, 13, 15, 16 or 18, or the nucleotide sequence of the DNA insert of the plasmid deposited with ATCC as Accession Number______, thereby forming a stable duplex.

In one embodiment, an isolated nucleic acid molecule of the present invention includes a nucleotide sequence which is at least about 60%, 65%, 70%, 75%, 80%, 85%, 90%, 91%, 92%, 93%, 94%, 95%, 96%, 97%, 98%, 99%, or more homologous to the nucleotide sequence shown in SEQ ID NO:1, 3, 4, 6, 7, 9, 10, 12, 13, 15, 16 or 18, or the nucleotide sequence of the DNA insert of the plasmid deposited with ATCC as Accession Number______. In the case of an isolated nucleic acid molecule which is longer than or equivalent in length to the reference sequence, e.g., SEQ ID NO:1, 3, 4, 6, 7, 9, 10, 12, 13, 15, 16 or 18, the comparison is made with the full length of the reference sequence. Where the isolated nucleic acid molecule is shorter than the reference sequence, e.g., shorter than SEQ ID NO:1, 3, 4, 6, 7, 9, 10, 12, 13, 15, 16 or 18, the comparison is made to a segment of the reference sequence of the same length (excluding any loop required by the homology calculation).

38650, 28472, 5495 65507, 81588 or 14354 Nucleic Acid Fragments

A nucleic acid molecule of the invention can include only a portion of the nucleic acid sequence of SEQ ID NO:1, 3, 4, 6, 7, 9, 10, 12, 13, 15, 16 or 18, or the nucleotide sequence of the DNA insert of the plasmid deposited with ATCC as Accession Number______. For example, such a nucleic acid molecule can include a fragment which can be used as a probe or primer or a fragment encoding a portion of a 38650, 28472, 5495, 65507, 81588 or 14354 protein, e.g., an immunogenic or biologically active portion of a 38650, 28472, 5495, 65507, 81588 or 14354 protein. A fragment can comprise: nucleotides 364-1371 of SEQ ID NO:1, 254-1261 of SEQ ID NO:4, 276-974 of SEQ ID NO:7, 265-993 of SEQ ID NO:10, 562-1074 of SEQ ID NO:13, or 1942-2733 of SEQ ID NO:16, which encodes an adenosine deaminase, glycoprotease, or 7TM receptor domain of human 38650, 28472, 5495, 65507, 81588 or 14354. The nucleotide sequence determined from the cloning of the 38650, 28472, 5495, 65507, 81588 or 14354 gene allows for the generation of probes and primers designed for use in identifying and/or cloning other 38650, 28472, 5495, 65507, 81588 or 14354 family members, or fragments thereof, as well as 38650, 28472, 5495, 65507, 81588 or 14354 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 150 amino acids in length. Fragments also include nucleic acid sequences corresponding to specific amino acid sequences described above or fragments thereof. Nucleic acid fragments should not be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, the nucleic acid fragment can include an adenosine deaminase, glycoprotease, or 7TM receptor domain. In a preferred embodiment the fragment is at least, 50, 100, 200, 300, 400, 500, 600, 700, or 900 base pairs in length.

38650, 28472, 5495, 65507, 81588 or 14354 probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of SEQ ID NO:1, 3, 4, 6, 7, 9, 10, 12, 13, 15, 16 or 18, or the nucleotide sequence of the DNA insert of the plasmid deposited with ATCC as Accession Number______, or of a naturally occurring allelic variant or mutant of SEQ ID NO:1, 3, 4, 6, 7, 9, 10, 12, 13, 15, 16 or 18, or the nucleotide sequence of the DNA insert of the plasmid deposited with ATCC as Accession Number______.

In a preferred embodiment the nucleic acid is a probe which is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes a adenosine deaminase, glycoprotease, or 7TM receptor domain (e.g., about amino acid residues 9-344 of SEQ ID NO:2, 37-372 of SEQ ID NO:5, 47-279 of SEQ ID NO:8, 43-285 of SEQ ID NO:11, 156-326 of SEQ ID NO:14, or 582-845 of SEQ ID NO:17).

In another embodiment a set of primers is provided, e.g., primers suitable for use in a PCR, which can be used to amplify a selected region of a 38650, 28472, 5495, 65507, 81588 or 14354 sequence, e.g., a region described herein. The primers should be at least 5, 10, or 50 base pairs in length and less than 100, or less than 200, base pairs in length. The primers should be identical, or differs by one base from a sequence disclosed herein or from a naturally occurring variant. E.g., primers suitable for amplifying all or a portion of any of the following regions are provided: a adenosine deaminase, glycoprotease, or 7TM receptor domain (e.g., about amino acid residues 9-344 of SEQ ID NO:2, 37-372 of SEQ ID NO:5, 47-279 of SEQ ID NO:8, 43-285 of SEQ ID NO:l 1, 156-326 of SEQ ID NO:14, or 582-845 of SEQ ID NO:17).

A nucleic acid fragment can encode an epitope bearing region of a polypeptide described herein.

A nucleic acid fragment encoding a “biologically active portion of a 38650, 28472, 5495, 65507, 81588 or 14354 polypeptide” can be prepared by isolating a portion of the nucleotide sequence of SEQ ID NO:1, 3, 4, 6, 7, 9, 10, 12, 13, 15, 16 or 18, or the nucleotide sequence of the DNA insert of the plasmid deposited with ATCC as Accession Number______, which encodes a polypeptide having a 38650, 28472, 5495, 65507, 81588 or 14354 biological activity (e.g., the biological activities of the 38650, 28472, 5495, 65507, 81588 or 14354 proteins as described herein), expressing the encoded portion of the 38650, 28472, 5495, 65507, 81588 or 14354 protein (e.g., by recombinant expression in vitro) and assessing the activity of the encoded portion of the 38650, 28472, 5495, 65507, 81588 or 14354 protein. For example, a nucleic acid fragment encoding a biologically active portion of 38650, 28472, 5495, 65507, 81588 or 14354 includes a adenosine deaminase, glycoprotease, or 7TM receptor domain (e.g., about amino acid residues 9-344 of SEQ ID NO:2, 37-372 of SEQ ID NO:5, 47-279 of SEQ ID NO:8, 43-285 of SEQ IDNO:11, 156-326 of SEQ ID NO:14, or 582-845 of SEQ ID NO:17). A nucleic acid fragment encoding a biologically active portion of a 38650, 28472, 5495, 65507, 81588 or 14354 polypeptide, may comprise a nucleotide sequence which is greater than 300-1200 or more nucleotides in length.

In preferred embodiments, nucleic acids include a nucleotide sequence which is about 300, 400, 500, 600, 700, 800, 900, 1000, 1100, 1200, 1300, 1400 nucleotides in length and hybridizes under stringent hybridization conditions to a nucleic acid molecule of SEQ ID NO:1, 3, 4, 6, 7, 9, 10, 12, 13, 15, 16 or 18, or the nucleotide sequence of the DNA insert of the plasmid deposited with ATCC as Accession Number______.

38650, 28472, 5495, 65507, 81588 or 14354 Nucleic Acid Variants

The invention further encompasses nucleic acid molecules that differ from the nucleotide sequence shown in SEQ ID NO:1, 3, 4, 6, 7, 9, 10, 12, 13, 15, 16 or 18, or the nucleotide sequence of the DNA insert of the plasmid deposited with ATCC as Accession Number______. Such differences can be due to degeneracy of the genetic code (and result in a nucleic acid which encodes the same 38650, 28472, 5495, 65507, 81588 or 14354 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:2, 5, 8, 11, 14 or 17. If alignment is needed for this comparison the sequences should be aligned for maximum homology. “Looped” out sequences from deletions or insertions, or mismatches, are considered differences.

Nucleic acids of the inventor can be chosen for having codons, which are preferred, or non preferred, for a particular expression system. E.g., the nucleic acid can be one in which at least one colon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO:1, 3, 4, 6, 7, 9, 10, 12, 13, 15, 16 or 18, or the nucleotide sequence of the DNA insert of the plasmid deposited with ATCC as Accession Number______, e.g., as follows: by at least one but less than 10, 20, 30, or 40 nucleotides; at least one but less than 1%, 5%, 10% or 20% of the in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the amino acid sequence shown in SEQ ID NO:2, 5, 8, 11, 14 or 17 or a fragment of this sequence. Such nucleic acid molecules can readily be obtained as being able to hybridize under stringent conditions, to the nucleotide sequence shown in SEQ ID NO:3, 6, 9, 12, 15, or 18 or a fragment of this sequence. Nucleic acid molecules corresponding to orthologs, homologs, and allelic variants of the 38650, 28472, 5495, 65507, 81588 or 14354 cDNAs of the invention can further be isolated by mapping to the same chromosome or locus as the 38650, 28472, 5495, 65507, 81588 or 14354 gene. Preferred variants include those that are correlated with adenosine deaminase, glycoprotease, or 7TM receptor activity.

Allelic variants of 38650, 28472, 5495, 65507, 81588 or 14354, e.g., human 38650, 28472, 5495, 65507, 81588 or 14354, include both functional and non-functional proteins. Functional allelic variants are naturally occurring amino acid sequence variants of the 38650, 28472, 5495, 65507, 81588 or 14354 protein within a population that maintain the ability to modulate the phosphorylation state of itself or another protein or polypeptide. Functional allelic variants will typically contain only conservative substitution of one or more amino acids of SEQ ID NO:2, 5, 8, 11, 14 or 17, or substitution, deletion or insertion of non-critical residues in non-critical regions of the protein. Non-functional allelic variants are naturally-occurring amino acid sequence variants of the 38650, 28472, 5495, 65507, 81588 or 14354, e.g., human 38650, 28472, 5495, 65507, 81588 or 14354, protein within a population that do not have the ability to attach an acyl chain to a lipid precursor. Non-functional allelic variants will typically contain a non-conservative substitution, a deletion, or insertion, or premature truncation of the amino acid sequence of SEQ ID NO:2, 5, 8, 11, 14 or 17, or a substitution, insertion, or deletion in critical residues or critical regions of the protein.

Moreover, nucleic acid molecules encoding other 38650, 28472, 5495, 65507, 81588 or 14354 family members and, thus, which have a nucleotide sequence which differs from the 38650, 28472, 5495, 65507, 81588 or 14354 sequences of SEQ ID NO:1, 3, 4, 6, 7, 9, 10, 12, 13, 15, 16 or 18, or the nucleotide sequence of the DNA insert of the plasmid deposited with ATCC as Accession Number______ are intended to be within the scope of the invention.

Antisense Nucleic Acid Molecules, Ribozymes and Modified 38650, 28472. 5495, 65507, 81588 or 14354 Nucleic Acid Molecules

In another aspect, the invention features, an isolated nucleic acid molecule which is antisense to 38650, 28472, 5495, 65507, 81588 or 14354. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire 38650, 28472, 5495, 65507, 81588 or 14354 coding strand, or to only a portion thereof (e.g., the coding region of human 38650, 28472, 5495, 65507, 81588 or 14354 corresponding to SEQ ID NO:3). In another embodiment, the antisense nucleic acid molecule is antisense to a “noncoding region” of the coding strand of a nucleotide sequence encoding 38650, 28472, 5495, 65507, 81588 or 14354 (e.g., the 5′ and 3′ untranslated regions).

An antisense nucleic acid can be designed such that it is complementary to the entire coding region of 38650, 28472, 5495, 65507, 81588 or 14354 mRNA, but more preferably is an oligonucleotide which is antisense to only a portion of the coding or noncoding region of 38650, 28472, 5495, 65507, 81588 or 14354 mRNA. For example, the antisense oligonucleotide can be complementary to the region surrounding the translation start site of 38650, 28472, 5495, 65507, 81588 or 14354 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a 38650, 28472, 5495, 65507, 81588 or 14354 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., (1987) Nucleic Acids. Res. 15:6625-6641). The antisense nucleic acid molecule can also comprise a 2′-o-methylribonucleotide (Inoue et al., (1987) Nucleic Acids Res. 15:6131-6148) or a chimeric RNA-DNA analogue (Inoue et al., (1987)FEBS Lett. 215:327-330).

In still another embodiment, an antisense nucleic acid of the invention is a ribozyme. A ribozyme having specificity for a 38650, 28472, 5495, 65507, 81588 or 14354-encoding nucleic acid can include one or more sequences complementary to the nucleotide sequence of a 38650, 28472, 5495, 65507, 81588 or 14354 cDNA disclosed herein (i.e., SEQ ID NO:1, 3, 4, 6, 7, 9, 10, 12, 13, 15, 16 or 18), and a sequence having known catalytic sequence responsible for mRNA cleavage (see U.S. Pat. No. 5,093,246 or Haselhoff and Gerlach, (1988) Nature 334:585-591). For example, a derivative of a Tetrahymena L-19 IVS RNA can be constructed in which the nucleotide sequence of the active site is complementary to the nucleotide sequence to be cleaved in a 38650, 28472, 5495, 65507, 81588 or 14354-encoding mRNA. See, e.g., Cech et al. U.S. Pat. No. 4,987,071; and Cech et al. U.S. Pat. No. 5,116,742. Alternatively, 38650, 28472, 5495, 65507, 81588 or 14354 mRNA can be used to select a catalytic RNA having a specific ribonuclease activity from a pool of RNA molecules. See, e.g., Bartel, D. and Szostak, J. W. (1993) Science 261:1411-1418.

38650, 28472, 5495, 65507, 81588 or 14354 gene expression can be inhibited by targeting nucleotide sequences complementary to the regulatory region of the 38650, 28472, 5495, 65507, 81588 or 14354 (e.g., the 38650, 28472, 5495, 65507, 81588 or 14354 promoter and/or enhancers) to form triple helical structures that prevent transcription of the 38650, 28472, 5495, 65507, 81588 or 14354 gene in target cells. See generally, Helene, C., (1991) Anticancer Drug Des. 6(6):569-84; Helene, C. et al., (1992) Ann. N.Y. Acad. Sci. 660:27-36; and Maher, L. J., (1992) Bioassays 14(12):807-15. The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A 38650, 28472, 5495, 65507, 81588 or 14354 nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al., (1996) Bioorganic & Medicinal Chemistry 4 (1): 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al., (1996) supra; Perry-O'Keefe et al., Proc. Natl. Acad. Sci. 93: 14670-675.

PNAs of 38650, 28472, 5495, 65507, 81588 or 14354 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 38650, 28472, 5495, 65507, 81588 or 14354 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B., (1996) supra)); or as probes or primers for DNA sequencing or hybridization (Hyrup B. et al., (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al., (1989) Proc. Natl. Acad. Sci. USA 86:6553-6556; Lemaitre et al., (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al., (1988) Bio-Techniques 6:958-976) or intercalating agents. (See, e.g., Zon, (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a 38650, 28472, 5495, 65507, 81588 or 14354 nucleic acid of the invention, two complementary regions one having a fluorophore and one a quencher such that the molecular beacon is useful for quantitating the presence of the 38650, 28472, 5495, 65507, 81588 or 14354 nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. 5,876,930.

Isolated 38650, 28472, 5495, 65507, 81588 or 14354 Polypeptides

In another aspect, the invention features, an isolated 38650, 28472, 5495, 65507, 81588 or 14354 protein, or fragment, e.g., a biologically active portion, for use as immunogens or antigens to raise or test (or more generally to bind) anti-38650, 28472, 5495, 65507, 81588 or 14354 antibodies. 38650, 28472, 5495, 65507, 81588 or 14354 protein can be isolated from cells or tissue sources using standard protein purification techniques. 38650, 28472, 5495, 65507, 81588 or 14354 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and postranslational events. The polypeptide can be expressed in systems, e.g., cultured cells, which result in substantially the same postranslational modifications present when expressed the polypeptide is expressed in a native cell, or in systems which result in the alteration or omission of postranslational modifications, e.g., gylcosylation or cleavage, present when expressed in a native cell.

In a preferred embodiment, a 38650 polypeptide has one or more of the following characteristics:

(i) activation of an adenosine deaminase activity;

(ii) catabolism of purines;

(iii) cellular regulation of nucleic acids;

(iv) modulation of cell death;

(v) it has a molecular weight, e.g., a deduced molecular weight, amino acid composition or other physical characteristic of the polypeptide of SEQ ID NO:2;

(vi) it has an overall sequence similarity of at least 50%, preferably at least 60%, more preferably at least 70, 80, 90, or 95%, with a polypeptide of SEQ ID NO:2;

(vii) it has an adenosine deaminase family domain which preferably has an overall sequence similarity of about 70%, 80%, 90% or 95% with amino acid residues 9-344 of SEQ ID NO:2;

(viii) it has at least 70%, preferably 80%, and most preferably 95% of the cysteines found in the amino acid sequence of the native protein.

In preferred embodiments, 28472 polypeptides have one or more of the following characteristics:

(i) activation of a glycoprotease activity;

(ii) cleavage of sialoglycoproteins;

(iii) modulation of platelet function;

(iv) modulation of platelet aggregation;

(v) modulation of salt sensitivity

(vi) it has a molecular weight, e.g., a deduced molecular weight, amino acid composition or other physical characteristic of the polypeptide of SEQ ID NO:5;

(vii) it has an overall sequence similarity of at least 50%, preferably at least 60%, more preferably at least 70, 80, 90, or 95%, with a polypeptide of SEQ ID NO:5;

(viii) it has a glycoprotease family domain which preferably has an overall sequence similarity of about 70%, 80%, 90% or 95% with amino acid residues 37-372 of SEQ ID NO:5;

(ix) it has at least 70%, preferably 80%, and most preferably 95% of the cysteines found in the amino acid sequence of the native protein.

In a preferred embodiment, a 5495, 65507, 81588, or 14354 polypeptide has one or more of the following characteristics:

(i) it has the ability to regulate, sense and/or transmit an extracellular signal into a cell;

(ii) it has the ability to interact with (e.g., bind to) an extracellular signal or a cell surface receptor;

(iii) it has the ability to mobilize an intracellular molecule that participates in a signal transduction pathway (e.g., adenylate cyclase or phosphatidylinositol 4,5-bisphosphate (PIP₂), inositol 1,4,5-triphosphate (IP₃));

(iv) it has the ability to regulate polarization of the plasma membrane;

(v) it has the ability to modulate cell proliferation, cell migration, differentiation and/or cell survival;

(vi) it has the ability to modulate function, survival, morphology, proliferation and/or differentiation of cells of tissues in which 5495, 65507, 81588, or 14354 molecules are expressed;

(vii) it has a molecular weight (e.g., deduced molecular weight), amino acid composition or other physical characteristic of a 5495 protein of SEQ ID NO:8 or 65507 protein of SEQ ID NO:11 or 81588 protein of SEQ ID NO:14 or 14354 protein of SEQ ID NO:17;

(viii) it has an overall sequence similarity (identity) of at least 60%, preferably at least 70%, more preferably at least 75, 80, 85, 86, 87, 88, 89, 90, 91, 92, 93, 94, 95, 96, 97, 98, 99% or more, with a polypeptide of SEQ ID NO:8, 11, 14 or 17;

(ix) it has an N-terminal domain which is preferably about 70%, 80%, 90%, 95%, 96%, 97%, 98%, 99% or higher, identical to a polypeptide of SEQ ID NO:8, 11, 14 or 17;

(x) it has at least one transmembrane domains which is preferably about 70%, 80%, 90%, 95% or higher, identical to a polypeptide of SEQ ID NO:8, 11, 14 or 17;

(xi) it has a C-terminal domain which is preferably about 70%, 80%, 90%, 95%, 96%, 97%, 98%, 99% or higher, identical to a polypeptide of SEQ ID NO:8, 11, 14 or 17; or

(xii) it has a seven transmembrane receptor domain which preferably has an overall sequence similarity of about 70%, 80%, 90% or 95% with amino acid residues 47-279 of SEQ ID NO:8 or amino acid residues 43-285 of SEQ ID NO:11 or amino acid residues 156-326 of SEQ ID NO:14 or amino acid residues 582-845 of SEQ ID NO:17.

In a preferred embodiment the 38650, 28472, 5495, 65507, 81588 or 14354 protein, or fragment thereof, differs from the corresponding sequence in SEQ ID NO:2, 5, 8, 11, 14 or 17. In one embodiment it differs by at least one but by less than 15, 10 or 5 amino acid residues. In another it differs from the corresponding sequence in SEQ ID NO:2, 5, 8, 11, 14 or 17 by at least one residue but less than 20%, 15%, 10% or 5% of the residues in it differ from the corresponding sequence in SEQ ID NO:2, 5, 8, 11, 14 or 17. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the adenosine deaminase, glycoprotease, or 7TM receptor domain. In another preferred embodiment one or more differences are in non-active site residues, e.g. outside of the adenosine deaminase, glycoprotease, or 7TM receptor domain.

Other embodiments include a protein that contain one or more changes in amino acid sequence, e.g., a change in an amino acid residue which is not essential for activity. Such 38650, 28472, 5495, 65507, 81588 or 14354 proteins differ in amino acid sequence from SEQ ID NO:2, 5, 8, 11, 14 or 17, yet retain biological activity.

In one embodiment, a biologically active portion of a 38650, 28472, 5495, 65507, 81588 or 14354 protein includes a adenosine deaminase, glycoprotease, or 7TM receptor domain. In another embodiment, a biologically active portion of a 38650, 28472, 5495, 65507, 81588 or 14354 protein includes a protein kinase C phosphorylation domain. Moreover, other biologically active portions, in which other regions of the protein are deleted, can be prepared by recombinant techniques and evaluated for one or more of the functional activities of a native 38650, 28472, 5495, 65507, 81588 or 14354 protein.

In a preferred embodiment, the 38650, 28472, 5495, 65507, 81588 or 14354 protein has an amino acid sequence shown in SEQ ID NO:2, 5, 8, 11, 14 or 17. In other embodiments, the 38650, 28472, 5495, 65507, 81588 or 14354 protein is substantially identical to SEQ ID NO:2, 5, 8, 11, 14 or 17. In yet another embodiment, the 38650, 28472, 5495, 65507, 81588 or 14354 protein is substantially identical to SEQ ID NO:2, 5, 8, 11, 14 or 17 and retains the functional activity of the protein of SEQ ID NO:2, 5, 8, 11, 14 or 17, as described in detail above. Accordingly, in another embodiment, the 38650, 28472, 5495, 65507, 81588 or 14354 protein is a protein which includes an amino acid sequence at least about 60%, 65%, 70%, 75%, 80%, 85%, 90%, 95%, 98% or more identical to SEQ ID NO:2, 5, 8, 11, 14 or 17.

38650 28472. 5495. 65507, 81588 or 14354 Chimeric or Fusion Proteins

In another aspect, the invention provides 38650, 28472, 5495, 65507, 81588 or 14354 chimeric or fusion proteins. As used herein, a 38650, 28472, 5495, 65507, 81588 or 14354 “chimeric protein” or “fusion protein” includes a 38650, 28472, 5495, 65507, 81588 or 14354 polypeptide linked to a non-38650, 28472, 5495, 65507, 81588 or 14354 polypeptide. A “non-38650, 28472, 5495, 65507, 81588 or 14354 polypeptide” refers to a polypeptide having an amino acid sequence corresponding to a protein which is not substantially homologous to the 38650, 28472, 5495, 65507, 81588 or 14354 protein, e.g., a protein which is different from the 38650, 28472, 5495, 65507, 81588 or 14354 protein and which is derived from the same or a different organism. The 38650, 28472, 5495, 65507, 81588 or 14354 polypeptide of the fusion protein can correspond to all or a portion e.g., a fragment described herein of a 38650, 28472, 5495, 65507, 81588 or 14354 amino acid sequence. In a preferred embodiment, a 38650, 28472, 5495, 65507, 81588 or 14354 fusion protein includes at least one (or two) biologically active portion of a 38650, 28472, 5495, 65507, 81588 or 14354 protein. The non-38650, 28472, 5495, 65507, 81588 or 14354 polypeptide can be fused to the N-terminus or C-terminus of the 38650, 28472, 5495, 65507, 81588 or 14354 polypeptide.

The fusion protein can include a moiety which has a high affinity for a ligand. For example, the fusion protein can be a GST-38650, 28472, 5495, 65507, 81588 or 14354 fusion protein in which the 38650, 28472, 5495, 65507, 81588 or 14354 sequences are fused to the C-terminus of the GST sequences. Such fusion proteins can facilitate the purification of recombinant 38650, 28472, 5495, 65507, 81588 or 14354. Alternatively, the fusion protein can be a 38650, 28472, 5495, 65507, 81588 or 14354 protein containing a heterologous signal sequence at its N-terminus. In certain host cells (e.g., mammalian host cells), expression and/or secretion of 38650, 28472, 5495, 65507, 81588 or 14354 can be increased through use of a heterologous signal sequence.

Fusion proteins can include all or a part of a serum protein, e.g., a portion of an immunoglobulin (e.g., IgG, IgA, or IgE), e.g., an Fc region and/or the hinge C1 and C2 sequences of an immunoglobulin or human serum albumin.

The 38650, 28472, 5495, 65507, 81588 or 14354 fusion proteins of the invention can be incorporated into pharmaceutical compositions and administered to a subject in vivo. The 38650, 28472, 5495, 65507, 81588 or 14354 fusion proteins can be used to affect the bioavailability of a 38650, 28472, 5495, 65507, 81588 or 14354 substrate. 38650, 28472, 5495, 65507, 81588 or 14354 fusion proteins may be useful therapeutically for the treatment of disorders caused by, for example, (i) aberrant modification or mutation of a gene encoding a 38650, 28472, 5495, 65507, 81588 or 14354 protein; (ii) mis-regulation of the 38650, 28472, 5495, 65507, 81588 or 14354 gene; and (iii) aberrant post-translational modification of a 38650, 28472, 5495, 65507, 81588 or 14354 protein.

Moreover, the 38650, 28472, 5495, 65507, 81588 or 14354-fusion proteins of the invention can be used as immunogens to produce anti-38650, 28472, 5495, 65507, 81588 or 14354 antibodies in a subject, to purify 38650, 28472, 5495, 65507, 81588 or 14354 ligands and in screening assays to identify molecules which inhibit the interaction of 38650, 28472, 5495, 65507, 81588 or 14354 with a 38650, 28472, 5495, 65507, 81588 or 14354 substrate.

Expression vectors are commercially available that already encode a fusion moiety (e.g., a GST polypeptide). A 38650, 28472, 5495, 65507, 81588 or 14354-encoding nucleic acid can be cloned into such an expression vector such that the fusion moiety is linked in-frame to the 38650, 28472, 5495, 65507, 81588 or 14354 protein.

Variants of 38650, 28472, 5495, 65507, 81588 or 14354 Proteins

In another aspect, the invention also features a variant of a 38650, 28472, 5495, 65507, 81588 or 14354 polypeptide, e.g., which functions as an agonist (mimetics) or as an antagonist. Variants of the 38650, 28472, 5495, 65507, 81588 or 14354 proteins can be generated by mutagenesis, e.g., discrete point mutation, the insertion or deletion of sequences or the truncation of a 38650, 28472, 5495, 65507, 81588 or 14354 protein. An agonist of the 38650, 28472, 5495, 65507, 81588 or 14354 proteins can retain substantially the same, or a subset, of the biological activities of the naturally occurring form of a 38650, 28472, 5495, 65507, 81588 or 14354 protein. An antagonist of a 38650, 28472, 5495, 65507, 81588 or 14354 protein can inhibit one or more of the activities of the naturally occurring form of the 38650, 28472, 5495, 65507, 81588 or 14354 protein by, for example, competitively modulating a 38650, 28472, 5495, 65507, 81588 or 14354-mediated activity of a 38650, 28472, 5495, 65507, 81588 or 14354 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 38650, 28472, 5495, 65507, 81588 or 14354 protein.

Variants of a 38650, 28472, 5495, 65507, 81588 or 14354 protein can be identified by screening combinatorial libraries of mutants, e.g., truncation mutants, of a 38650, 28472, 5495, 65507, 81588 or 14354 protein for agonist or antagonist activity.

Libraries of fragments e.g., N terminal, C terminal, or internal fragments, of a 38650, 28472, 5495, 65507, 81588 or 14354 protein coding sequence can be used to generate a variegated population of fragments for screening and subsequent selection of variants of a 38650, 28472, 5495, 65507, 81588 or 14354 protein.

Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property. Recursive ensemble mutagenesis (REM), a new technique which enhances the frequency of functional mutants in the libraries, can be used in combination with the screening assays to identify 38650, 28472, 5495, 65507, 81588 or 14354 variants (Arkin and Yourvan, (1992) Proc. Natl. Acad. Sci. USA 89:7811-7815; Delgrave et al., (1993) Protein Engineering 6(3):327-331).

Cell based assays can be exploited to analyze a variegated 38650, 28472, 5495, 65507, 81588 or 14354 library. For example, a library of expression vectors can be transfected into a cell line, e.g., a cell line, which ordinarily responds to 38650, 28472, 5495, 65507, 81588 or 14354 in a substrate-dependent manner. The transfected cells are then contacted with 38650, 28472, 5495, 65507, 81588 or 14354 and the effect of the expression of the mutant on signaling by the 38650, 28472, 5495, 65507, 81588 or 14354 substrate can be detected, e.g., by measuring adenosine deaminase, glycoprotease, or 7TM receptor activity. Plasmid DNA can then be recovered from the cells which score for inhibition, or alternatively, potentiation of signaling by the 38650, 28472, 5495, 65507, 81588 or 14354 substrate, and the individual clones further characterized.

In another aspect, the invention features a method of making a 38650, 28472, 5495, 65507, 81588 or 14354 polypeptide, e.g., a peptide having a non-wild type activity, e.g., an antagonist, agonist, or super agonist of a naturally occurring 38650, 28472, 5495, 65507, 81588 or 14354 polypeptide, e.g., a naturally occurring 38650, 28472, 5495, 65507, 81588 or 14354 polypeptide. The method includes: altering the sequence of a 38650, 28472, 5495, 65507, 81588 or 14354 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a 38650, 28472, 5495, 65507, 81588 or 14354 polypeptide a biological activity of a naturally occurring 38650, 28472, 5495, 65507, 81588 or 14354 polypeptide. The method includes: altering the sequence, e.g., by substitution or deletion of one or more residues, of a 38650, 28472, 5495, 65507, 81588 or 14354 polypeptide, e.g., altering the sequence of a non-conserved region, or a domain or residue described herein, and testing the altered polypeptide for the desired activity.

Anti-38650, 28472, 5495, 65507. 81588 or 14354 Antibodies

In another aspect, the invention provides an anti-38650, 28472, 5495, 65507, 81588 or 14354 antibody. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin.

The antibody can be a polyclonal, monoclonal, recombinant, e.g., a chimeric or humanized, fully human, non-human, e.g., murine, or single chain antibody. In a preferred embodiment it has effector function and can fix complement. The antibody can be coupled to a toxin or imaging agent.

A full-length 38650, 28472, 5495, 65507, 81588 or 14354 protein or, antigenic peptide fragment of 38650, 28472, 5495, 65507, 81588 or 14354 can be used as an immunogen or can be used to identify anti-38650, 28472, 5495, 65507, 81588 or 14354 antibodies made with other immunogens, e.g., cells, membrane preparations, and the like. The antigenic peptide of 38650, 28472, 5495, 65507, 81588 or 14354 should include at least 8 amino acid residues of the amino acid sequence shown in SEQ ID NO:2, 5, 8, 11, 14 or 17 and encompasses an epitope of 38650, 28472, 5495, 65507, 81588 or 14354. Preferably, the antigenic peptide includes at least 10 amino acid residues, more preferably at least 15 amino acid residues, even more preferably at least 20 amino acid residues, and most preferably at least 30 amino acid residues.

Fragments of 38650, 28472, 5495, 65507, 81588 or 14354 can be used as immunogens or used to characterize the specificity of an antibody or antibodies against what are believed to be hydrophilic regions of the 38650, 28472, 5495, 65507, 81588 or 14354 protein. Similarly, a fragment of 38650, 28472, 5495, 65507, 81588 or 14354 can be used to make an antibody against what is believed to be a hydrophobic region of the 38650, 28472, 5495, 65507, 81588 or 14354 protein; a fragment of 38650, 28472, 5495, 65507, 81588 or 14354 can be used to make an antibody against the adenosine deaminase, glycoprotease, or 7TM receptor region of the 38650, 28472, 5495, 65507, 81588 or 14354 protein. Hydophobicity and hydrophilicity can be determined by using a Kyte-Dolittle plot as described herein.

Antibodies reactive with, or specific for, any of these regions, or other regions or domains described herein are provided.

In a preferred embodiment the antibody fails to bind an Fc receptor, e.g. it is a type which does not support Fc receptor binding or has been modified, e.g., by deletion or other mutation, such that is does not have a functional Fc receptor binding region.

Preferred epitopes encompassed by the antigenic peptide are regions of 38650, 28472, 5495, 65507, 81588 or 14354 are located on the surface of the protein, e.g., hydrophilic regions, as well as regions with high antigenicity. For example, an Emini surface probability analysis of the human 38650, 28472, 5495, 65507, 81588 or 14354 protein sequence can be used to indicate the regions that have a particularly high probability of being localized to the surface of the 38650, 28472, 5495, 65507, 81588 or 14354 protein and are thus likely to constitute surface residues useful for targeting antibody production.

In a preferred embodiment the antibody binds an epitope on any domain or region on 38650, 28472, 5495, 65507, 81588 or 14354 proteins described herein.

Chimeric, humanized, but most preferably, completely human antibodies are desirable for applications which include repeated administration, e.g., therapeutic treatment (and some diagnostic applications) of human patients.

Additionally, chimeric, humanized, and completely human antibodies are also within the scope of the invention. Chimeric, humanized, but most preferably, completely human antibodies are desirable for applications which include repeated administration, e.g., therapeutic treatment of human patients, and some diagnostic applications.

Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

The anti-38650, 28472, 5495, 65507, 81588 or 14354 antibody can be a single chain antibody. A single-chain antibody (scFV) can be engineered (see, for example, Colcher, D. et al. (1999) Ann. N Y Acad. Sci. 880:263-80; and Reiter, Y. (1996) Clin. Cancer Res. 2:245-52). The single chain antibody can be dimerized or multimerized to generate multivalent antibodies having specificities for different epitopes of the same target 38650, 28472, 5495, 65507, 81588 or 14354 protein.

In a preferred embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region. An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

An anti-38650, 28472, 5495, 65507, 81588 or 14354 antibody (e.g., monoclonal antibody) can be used to isolate 38650, 28472, 5495, 65507, 81588 or 14354 by standard techniques, such as affinity chromatography or immunoprecipitation. Moreover, an anti-38650, 28472, 5495, 65507, 81588 or 14354 antibody can be used to detect 38650, 28472, 5495, 65507, 81588 or 14354 protein (e.g., in a cellular lysate or cell supernatant) in order to evaluate the abundance and pattern of expression of the protein. Anti-38650, 28472, 5495, 65507, 81588 or 14354 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In preferred embodiments, an antibody can be made by immunizing with a purified 38650, 28472, 5495, 65507, 81588 or 14354 antigen, or a fragment thereof, e.g. a fragment described herein, a membrane associated antigen, tissues, e.g., crude tissue preparations, whole cells, preferably living cells, lysed cells, or cell fractions, e.g., membrane fractions.

Antibodies which bind only a native 38650, 28472, 5495, 65507, 81588 or 14354 protein, only denatured or otherwise non-native 38650, 28472, 5495, 65507, 81588 or 14354 protein, or which bind both, are within the invention. Antibodies with linear or conformational epitopes are within the invention. Conformational epitopes sometimes can be identified by identifying antibodies which bind to native but not denatured 38650, 28472, 5495, 65507, 81588 or 14354 protein.

Recombinant Expression Vectors Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a 38650, 28472, 5495, 65507, 81588 or 14354 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., 38650, 28472, 5495, 65507, 81588 or 14354 proteins, mutant forms of 38650, 28472, 5495, 65507, 81588 or 14354 proteins, fusion proteins, and the like).

The recombinant expression vectors of the invention can be designed for expression of 38650, 28472, 5495, 65507, 81588 or 14354 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S., (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in 38650, 28472, 5495, 65507, 81588 or 14354 activity assays, (e.g., direct assays or competitive assays described in detail below), or to generate antibodies specific for 38650, 28472, 5495, 65507, 81588 or 14354 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six (6) weeks).

To maximize recombinant protein expression in E. coli is to express the protein in host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The 38650, 28472, 5495, 65507, 81588 or 14354 expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al., (1987) Genes Dev. 1:268-277), lymphoid-specific promoters (Calame and Eaton, (1988) Adv. Immunol. 43:235-275), in particular promoters of T cell receptors (Winoto and Baltimore, (1989) EMBO J. 8:729-733) and immunoglobulins (Banerji et al., (1983) Cell 33:729-740; Queen and Baltimore, (1983) Cell 33:741-748), neuron-specific promoters (e.g., the neurofilament promoter; Byrne and Ruddle, (1989) Proc. Natl. Acad. Sci. USA 86:5473-5477), pancreas-specific promoters (Edlund et al., (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss, (1990) Science 249:374-379) and the α-fetoprotein promoter (Campes and Tilghman, (1989) Genes Dev. 3:537-546).

The invention frther provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub, H. et al., Antisense RNA as a molecular tool for genetic analysis, Reviews—Trends in Genetics, Vol. 1(1) 1986.

Another aspect the invention provides a host cell which includes a nucleic acid molecule described herein, e.g., a 38650, 28472, 5495, 65507, 81588 or 14354 nucleic acid molecule within a recombinant expression vector or a 38650, 28472, 5495, 65507, 81588 or 14354 nucleic acid molecule containing sequences which allow it to homologously recombine into a specific site of the host cell's genome. The terms “host cell” and “recombinant host cell” are used interchangeably herein. Such terms refer not only to the particular subject cell but rather also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 38650, 28472, 5495, 65507, 81588 or 14354 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation

A host cell of the invention can be used to produce (i.e., express) a 38650, 28472, 5495, 65507, 81588 or 14354 protein. Accordingly, the invention further provides methods for producing a 38650, 28472, 5495, 65507, 81588 or 14354 protein using the host cells of the invention. In one embodiment, the method includes culturing the host cell of the invention (into which a recombinant expression vector encoding a 38650, 28472, 5495, 65507, 81588 or 14354 protein has been introduced) in a suitable medium such that a 38650, 28472, 5495, 65507, 81588 or 14354 protein is produced. In another embodiment, the method further includes isolating a 38650, 28472, 5495, 65507, 81588 or 14354 protein from the medium or the host cell.

In another aspect, the invention features, a cell or purified preparation of cells which include a 38650, 28472, 5495, 65507, 81588 or 14354 transgene, or which otherwise misexpress 38650, 28472, 5495, 65507, 81588 or 14354. The cell preparation can consist of human or non-human cells, e.g., rodent cells, e.g., mouse or rat cells, rabbit cells, or pig cells. In preferred embodiments, the cell or cells include a 38650, 28472, 5495, 65507, 81588 or 14354 transgene, e.g., a heterologous form of a 38650, 28472, 5495, 65507, 81588 or 14354, e.g., a gene derived from humans (in the case of a non-human cell). The 38650, 28472, 5495, 65507, 81588 or 14354 transgene can be misexpressed, e.g., overexpressed or underexpressed. In other preferred embodiments, the cell or cells include a gene which misexpress an endogenous 38650, 28472, 5495, 65507, 81588 or 14354, e.g., a gene the expression of which is disrupted, e.g., a knockout. Such cells can serve as a model for studying disorders which are related to mutated or mis-expressed 38650, 28472, 5495, 65507, 81588 or 14354 alleles or for use in drug screening.

In another aspect, the invention features, a human cell, e.g., a hematopoietic stem cell, transformed with nucleic acid which encodes a subject 38650, 28472, 5495, 65507, 81588 or 14354 polypeptide.

Also provided are cells or a purified preparation thereof, e.g., human cells, in which an endogenous 38650, 28472, 5495, 65507, 81588 or 14354 is under the control of a regulatory sequence that does not normally control the expression of the endogenous 38650, 28472, 5495, 65507, 81588 or 14354 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 38650, 28472, 5495, 65507, 81588 or 14354 gene. For example, an endogenous 38650, 28472, 5495, 65507, 81588 or 14354 gene, e.g., a gene which is “ranscriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

Transgenic Animals

The invention provides non-human transgenic animals. Such animals are useful for studying the function and/or activity of a 38650, 28472, 5495, 65507, 81588 or 14354 protein and for identifying and/or evaluating modulators of 38650, 28472, 5495, 65507, 81588 or 14354 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 38650, 28472, 5495, 65507, 81588 or 14354 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a 38650, 28472, 5495, 65507, 81588 or 14354 protein to particular cells. A transgenic founder animal can be identified based upon the presence of a 38650, 28472, 5495, 65507, 81588 or 14354 transgene in its genome and/or expression of 38650, 28472, 5495, 65507, 81588 or 14354 mRNA in tissues or cells of the animals. A transgenic founder animal can then be used to breed additional animals carrying the transgene. Moreover, transgenic animals carrying a transgene encoding a 38650, 28472, 5495, 65507, 81588 or 14354 protein can further be bred to other transgenic animals carrying other transgenes.

03101 38650,28472, 5495, 65507, 81588 or 14354 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed herein.

Uses

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a 38650, 28472, 5495, 65507, 81588 or 14354 protein (e.g., via a recombinant expression vector in a host cell in gene therapy applications), to detect a 38650, 28472, 5495, 65507, 81588 or 14354 mRNA (e.g., in a biological sample) or a genetic alteration in a 38650, 28472, 5495, 65507, 81588 or 14354 gene, and to modulate 38650, 28472, 5495, 65507, 81588 or 14354 activity, as described further below. The 38650, 28472, 5495, 65507, 81588 or 14354 proteins can be used to treat disorders characterized by insufficient or excessive production of a 38650, 28472, 5495, 65507, 81588 or 14354 substrate or production of 38650, 28472, 5495, 65507, 81588 or 14354 inhibitors. In addition, the 38650, 28472, 5495, 65507, 81588 or 14354 proteins can be used to screen for naturally occurring 38650, 28472, 5495, 65507, 81588 or 14354 substrates, to screen for drugs or compounds which modulate 38650, 28472, 5495, 65507, 81588 or 14354 activity, as well as to treat disorders characterized by insufficient or excessive production of 38650, 28472, 5495, 65507, 81588 or 14354 protein or production of 38650, 28472, 5495, 65507, 81588 or 14354 protein forms which have decreased, aberrant or unwanted activity compared to 38650, 28472, 5495, 65507, 81588 or 14354 wild-type protein. Such disorders include those characterized by aberrant signaling or aberrant, e.g., hyperproliferative, cell growth. Moreover, the anti-38650, 28472, 5495, 65507, 81588 or 14354 antibodies of the invention can be used to detect and isolate 38650, 28472, 5495, 65507, 81588 or 14354 proteins, regulate the bioavailability of 38650, 28472, 5495, 65507, 81588 or 14354 proteins, and modulate 38650, 28472, 5495, 65507, 81588 or 14354 activity.

A method of evaluating a compound for the ability to interact with, e.g., bind, a subject 38650, 28472, 5495, 65507, 81588 or 14354 polypeptide is provided. The method includes: contacting the compound with the subject 38650, 28472, 5495, 65507, 81588 or 14354 polypeptide; and evaluating ability of the compound to interact with, e.g., to bind or form a complex with the subject 38650, 28472, 5495, 65507, 81588 or 14354 polypeptide. This method can be performed in vitro, e.g., in a cell free system, or in vivo, e.g., in a two-hybrid interaction trap assay. This method can be used to identify naturally occurring molecules which interact with subject 38650, 28472, 5495, 65507, 81588 or 14354 polypeptide. It can also be used to find natural or synthetic inhibitors of subject 38650, 28472, 5495, 65507, 81588 or 14354 polypeptide. Screening methods are discussed in more detail below.

Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to 38650, 28472, 5495, 65507, 81588 or 14354 proteins, have a stimulatory or inhibitory effect on, for example, 38650, 28472, 5495, 65507, 81588 or 14354 expression or 38650, 28472, 5495, 65507, 81588 or 14354 activity, or have a stimulatory or inhibitory effect on, for example, the expression or activity of a 38650, 28472, 5495, 65507, 81588 or 14354 substrate. Compounds thus identified can be used to modulate the activity of target gene products (e.g., 38650, 28472, 5495, 65507, 81588 or 14354 genes) in a therapeutic protocol, to elaborate the biological function of the target gene product, or to identify compounds that disrupt normal target gene interactions.

In one embodiment, the invention provides assays for screening candidate or test compounds which are substrates of a 38650, 28472, 5495, 65507, 81588 or 14354 protein or polypeptide or a biologically active portion thereof. In another embodiment, the invention provides assays for screening candidate or test compounds which bind to or modulate the activity of a 38650, 28472, 5495, 65507, 81588 or 14354 protein or polypeptide or a biologically active portion thereof.

The test compounds of the present invention can be obtained using any of the numerous approaches in combinatorial library methods known in the art, including: biological libraries; peptoid libraries [libraries of molecules having the finctionalities of peptides, but with a novel, non-peptide backbone which are resistant to enzymatic degradation but which nevertheless remain bioactive] (see, e.g., Zuckermann, R. N. et al., J. Med. Chem. 1994, 37: 2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al., (1994) Proc. Natl. Acad. Sci. USA 91:11422; Zuckermann et al., (1994). J. Med. Chem. 37:2678; Cho et al., (1993) Science 261:1303; Carrell et al., (1994)Angew. Chem. Int. Ed. Engl. 33:2059; Carell et al., (1994) Angew. Chem. Int. Ed. Engl. 33:2061; and in Gallop et al., (1994) J. Med. Chem. 37:1233.

Libraries of compounds may be presented in solution (e.g., Houghten, (1992) Biotechniques 13:412-421), or on beads (Lam, (1991) Nature 354:82-84), chips (Fodor, (1993) Nature 364:555-556), bacteria or spores (Ladner, U.S. Pat. No. 5,223,409), plasmids (Cull et al., (1992) Proc. Natl. Acad. Sci. USA 89:1865-1869) or on phage (Scott and Smith, (1990) Science 249:386-390); (Devlin, (1990) Science 249:404-406); (Cwirla et al., (1990) Proc. Natl. Acad. Sci. 87:6378-6382); (Felici, (1991) J. Mol. Biol. 222:301-310); (Ladnersupra.).

In one embodiment, an assay is a cell-based assay in which a cell which expresses a 38650, 28472, 5495, 65507, 81588 or 14354 protein or biologically active portion thereof is contacted with a test compound, and the ability of the test compound to modulate 38650, 28472, 5495, 65507, 81588 or 14354 activity is determined. Determining the ability of the test compound to modulate 38650, 28472, 5495, 65507, 81588 or 14354 activity can be accomplished by monitoring, for example, adenosine deaminase, glycoprotease, or 7TM receptor activity. The cell, for example, can be of mammalian origin, e.g., human. Cell homogenates, or fractions, preferably membrane containing fractions, can also be tested.

The ability of the test compound to modulate 38650, 28472, 5495, 65507, 81588 or 14354 binding to a compound, e.g., a 38650, 28472, 5495, 65507, 81588 or 14354 substrate, or to bind to 38650, 28472, 5495, 65507, 81588 or 14354 can also be evaluated. This can be accomplished, for example, by coupling the compound, e.g., the substrate, with a radioisotope or enzymatic label such that binding of the compound, e.g., the substrate, to 38650, 28472, 5495, 65507, 81588 or 14354 can be determined by detecting the labeled compound, e.g., substrate, in a complex. Alternatively, 38650, 28472, 5495, 65507, 81588 or 14354 could be coupled with a radioisotope or enzymatic label to monitor the ability of a test compound to modulate 38650, 28472, 5495, 65507, 81588 or 14354 binding to a 38650, 28472, 5495, 65507, 81588 or 14354 substrate in a complex. For example, compounds (e.g., 38650, 28472, 5495, 65507, 81588 or 14354 substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 38650, 28472, 5495, 65507, 81588 or 14354 substrate) to interact with 38650, 28472, 5495, 65507, 81588 or 14354 with or without the labeling of any of the interactants can be evaluated. For example, a microphysiometer can be used to detect the interaction of a compound with 38650, 28472, 5495, 65507, 81588 or 14354 without the labeling of either the compound or the 38650, 28472, 5495, 65507, 81588 or 14354. McConnell, H. M. et al., (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 38650, 28472, 5495, 65507, 81588 or 14354.

In yet another embodiment, a cell-free assay is provided in which a 38650, 28472, 5495, 65507, 81588 or 14354 protein or biologically active portion thereof is contacted with a test compound and the ability of the test compound to bind to the 38650, 28472, 5495, 65507, 81588 or 14354 protein or biologically active portion thereof is evaluated. Preferred biologically active portions of the 38650, 28472, 5495, 65507, 81588 or 14354 proteins to be used in assays of the present invention include fragments which participate in interactions with non-38650, 28472, 5495, 65507, 81588 or 14354 molecules, e.g., fragments with high surface probability scores.

Soluble and/or membrane-bound forms of isolated proteins (e.g., 38650, 28472, 5495, 65507, 81588 or 14354 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

In one embodiment, assays are performed where the ability of an agent to block adenosine deaminase, glycoprotease, or 7TM receptor activity within a cell is evaluated.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of typtophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 38650, 28472, 5495, 65507, 81588 or 14354 protein to bind to a target molecule can be accomplished using real-time Biomolecular Interaction Analysis (BIA) (see, e.g., Sjolander, S. and Urbaniczky, C., (1991) Anal. Chem. 63:2338-2345 and Szabo et al., (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either 38650, 28472, 5495, 65507, 81588 or 14354, an anti-38650, 28472, 5495, 65507, 81588 or 14354 antibody or its target molecule to facilitate separation of complexed from uncomplexed forms of one or both of the proteins, as well as to accommodate automation of the assay. Binding of a test compound to a 38650, 28472, 5495, 65507, 81588 or 14354 protein, or interaction of a 38650, 28472, 5495, 65507, 81588 or 14354 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/38650, 28472, 5495, 65507, 81588 or 14354 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or 38650, 28472, 5495, 65507, 81588 or 14354 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 38650, 28472, 5495, 65507, 81588 or 14354 binding or activity determined using standard techniques.

Other techniques for immobilizing either a 38650, 28472, 5495, 65507, 81588 or 14354 protein or a target molecule on matrices include using conjugation of biotin and streptavidin. Biotinylated 38650, 28472, 5495, 65507, 81588 or 14354 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with 38650, 28472, 5495, 65507, 81588 or 14354 protein or target molecules but which do not interfere with binding of the 38650, 28472, 5495, 65507, 81588 or 14354 protein to its target molecule. Such antibodies can be derivatized to the wells of the plate, and unbound target or 38650, 28472, 5495, 65507, 81588 or 14354 protein trapped in the wells by antibody conjugation. Methods for detecting such complexes, in addition to those described above for the GST-immobilized complexes, include immunodetection of complexes using antibodies reactive with the 38650, 28472, 5495, 65507, 81588 or 14354 protein or target molecule, as well as enzyme-linked assays which rely on detecting an enzymatic activity associated with the 38650, 28472, 5495, 65507, 81588 or 14354 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci 1993 Aug;18(8):284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. Current Protocols in Molecular Biology 1999, J. Wiley: New York). Such resins and chromatographic techniques are known to one skilled in the art (see, e.g., Heegaard, N. H., J. Mol. Recognit. 1998 Winter;11(1-6):141-8; Hage, D. S., and Tweed, S. A., J. Chromatogr. B Biomed. Sci. Appl. Oct. 10, 1997;699(1-2):499-525). Further, fluorescence energy transfer may also be conveniently utilized, as described herein, to detect binding without further purification of the complex from solution.

In a preferred embodiment, the assay includes contacting the 38650, 28472, 5495, 65507, 81588 or 14354 protein or biologically active portion thereof with a known compound which binds 38650, 28472, 5495, 65507, 81588 or 14354 to form an assay mixture, contacting the assay mixture with a test compound, and determining the ability of the test compound to interact with a 38650, 28472, 5495, 65507, 81588 or 14354 protein, wherein determining the ability of the test compound to interact with a 38650, 28472, 5495, 65507, 81588 or 14354 protein includes determining the ability of the test compound to preferentially bind to 38650, 28472, 5495, 65507, 81588 or 14354 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the 38650, 28472, 5495, 65507, 81588 or 14354 genes herein identified. In an alternative embodiment, the invention provides methods for determining the ability of the test compound to modulate the activity of a 38650, 28472, 5495, 65507, 81588 or 14354 protein through modulation of the activity of a downstream effector of a 38650, 28472, 5495, 65507, 81588 or 14354 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), e.g., a substrate, a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the 38650, 28472, 5495, 65507, 81588 or 14354 proteins can be used as “bait proteins” in a two-hybrid assay or three-hybrid assay (see, e.g., U.S. Pat. No. 5,283,317; Zervos et al., (1993) Cell 72:223-232; Madura et al., (1993) J. Biol. Chem. 268:12046-12054; Bartel et al., (1993) Biotechniques 14:920-924; Iwabuchi et al., (1993) Oncogene 8:1693-1696; and Brent W094/10300), to identify other proteins, which bind to or interact with 38650, 28472, 5495, 65507, 81588 or 14354 (“38650, 28472, 5495, 65507, 81588 or 14354-binding proteins” or “38650, 28472, 5495, 65507, 81588 or 14354-bp”) and are involved in 38650, 28472, 5495, 65507, 81588 or 14354 activity. Such 38650, 28472, 5495, 65507, 81588 or 14354-bps can be activators or inhibitors of signals by the 38650, 28472, 5495, 65507, 81588 or 14354 proteins or 38650, 28472, 5495, 65507, 81588 or 14354 targets as, for example, downstream elements of a 38650, 28472, 5495, 65507, 81588 or 14354-mediated signaling pathway.

The two-hybrid system is based on the modular nature of most transcription factors, which consist of separable DNA-binding and activation domains. Briefly, the assay utilizes two different DNA constructs. In one construct, the gene that codes for a 38650, 28472, 5495, 65507, 81588 or 14354 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: 38650, 28472, 5495, 65507, 81588 or 14354 protein can be the fused to the activator domain.) If the “bait” and the “prey” proteins are able to interact, in vivo, forming a 38650, 28472, 5495, 65507, 81588 or 14354-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 38650, 28472, 5495, 65507, 81588 or 14354 protein.

In another embodiment, modulators of 38650, 28472, 5495, 65507, 81588 or 14354 expression are identified. For example, a cell or cell free mixture is contacted with a candidate compound and the expression of 38650, 28472, 5495, 65507, 81588 or 14354 mRNA or protein evaluated relative to the level of expression of 38650, 28472, 5495, 65507, 81588 or 14354 mRNA or protein in the absence of the candidate compound. When expression of 38650, 28472, 5495, 65507, 81588 or 14354 mRNA or protein is greater in the presence of the candidate compound than in its absence, the candidate compound is identified as a stimulator of 38650, 28472, 5495, 65507, 81588 or 14354 mRNA or protein expression. Alternatively, when expression of 38650, 28472, 5495, 65507, 81588 or 14354 mRNA or protein is less (statistically significantly less) in the presence of the candidate compound than in its absence, the candidate compound is identified as an inhibitor of 38650, 28472, 5495, 65507, 81588 or 14354 mRNA or protein expression. The level of 38650, 28472, 5495, 65507, 81588 or 14354 mRNA or protein expression can be determined by methods described herein for detecting 38650, 28472, 5495, 65507, 81588 or 14354 mRNA or protein.

In another aspect, the invention pertains to a combination of two or more of the assays described herein. For example, a modulating agent can be identified using a cell-based or a cell free assay, and the ability of the agent to modulate the activity of a 38650, 28472, 5495, 65507, 81588 or 14354 protein can be confirmed in vivo, e.g., in an animal.

This invention further pertains to novel agents identified by the above-described screening assays. Accordingly, it is within the scope of this invention to further use an agent identified as described herein (e.g., a 38650, 28472, 5495, 65507, 81588 or 14354 modulating agent, an antisense 38650, 28472, 5495, 65507, 81588 or 14354 nucleic acid molecule, a 38650, 28472, 5495, 65507, 81588 or 14354-specific antibody, or a 38650, 28472, 5495, 65507, 81588 or 14354-binding partner) in an appropriate animal model to determine the efficacy, toxicity, side effects, or mechanism of action, of treatment with such an agent. Furthermore, novel agents identified by the above-described screening assays can be used for treatments as described herein.

Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate 38650, 28472, 5495, 65507, 81588 or 14354 with a disease; (ii) identify an individual from a minute biological sample (tissue typing); and (iii) aid in forensic identification of a biological sample. These applications are described in the subsections below.

Chromosome Mapping

The 38650, 28472, 5495, 65507, 81588 or 14354 nucleotide sequences or portions thereof can be used to map the location of the 38650, 28472, 5495, 65507, 81588 or 14354 genes on a chromosome. This process is called chromosome mapping. Chromosome mapping is useful in correlating the 38650, 28472, 5495, 65507, 81588 or 14354 sequences with genes associated with disease.

Briefly, 38650, 28472, 5495, 65507, 81588 or 14354 genes can be mapped to chromosomes by preparing PCR primers (preferably 15-25 bp in length) from the 38650, 28472, 5495, 65507, 81588 or 14354 nucleotide sequences. These primers can then be used for PCR screening of somatic cell hybrids containing individual human chromosomes. Only those hybrids containing the human gene corresponding to the 38650, 28472, 5495, 65507, 81588 or 14354 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio P. et al., (1983) Science 220:919-924).

Other mapping strategies e.g., in situ hybridization (described in Fan, Y. et al., (1990) Proc. Natl. Acad. Sci. USA, 87:6223-27), pre-screening with labeled flow-sorted chromosomes, and pre-selection by hybridization to chromosome specific cDNA libraries can be used to map 38650, 28472, 5495, 65507, 81588 or 14354 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al., (1987) Nature, 325:783-787.

Moreover, differences in the DNA sequences between individuals affected and unaffected with a disease associated with the 38650, 28472, 5495, 65507, 81588 or 14354 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

38650, 28472, 5495, 65507, 81588 or 14354 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 38650, 28472, 5495, 65507, 81588 or 14354 nucleotide sequences described herein can be used to prepare two PCR primers from the 5′ and 3+ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, 4, 7, 10, 13 or 16 can provide positive individual identification with a panel of perhaps 10 to 1,000 primers which each yield a noncoding amplified sequence of 100 bases. If predicted coding sequences, such as those in SEQ ID NO:3, 6, 9, 12, 15, or 18 are used, a more appropriate number of primers for positive individual identification would be 500-2,000.

If a panel of reagents from 38650, 28472, 5495, 65507, 81588 or 14354 nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial 38650, 28472, 5495, 65507, 81588 or 14354 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1, 4, 7, 10, 13 or 16 (e.g., fragments derived from the noncoding regions of SEQ ID NO:1, 4, 7, 10, 13 or 16 having a length of at least 20 bases, preferably at least 30 bases) are particularly appropriate for this use.

The 38650, 28472, 5495, 65507, 81588 or 14354 nucleotide sequences described herein can further be used to provide polynucleotide reagents, e.g., labeled or labelable probes which can be used in, for example, an in situ hybridization technique, to identify a specific tissue, e.g., a tissue containing adenosine deaminase, glycoprotease, or 7TM receptor activity. This can be very useful in cases where a forensic pathologist is presented with a tissue of unknown origin. Panels of such 38650, 28472, 5495, 65507, 81588 or 14354 probes can be used to identify tissue by species and/or by organ type.

In a similar fashion, these reagents, e.g., 38650, 28472, 5495, 65507, 81588 or 14354 primers or probes can be used to screen tissue culture for contamination (i.e. screen for the presence of a mixture of different types of cells in a culture).

Predictive Medicine

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes 38650, 28472, 5495, 65507, 81588 or 14354.

Such disorders include, e.g., a disorder associated with the misexpression of 38650, 28472, 5495, 65507, 81588 or 14354, or adenosine deaminase, glycoprotease, or seven transmembrane receptor related disorder.

The method includes one or more of the following: detecting, in a tissue of the subject, the presence or absence of a mutation which affects the expression of the 38650, 28472, 5495, 65507, 81588 or 14354 gene, or detecting the presence or absence of a mutation in a region which controls the expression of the gene, e.g., a mutation in the 5′ control region; detecting, in a tissue of the subject, the presence or absence of a mutation which alters the structure of the 38650, 28472, 5495, 65507, 81588 or 14354 gene; detecting, in atissue of the subject, the misexpression of the 38650, 28472, 5495, 65507, 81588 or 14354 gene, at the mRNA level, e.g., detecting a non-wild type level of a mRNA; detecting, in a tissue of the subject, the misexpression of the gene, at the protein level, e.g., detecting a non-wild type level of a 38650, 28472, 5495, 65507, 81588 or 14354 polypeptide.

In preferred embodiments the method includes: ascertaining the existence of at least one of: a deletion of one or more nucleotides from the 38650, 28472, 5495, 65507, 81588 or 14354 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO:1 naturally occurring mutants thereof or 5′ or 3′ flanking sequences naturally associated with the 38650, 28472, 5495, 65507, 81588 or 14354 gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the 38650, 28472, 5495, 65507, 81588 or 14354 gene; the presence of a non-wild type splicing pattern of a messenger RNA transcript of the gene; or a non-wild type level of 38650, 28472, 5495, 65507, 81588 or 14354.

Methods of the invention can be used prenatally or to determine if a subject's offspring will be at risk for a disorder.

In preferred embodiments the method includes determining the structure of a 38650, 28472, 5495, 65507, 81588 or 14354 gene, an abnormal structure being indicative of risk for the disorder.

In preferred embodiments the method includes contacting a sample form the subject with an antibody to the 38650, 28472, 5495, 65507, 81588 or 14354 protein or a nucleic acid, which hybridizes specifically with the gene. These and other embodiments are discussed below.

Diagnostic and Prognostic Assays

The presence, level, or absence of 38650, 28472, 5495, 65507, 81588 or 14354 protein or nucleic acid in a biological sample can be evaluated by obtaining a biological sample from a test subject and contacting the biological sample with a compound or an agent capable of detecting 38650, 28472, 5495, 65507, 81588 or 14354 protein or nucleic acid (e.g., mRNA, genomic DNA) that encodes 38650, 28472, 5495, 65507, 81588 or 14354 protein such that the presence of 38650, 28472, 5495, 65507, 81588 or 14354 protein or nucleic acid is detected in the biological sample. The term “biological sample” includes tissues, cells and biological fluids isolated from a subject, as well as tissues, cells and fluids present within a subject. A preferred biological sample is serum. The level of expression of the 38650, 28472, 5495, 65507, 81588 or 14354 gene can be measured in a number of ways, including, but not limited to: measuring the mRNA encoded by the 38650, 28472, 5495, 65507, 81588 or 14354 genes; measuring the amount of protein encoded by the 38650, 28472, 5495, 65507, 81588 or 14354 genes; or measuring the activity of the protein encoded by the 38650, 28472, 5495, 65507, 81588 or 14354 genes.

The level of mRNA corresponding to the 38650, 28472, 5495, 65507, 81588 or 14354 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 38650, 28472, 5495, 65507, 81588 or 14354 nucleic acid, such as the nucleic acid of SEQ ID NO:1, or the DNA insert of the plasmid deposited with ATCC as Accession Number ______, or a portion thereof, such as an oligonucleotide of at least 7, 15, 30, 50, 100, 250 or 500 nucleotides in length and sufficient to specifically hybridize under stringent conditions to 38650, 28472, 5495, 65507, 81588 or 14354 mRNA or genomic DNA. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by the 38650, 28472, 5495, 65507, 81588 or 14354 genes.

The level of mRNA in a sample that is encoded by one of 38650, 28472, 5495, 65507, 81588 or 14354 can be evaluated with nucleic acid amplification, e.g., by rtPCR (Mullis, 1987, U.S. Pat. No. 4,683,202), ligase chain reaction (Barany, 1991, Proc. Natl. Acad. Sci. USA 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the 38650, 28472, 5495, 65507, 81588 or 14354 gene being analyzed.

In another embodiment, the methods further contacting a control sample with a compound or agent capable of detecting 38650, 28472, 5495, 65507, 81588 or 14354 mRNA, or genomic DNA, and comparing the presence of 38650, 28472, 5495, 65507, 81588 or 14354 mRNA or genomic DNA in the control sample with the presence of 38650, 28472, 5495, 65507, 81588 or 14354 mRNA or genomic DNA in the test sample.

A variety of methods can be used to determine the level of protein encoded by 38650, 28472, 5495, 65507, 81588 or 14354. In general, these methods include contacting an agent that selectively binds to the protein, such as an antibody with a sample, to evaluate the level of protein in the sample. In a preferred embodiment, the antibody bears. a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 38650, 28472, 5495, 65507, 81588 or 14354 protein in a biological sample in vitro as well as in vivo. In vitro techniques for detection of 38650, 28472, 5495, 65507, 81588 or 14354 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 38650, 28472, 5495, 65507, 81588 or 14354 protein include introducing into a subject a labeled anti-38650, 28472, 5495, 65507, 81588 or 14354 antibody. For example, the antibody can be labeled with a radioactive marker whose presence and location in a subject can be detected by standard imaging techniques.

In another embodiment, the methods further include contacting the control sample with a compound or agent capable of detecting 38650, 28472, 5495, 65507, 81588 or 14354 protein, and comparing the presence of 38650, 28472, 5495, 65507, 81588 or 14354 protein in the control sample with the presence of 38650, 28472, 5495, 65507, 81588 or 14354 protein in the test sample.

The invention also includes kits for detecting the presence of 38650, 28472, 5495, 65507, 81588 or 14354 in a biological sample. For example, the kit can include a compound or agent capable of detecting 38650, 28472, 5495, 65507, 81588 or 14354 protein or mRNA in a biological sample; and a standard. The compound or agent can be packaged in a suitable container. The kit can further comprise instructions for using the kit to detect 38650, 28472, 5495, 65507, 81588 or 14354 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein-stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted 38650, 28472, 5495, 65507, 81588 or 14354 expression or activity. As used herein, the term

“unwanted” includes an unwanted phenomenon involved in a biological response such as pain or deregulated cell proliferation.

In one embodiment, a disease or disorder associated with aberrant or unwanted 38650, 28472, 5495, 65507, 81588 or 14354 expression or activity is identified. A test sample is obtained from a subject and 38650, 28472, 5495, 65507, 81588 or 14354 protein or nucleic acid (e.g., mRNA or genomic DNA) is evaluated, wherein the level, e.g., the presence or absence, of 38650, 28472, 5495, 65507, 81588 or 14354 protein or nucleic acid is diagnostic for a subject having or at risk of developing a disease or disorder associated with aberrant or unwanted 38650, 28472, 5495, 65507, 81588 or 14354 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 38650, 28472, 5495, 65507, 81588 or 14354 expression or activity. For example, such methods can be used to determine whether a subject can be effectively treated with an agent for a adenosine deaminase, glycoprotease, or seven transmembrane receptor related disorder.

The methods of the invention can also be used to detect genetic alterations in a 38650, 28472, 5495, 65507, 81588 or 14354 gene, thereby determining if a subject with the altered gene is at risk for a disorder characterized by misregulation in 38650, 28472, 5495, 65507, 81588 or 14354 protein activity or nucleic acid expression, such as a adenosine deaminase, glycoprotease, or seven transmembrane receptor related disorder. In preferred embodiments, the methods include detecting, in a sample from the subject, the presence or absence of a genetic alteration characterized by at least one of an alteration affecting the integrity of a gene encoding a 38650, 28472, 5495, 65507, 81588 or 14354-protein, or the mis-expression of the 38650, 28472, 5495, 65507, 81588 or 14354 gene. For example, such genetic alterations can be detected by ascertaining the existence of at least one of 1) a deletion of one or more nucleotides from a 38650, 28472, 5495, 65507, 81588 or 14354 gene; 2) an addition of one or more nucleotides to a 38650, 28472, 5495, 65507, 81588 or 14354 gene; 3) a substitution of one or more nucleotides of a 38650, 28472, 5495, 65507, 81588 or 14354 gene, 4) a chromosomal rearrangement of a 38650, 28472, 5495, 65507, 81588 or 14354 gene; 5) an alteration in the level of a messenger RNA transcript of a 38650, 28472, 5495, 65507, 81588 or 14354 gene, 6) aberrant modification of a 38650, 28472, 5495, 65507, 81588 or 14354 gene, such as of the methylation pattern of the genomic DNA, 7) the presence of a non-wild type splicing pattern of a messenger RNA transcript of a 38650, 28472, 5495, 65507, 81588 or 14354 gene, 8) a non-wild type level of a 38650, 28472, 5495, 65507, 81588 or 14354-protein, 9) allelic loss of a 38650, 28472, 5495, 65507, 81588 or 14354 gene, and 10) inappropriate post-translational modification of a 38650, 28472, 5495, 65507, 81588 or 14354-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the 38650, 28472, 5495, 65507, 81588 or 14354-gene. This method can include the steps of collecting a sample of cells from a subject, isolating nucleic acid (e.g., genomic, mRNA or both) from the sample, contacting the nucleic acid sample with one or more primers which specifically hybridize to a 38650, 28472, 5495, 65507, 81588 or 14354 gene under conditions such that hybridization and amplification of the 38650, 28472, 5495, 65507, 81588 or 14354-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al., (1988) Bio-Technology 6:1197), or other nucleic acid amplification methods, followed by the detection of the amplified molecules using techniques known to those of skill in the art.

In another embodiment, mutations in a 38650, 28472, 5495, 65507, 81588 or 14354 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 38650, 28472, 5495, 65507, 81588 or 14354 can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. The arrays can have a high density of addresses, e.g., can contain hundreds or thousands of oligonucleotides probes (Cronin, M. T. et al., (1996) Human Mutation 7: 244-255; Kozal, M. J. et al., (1996) Nature Medicine 2:753-759). For example, genetic mutations in 38650, 28472, 5495, 65507, 81588 or 14354 can be identified in two dimensional arrays containing light-generated DNA probes as described in Cronin, M. T. et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 38650, 28472, 5495, 65507, 81588 or 14354 gene and detect mutations by comparing the sequence of the sample 38650, 28472, 5495, 65507, 81588 or 14354 with the corresponding wild-type (control) sequence. Automated sequencing procedures can be utilized when performing the diagnostic assays ((1995) Biotechniques 19:448), including sequencing by mass spectrometry.

Other methods for detecting mutations in the 38650, 28472, 5495, 65507, 81588 or 14354 gene include methods in which protection from cleavage agents is used to detect mismatched bases in RNA/RNA or RNA/DNA heteroduplexes (Myers et al., (1985) Science 230:1242; Cotton et al., (1988) Proc. Natl. Acad. Sci. USA 85:4397; Saleeba et al., (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 38650, 28472, 5495, 65507, 81588 or 14354 cDNAs obtained from samples of cells. For example, the mutY enzyme of E. coli cleaves A at G/A mismatches and the thymidine DNA glycosylase from HeLa cells cleaves T at G/T mismatches (Hsu et al., (1994) Carcinogenesis 15:1657-1662; U.S. Pat. No. 5,459,039).

In other embodiments, alterations in electrophoretic mobility will be used to identify mutations in 38650, 28472, 5495, 65507, 81588 or 14354 genes. For example, single strand conformation polymorphism (SSCP) may be used to detect differences in electrophoretic mobility between mutant and wild type nucleic acids (Orita et al., (1989) Proc. Natl. Acad. Sci. USA: 86:2766, see also Cotton, (1993) Mutat. Res. 285:125-144; and Hayashi, (1992) Genet. Anal Tech. Appl. 9:73-79). Single-stranded DNA fragments of sample and control 38650, 28472, 5495, 65507, 81588 or 14354 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al., (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al., (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner, (1987) Biophys. Chem. 265:12753).

Examples of other techniques for detecting point mutations include, but are not limited to, selective oligonucleotide hybridization, selective amplification, or selective primer extension (Saiki et al., (1986) Nature 324:163); Saiki et al., (1989) Proc. Natl. Acad. Sci. USA 86:6230).

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al., (1989) Nucleic Acids Res. 17:2437-2448) or at the extreme 3′ end of one primer where, under appropriate conditions, mismatch can prevent, or reduce polymerase extension (Prossner, (1993) Tibtech 11:238). In addition it may be desirable to introduce a novel restriction site in the region of the mutation to create cleavage-based detection (Gasparini et al., (1992) Mol. Cell Probes 6:1). It is anticipated that in certain embodiments amplification may also be performed using Taq ligase for amplification (Barany, (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g. in clinical settings to diagnose patients exhibiting symptoms or family history of a disease or illness involving a 38650, 28472, 5495, 65507, 81588 or 14354 gene.

Use of 38650, 28472, 5495, 65507, 81588 or 14354 Molecules as Surrogate Markers

The 38650, 28472, 5495, 65507, 81588 or 14354 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 38650, 28472, 5495, 65507, 81588 or 14354 molecules of the invention may be detected, and may be correlated with one or more biological states in vivo. For example, the 38650, 28472, 5495, 65507, 81588 or 14354 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 38650, 28472, 5495, 65507, 81588 or 14354 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a 38650, 28472, 5495, 65507, 81588 or 14354 marker) transcription or expression, the amplified marker may be in a quantity which is more readily detectable than the drug itself. Also, the marker may be more easily detected due to the nature of the marker itself; for example, using the methods described herein, anti-38650, 28472, 5495, 65507, 81588 or 14354 antibodies maybe employed in an immune-based detection system for a 38650, 28472, 5495, 65507, 81588 or 14354 protein marker, or 38650, 28472, 5495, 65507, 81588 or 14354-specific radiolabeled probes may be used to detect a 38650, 28472, 5495, 65507, 81588 or 14354 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Phann. 56 Suppl. 3: S16-S20.

The 38650, 28472, 5495, 65507, 81588 or 14354 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., 38650, 28472, 5495, 65507, 81588 or 14354 protein or RNA) for specific tumor markers in a subject, a drug or course of treatment may be selected that is optimized for the treatment of the specific tumor likely to be present in the subject. Similarly, the presence or absence of a specific sequence mutation in 38650, 28472, 5495, 65507, 81588 or 14354 DNA may correlate 38650, 28472, 5495, 65507, 81588 or 14354 drug response. The use of pharmacogenomic markers therefore permits the application of the most appropriate treatment for each subject without having to administer the therapy.

Pharmaceutical Compositions

The nucleic acid and polypeptides, fragments thereof, as well as anti-38650, 28472, 5495, 65507, 81588 or 14354 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifingal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., ((1997) J. Acquired Immune Deficiency Syndromes and Human Retrovirology 14:193).

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof Therapeutic agents include, but are not limited to, antimetabolites (e.g., methotrexate, 6-mercaptopurine, 6-thioguanine. cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment

The present invention provides for both prophylactic and therapeutic methods of treating a subject at risk of (or susceptible to) a disorder or having a disorder associated with aberrant or unwanted 38650, 28472, 5495, 65507, 81588 or 14354 expression or activity. With regards to both prophylactic and therapeutic methods of treatment, such treatments may be specifically tailored or modified, based on knowledge obtained from the field of pharmacogenomics. “Treatment”, as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 38650, 28472, 5495, 65507, 81588 or 14354 molecules of the present invention or 38650, 28472, 5495, 65507, 81588 or 14354 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted 38650, 28472, 5495, 65507, 81588 or 14354 expression or activity, by administering to the subject a 38650, 28472, 5495, 65507, 81588 or 14354 or an agent which modulates 38650, 28472, 5495, 65507, 81588 or 14354 expression or at least one 38650, 28472, 5495, 65507, 81588 or 14354 activity. Subjects at risk for a disease which is caused or contributed to by aberrant or unwanted 38650, 28472, 5495, 65507, 81588 or 14354 expression or activity can be identified by, for example, any or a combination of diagnostic or prognostic assays as described herein. Administration of a prophylactic agent can occur prior to the manifestation of symptoms characteristic of the 38650, 28472, 5495, 65507, 81588 or 14354 aberrance, such that a disease or disorder is prevented or, alternatively, delayed in its progression. Depending on the type of 38650, 28472, 5495, 65507, 81588 or 14354 aberrance, for example, a 38650, 28472, 5495, 65507, 81588 or 14354, 38650, 28472, 5495, 65507, 81588 or 14354 agonist or 38650, 28472, 5495, 65507, 81588 or 14354 antagonist agent can be used for treating the subject. The appropriate agent can be determined based on screening assays described herein.

It is possible that some 38650, 28472, 5495, 65507, 81588 or 14354 disorders can be caused, at least in part, by an abnormal level of gene product, or by the presence of a gene product exhibiting abnormal activity. As such, the reduction in the level and/or activity of such gene products would bring about the amelioration of disorder symptoms.

As discussed, successful treatment of 38650, 28472, 5495, 65507, 81588 or 14354 disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of 38650, 28472, 5495, 65507, 81588 or 14354 disorders. Such molecules can include, but are not limited to peptides, phosphopeptides, small organic or inorganic molecules, or antibodies (including, for example, polyclonal, monoclonal, humanized, anti-idiotypic, chimeric or single chain antibodies, and FAb, F(ab′)₂ and FAb expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules may be utilized in treating or preventing a disease characterized by 38650, 28472, 5495, 65507, 81588 or 14354 expression is through the use of aptamer molecules specific for 38650, 28472, 5495, 65507, 81588 or 14354 protein. Aptamers are nucleic acid molecules having a tertiary structure which permits them to specifically bind to protein ligands (see, e.g., Osborne, et al., Curr. Opin. Chem. Biol. 1997, 1(1): 5-9; and Patel, D. J., Curr. Opin. Chem. Biol. 1997 Jun;1(1):32-46). Since nucleic acid molecules may in many cases be more conveniently introduced into target cells than therapeutic protein molecules may be, aptamers offer a method by which 38650, 28472, 5495, 65507, 81588 or 14354 protein activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of 38650, 28472, 5495, 65507, 81588 or 14354 disorders. For a description of antibodies, see the Antibody section above.

In circumstances wherein injection of an animal or a human subject with a 38650, 28472, 5495, 65507, 81588 or 14354 protein or epitope for stimulating antibody production is harmful to the subject, it is possible to generate an immune response against 38650, 28472, 5495, 65507, 81588 or 14354 through the use of anti-idiotypic antibodies (see, for example, Herlyn, D., Ann. Med. 1999;31(1):66-78;l and Bhattacharya-Chattejee, M., and Foon, K. A., Cancer Treat. Res. 1998;94:51-68). If an anti-idiotypic antibody is introduced into a mammal or human subject, it should stimulate the production of anti-anti-idiotypic antibodies, which should be specific to the 38650, 28472, 5495, 65507, 81588 or 14354 protein. Vaccines directed to a disease characterized by 38650, 28472, 5495, 65507, 81588 or 14354 expression may also be generated in this fashion.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al., (1993, Proc. Natl. Acad. Sci. USA 90:7889-7893).

The identified compounds that inhibit target gene expression, synthesis and/or activity can be administered to a patient at therapeutically effective doses to prevent, treat or ameliorate 38650, 28472, 5495, 65507, 81588 or 14354 disorders. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate 38650, 28472, 5495, 65507, 81588 or 14354 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell, R. J. et al., (1996) Current Opinion in Biotechnology 7:89-94 and in Shea, K. J., (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al., (1993) Nature 361:645-647. Through the use of isotope-labeling, the “free” concentration of compound which modulates the expression or activity of 38650, 28472, 5495, 65507, 81588 or 14354 can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. A rudimentary example of such a “biosensor” is discussed in Kriz, D. et al., (1995) Analytical Chemistry 67:2142-2144.

Another aspect of the invention pertains to methods of modulating 38650, 28472, 5495, 65507, 81588 or 14354 expression or activity for therapeutic purposes. Accordingly, in an exemplary embodiment, the modulatory method of the invention involves contacting a cell with a 38650, 28472, 5495, 65507, 81588 or 14354 or agent that modulates one or more of the activities of 38650, 28472, 5495, 65507, 81588 or 14354 protein activity associated with the cell. An agent that modulates 38650, 28472, 5495, 65507, 81588 or 14354 protein activity can be an agent as described herein, such as a nucleic acid or a protein, a naturally-occurring target molecule of a 38650, 28472, 5495, 65507, 81588 or 14354 protein (e.g., a 38650, 28472, 5495, 65507, 81588 or 14354 substrate or receptor), a 38650, 28472, 5495, 65507, 81588 or 14354 antibody, a 38650, 28472, 5495, 65507, 81588 or 14354 agonist or antagonist, a peptidomimetic of a 38650, 28472, 5495, 65507, 81588 or 14354 agonist or antagonist, or other small molecule.

In one embodiment, the agent stimulates one or more 38650, 28472, 5495, 65507, 81588 or 14354 activities. Examples of such stimulatory agents include active 38650, 28472, 5495, 65507, 81588 or 14354 protein and a nucleic acid molecule encoding 38650, 28472, 5495, 65507, 81588 or 14354. In another embodiment, the agent inhibits one or more 38650, 28472, 5495, 65507, 81588 or 14354 activities. Examples of such inhibitory agents include antisense 38650, 28472, 5495, 65507, 81588 or 14354 nucleic acid molecules, anti-38650, 28472, 5495, 65507, 81588 or 14354 antibodies, and 38650, 28472, 5495, 65507, 81588 or 14354 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 38650, 28472, 5495, 65507, 81588 or 14354 protein or nucleic acid molecule. In one embodiment, the method involves administering an agent (e.g., an agent identified by a screening assay described herein), or combination of agents that modulates (e.g., upregulates or downregulates) 38650, 28472, 5495, 65507, 81588 or 14354 expression or activity. In another embodiment, the method involves administering a 38650, 28472, 5495, 65507, 81588 or 14354 protein or nucleic acid molecule as therapy to compensate for reduced, aberrant, or unwanted 38650, 28472, 5495, 65507, 81588 or 14354 expression or activity.

Stimulation of 38650, 28472, 5495, 65507, 81588 or 14354 activity is desirable in situations in which 38650, 28472, 5495, 65507, 81588 or 14354 is abnormally downregulated and/or in which increased 38650, 28472, 5495, 65507, 81588 or 14354 activity is likely to have a beneficial effect. For example, stimulation of 38650, 28472, 5495, 65507, 81588 or 14354 activity is desirable in situations in which a 38650, 28472, 5495, 65507, 81588 or 14354 is downregulated and/or in which increased 38650, 28472, 5495, 65507, 81588 or 14354 activity is likely to have a beneficial effect. Likewise, inhibition of 38650, 28472, 5495, 65507, 81588 or 14354 activity is desirable in situations in which 38650, 28472, 5495, 65507, 81588 or 14354 is abnormally upregulated and/or in which decreased 38650, 28472, 5495, 65507, 81588 or 14354 activity is likely to have a beneficial effect.

The 38650, 28472, 5495, 65507, 81588 or 14354 molecules can act as novel diagnostic targets and therapeutic agents for controlling one or more of cellular proliferative and/or differentiative disorders, immune disorders, heart disorders, cardiovascular disorders, including endothelial cell disorders, hematopoietic disorders, blood vessel disorders, brain disorders, pain and metabolic disorders, liver disorders and platelet disorders, as described above, as well as disorders associated with bone metabolism or viral diseases.

Aberrant expression and/or activity of 38650, 28472, 5495, 65507, 81588 or 14354 molecules may mediate disorders associated with bone metabolism. “Bone metabolism” refers to direct or indirect effects in the formation or degeneration of bone structures, e.g., bone formation, bone resorption, etc., which may ultimately affect the concentrations in serum of calcium and phosphate. This term also includes activities mediated by 38650, 28472, 5495, 65507, 81588 or 14354 molecules effects in bone cells, e.g. osteoclasts and osteoblasts, that may in turn result in bone formation and degeneration. For example, 38650, 28472, 5495, 65507, 81588 or 14354 molecules may support different activities of bone resorbing osteoclasts such as the stimulation of differentiation of monocytes and mononuclear phagocytes into osteoclasts. Accordingly, 38650, 28472, 5495, 65507, 81588 or 14354 molecules that modulate the production of bone cells can influence bone formation and degeneration, and thus may be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

Additionally, 38650, 28472, 5495, 65507, 81588 or 14354 molecules may play an important role in the etiology of certain viral diseases, including but not limited to, Hepatitis B, Hepatitis C and Herpes Simplex Virus (HSV). Modulators of 38650, 28472, 5495, 65507, 81588 or 14354 activity could be used to control viral diseases. The modulators can be used in the treatment and/or diagnosis of viral infected tissue or virus-associated tissue fibrosis, especially liver and liver fibrosis. Also, 38650, 28472, 5495, 65507, 81588 or 14354 modulators can be used in the treatment and/or diagnosis of virus-associated carcinoma, especially hepatocellular cancer.

Pharmacogenomics

The 38650, 28472, 5495, 65507, 81588 or 14354 molecules of the present invention, as well as agents, or modulators which have a stimulatory or inhibitory effect on 38650, 28472, 5495, 65507, 81588 or 14354 activity (e.g., 38650, 28472, 5495, 65507, 81588 or 14354 gene expression) as identified by a screening assay described herein can be administered to individuals to treat (prophylactically or therapeutically) 38650, 28472, 5495, 65507, 81588 or 14354 associated disorders (e.g., adenosine deaminase, glycoprotease, or seven transmembrane receptor related disorders) associated with aberrant or unwanted 38650, 28472, 5495, 65507, 81588 or 14354 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a 38650, 28472, 5495, 65507, 81588 or 14354 molecule or 38650, 28472, 5495, 65507, 81588 or 14354 modulator as well as tailoring the dosage and/or therapeutic regimen of treatment with a 38650, 28472, 5495, 65507, 81588 or 14354 molecule or 38650, 28472, 5495, 65507, 81588 or 14354 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11) :983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occuring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high-resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a 38650, 28472, 5495, 65507, 81588 or 14354 protein of the present invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a 38650, 28472, 5495, 65507, 81588 or 14354 molecule or 38650, 28472, 5495, 65507, 81588 or 14354 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 38650, 28472, 5495, 65507, 81588 or 14354 molecule or 38650, 28472, 5495, 65507, 81588 or 14354 modulator, such as a modulator identified by one of the exemplary screening assays described herein.

The present invention further provides methods for identifying new agents, or combinations, that are based on identifying agents that modulate the activity of one or more of the gene products encoded by one or more of the 38650, 28472, 5495, 65507, 81588 or 14354 genes of the present invention, wherein these products may be associated with resistance of the cells to a therapeutic agent. Specifically, the activity of the proteins encoded by the 38650, 28472, 5495, 65507, 81588 or 14354 genes of the present invention can be used as a basis for identifying agents for overcoming agent resistance. By blocking the activity of one or more of the resistance proteins, target cells, e.g., cancer cells, will become sensitive to treatment with an agent that the unmodified target cells were resistant to.

Monitoring the influence of agents (e.g., drugs) on the expression or activity of a 38650, 28472, 5495, 65507, 81588 or 14354 protein can be applied in clinical trials. For example, the effectiveness of an agent determined by a screening assay as described herein to increase 38650, 28472, 5495, 65507, 81588 or 14354 gene expression, protein levels, or upregulate 38650, 28472, 5495, 65507, 81588 or 14354 activity, can be monitored in clinical trials of subjects exhibiting decreased 38650, 28472, 5495, 65507, 81588 or 14354 gene expression, protein levels, or downregulated 38650, 28472, 5495, 65507, 81588 or 14354 activity. Alternatively, the effectiveness of an agent determined by a screening assay to decrease 38650, 28472, 5495, 65507, 81588 or 14354 gene expression, protein levels, or downregulate 38650, 28472, 5495, 65507, 81588 or 14354 activity, can be monitored in clinical trials of subjects exhibiting increased 38650, 28472, 5495, 65507, 81588 or 14354 gene expression, protein levels, or upregulated 38650, 28472, 5495, 65507, 81588 or 14354 activity. In such clinical trials, the expression or activity of a 38650, 28472, 5495, 65507, 81588 or 14354 gene, and preferably, other genes that have been implicated in, for example, a 38650, 28472, 5495, 65507, 81588 or 14354-associated disorder can be used as a “read out” or markers of the phenotype of a particular cell.

Other Embodiments

In another aspect, the invention features, a method of analyzing a plurality of capture probes. The method can be used, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence; contacting the array with a 38650, 28472, 5495, 65507, 81588 or 14354, preferably purified, nucleic acid, preferably purified, polypeptide, preferably purified, or antibody, and thereby evaluating the plurality of capture probes. Binding, e.g., in the case of a nucleic acid, hybridization with a capture probe at an address of the plurality, is detected, e.g., by signal generated from a label attached to the 38650, 28472, 5495, 65507, 81588 or 14354 nucleic acid, polypeptide, or antibody.

The capture probes can be a set of nucleic acids from a selected sample, e.g., a sample of nucleic acids derived from a control or non-stimulated tissue or cell.

The method can include contacting the 38650, 28472, 5495, 65507, 81588 or 14354 nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of 38650, 28472, 5495, 65507, 81588 or 14354. Such methods can be used to diagnose a subject, e.g., to evaluate risk for a disease or disorder, to evaluate suitability of a selected treatment for a subject, to evaluate whether a subject has a disease or disorder. 38650, 28472, 5495, 65507, 81588 or 14354 is associated with adenosine deaminase, glycoprotease, or 7TM receptor activity, thus it is useful for disorders associated with abnormal adenosine deaminase, glycoprotease, or 7TM receptor activity.

The method can be used to detect SNPs, as described above.

In another aspect, the invention features, a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express or mis express 38650, 28472, 5495, 65507, 81588 or 14354 or from a cell or subject in which a 38650, 28472, 5495, 65507, 81588 or 14354 mediated response has been elicited, e.g., by contact of the cell with 38650, 28472, 5495, 65507, 81588 or 14354 nucleic acid or protein, or administration to the cell or subject 38650, 28472, 5495, 65507, 81588 or 14354 nucleic acid or protein; contacting the array with one or more inquiry probe, wherein an inquiry probe can be a nucleic acid, polypeptide, or antibody (which is preferably other tha 38650, 28472, 5495, 65507, 81588 or 14354 nucleic acid, polypeptide, or antibody); providing a two dimensional array having a plurality of addresses, each address of the plurality being positionally distinguishable from each other address of the plurality, and each address of the plurality having a unique capture probe, e.g., wherein the capture probes are from a cell or subject which does not express 38650, 28472, 5495, 65507, 81588 or 14354 (or does not express as highly as in the case of the 38650, 28472, 5495, 65507, 81588 or 14354 positive plurality of capture probes) or from a cell or subject which in which a 38650, 28472, 5495, 65507, 81588 or 14354 mediated response has not been elicited (or has been elicited to a lesser extent than in the first sample); contacting the array with one or more inquiry probes (which is preferably other than a 38650, 28472, 5495, 65507, 81588 or 14354 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features, a method of analyzing 38650, 28472, 5495, 65507, 81588 or 14354, e.g., analyzing structure, function, or relatedness to other nucleic acid or amino acid sequences. The method includes: providing a 38650, 28472, 5495, 65507, 81588 or 14354 nucleic acid or amino acid sequence; comparing the 38650, 28472, 5495, 65507, 81588 or 14354 sequence with one or more preferably a plurality of sequences from a collection of sequences, e.g., a nucleic acid or protein sequence database; to thereby analyze 38650, 28472, 5495, 65507, 81588 or 14354.

Preferred databases include GenBank™. The method can include evaluating the sequence identity between a 38650, 28472, 5495, 65507, 81588 or 14354 sequence and a database sequence. The method can be performed by accessing the database at a second site, e.g., over the internet.

In another aspect, the invention features, a set of oligonucleotides, useful, e.g., for identifying SNP's, or identifying specific alleles of 38650, 28472, 5495, 65507, 81588 or 14354. The set includes a plurality of oligonucleotides, each of which has a different nucleotide at an interrogation position, e.g., an SNP or the site of a mutation. In a preferred embodiment, the oligonucleotides of the plurality identical in sequence with one another (except for differences in length). The oligonucleotides can be provided with different labels, such that an oligonucleotides which hybridizes to one allele provides a signal that is distinguishable from an oligonucleotides which hybridizes to a second allele.

This invention is further illustrated by the following examples which should not be construed as limiting. The contents of all references, patents and published patent applications cited throughout this application are incorporated herein by reference.

### EXAMPLES

### Example 1

### Identification and Characterization of Human 38650,28472.5495, 65507, 81588 or 14354 cDNAs

The human 38650 sequence (FIG. 1; SEQ ID NO:1), which is approximately 1680 nucleotides long including untranslated regions, contains a predicted methionine-initiated coding sequence of about 1068 nucleotides (nucleotides 340-1407 of SEQ ID NO:1; SEQ ID NO:3), including the terminal codon. The coding sequence encodes a 355 amino acid protein (SEQ ID NO:2).

The human 28472 sequence (FIG. 8A-B; SEQ ID NO:4), which is approximately 1820 nucleotides long including untranslated regions, contains a predicted methionine-initiated coding sequence of about 1245 nucleotides (nucleotides 146-1390 of SEQ ID NO:4; SEQ ID NO:6), including the terminal codon. The coding sequence encodes a 414 amino acid protein (SEQ ID NO:5).

The human 5495 sequence (FIG. 13; SEQ ID NO:7), which is approximately 1313 nucleotides long including untranslated regions, contains a predicted methionine-initiated coding sequence of about 993 nucleotides (nucleotides 138-1130 of SEQ ID NO:7; SEQ ID NO:9), including the terminal codon. The coding sequence encodes a 330 amino acid protein (SEQ ID NO:8).

The human 65507 sequence (FIG. 17; SEQ ID NO:10), which is approximately 1526 nucleotides long including untranslated regions, contains a predicted methionine-initiated coding sequence of about 1062 nucleotides (nucleotides 139-1200 of SEQ ID NO:10; SEQ ID NO:12), including the terminal codon. The coding sequence encodes a 353 amino acid protein (SEQ ID NO:11).

The human 81588 sequence (FIG. 21A-B; SEQ ID NO:13), which is approximately 1719 nucleotides long including untranslated regions, contains a predicted methionine-initiated coding sequence of about 1125 nucleotides (nucleotides 97-1221 of SEQ ID NO:13; SEQ ID NO:15), including the terminal codon. The coding sequence encodes a 374 amino acid protein (SEQ ID NO:14).

The human 14354 sequence (FIG. 25A-C; SEQ ID NO:16), which is approximately 3068 nucleotides long including untranslated regions, contains a predicted methionine-initiated coding sequence of about 2733 nucleotides (nucleotides 199-2931 of SEQ ID NO:16; SEQ ID NO:18), including the terminal codon. The coding sequence encodes a 910 amino acid protein (SEQ ID NO:17).

### Example 2

### Tissue Distribution of 38650, 28472, 5495, 65507, 81588 or 14354 mRNA

Northern blot hybridizations with various RNA samples can be performed under standard conditions and washed under stringent conditions, i.e., 0.2×SSC at 65° C. A DNA probe corresponding to all or a portion of the 38650 cDNA (SEQ ID NO:1) or 28472 cDNA (SEQ ID NO:4) or 5495 cDNA (SEQ ID NO:7) or 65507 cDNA (SEQ ID NO:10) or 81588 cDNA (SEQ ID NO:13) or 14354 cDNA (SEQ ID NO:16) can be used. The DNA was radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations.

### Example 3

### Gene Expression Analysis

Total RNA was prepared from various human tissues by a single step extraction method using RNA STAT-60 according to the manufacturer's instructions (TelTest, Inc). Each RNA preparation was treated with DNase I (Ambion) at 37° C. for 1 hour. DNAse I treatment was determined to be complete if the sample required at least 38 PCR amplification cycles to reach a threshold level of fluorescence using β-2 microglobulin as an internal amplicon reference. The integrity of the RNA samples following DNase I treatment was confirmed by agarose gel electrophoresis and ethidium bromide staining. After phenol extraction cDNA was prepared from the sample using the SUPERSCRIPT™ Choice System following the manufacturer's instructions (GibcoBRL). A negative control of RNA without reverse transcriptase was mock reverse transcribed for each RNA sample.

Human 38650, 28472, 5495, 81588 or 14354 expression was measured by TaqMan® quantitative PCR (Perkin Elmer Applied Biosystems) in cDNA prepared from a variety of normal and diseased (e.g., cancerous) human tissues or cell lines.

Probes were designed by PrimerExpress software (PE Biosystems) based on the sequence of the human 38650, 28472, 5495, 81588 or 14354 gene. Each human 38650, 28472, 5495, 81588 or 14354 gene probe was labeled using FAM (6-carboxyfluorescein), and the β2-microglobulin reference probe was labeled with a different fluorescent dye, VIC. The differential labeling of the target gene and internal reference gene thus enabled measurement in same well. Forward and reverse primers and the probes for both β2-microglobulin and target gene were added to the TaqMan® Universal PCR Master Mix (PE Applied Biosystems). Although the final concentration of primer and probe could vary, each was internally consistent within a given experiment. A typical experiment contained 200 nM of forward and reverse primers plus 100 nM probe for β-2 microglobulin and 600 nM forward and reverse primers plus 200 nM probe for the target gene. TaqMan matrix experiments were carried out on an ABI PRISM 7700 Sequence Detection System (PE Applied Biosystems). The thermal cycler conditions were as follows: hold for 2 min at 50° C. and 10 min at 95° C., followed by two-step PCR for 40 cycles of 95° C. for 15 sec followed by 60° C. for 1 min.

The following method was used to quantitatively calculate human 38650, 28472, 5495, 81588 or 14354 gene expression in the various tissues relative to β-2 microglobulin expression in the same tissue. The threshold cycle (Ct) value is defined as the cycle at which a statistically significant increase in fluorescence is detected. A lower Ct value is indicative of a higher mRNA concentration. The Ct value of the human 38650, 28472, 5495, 81588 or 14354 gene is normalized by subtracting the Ct value of the β-2 microglobulin gene to obtain a ΔCt value using the following formula: ΔCt=Ct_{human 59914 and 59921}-Ct_{β-2 microglobulin}. Expression is then calibrated against a cDNA sample showing a comparatively low level of expression of the human 38650, 28472, 5495, 81588 or 14354 gene. The ΔCt value for the calibrator sample is then subtracted from ΔCt for each tissue sample according to the following formula: ΔΔCt=ΔCt−ₛₐₘₚₗₑ−ΔCt−_{calibrator}. Relative expression is then calculated using the arithmetic formula given by 2−ΔΔCt. Expression of the target human 38650, 28472, 5495, 81588 or 14354 gene in each of the tissues tested is then graphically represented as discussed in more detail below.

TaqMan real-time quantitative RT-PCR is used to detect the presence of RNA transcript corresponding to human 38650 relative to a no template control in a panel of human tissues or cells. Table 1 indicates highest expression levels in coronary smooth muscle cells, erythroid cells, and normal brain cortex tissue. There is increased expression of human 38650 in heart tissue with congestive heart failure compared to normal heart tissue. There is also increased expression levels of 38650 in ovary tumor, prostate tumor, colon tumor lung tumor tissue and fibrotic liver compared to normal ovary, prostate, colon, lung, and liver tissue. In addition, there is decreased expression levels of 38650 in diseased aorta, and breast tumor tissue compared to normal aorta and breast tissue.

TABLE 1

| Tissue Type | Mean | β 2 Mean | ∂∂ Ct | Expression |
|---|---|---|---|---|
| Artery normal | 32.45 | 22.89 | 9.56 | 1.3248 |
| Aorta diseased | 35.85 | 23.2 | 12.65 | 0 |
| Vein normal | 34.94 | 21.59 | 13.34 | 0.0964 |
| Coronary SMC | 30.44 | 25.17 | 5.27 | 25.9162 |
| HUVEC | 31.67 | 23 | 8.66 | 2.4636 |
| Hemangioma | 33.35 | 21.85 | 11.51 | 0.3441 |
| Heart normal | 29.72 | 21.11 | 8.61 | 2.5682 |
| Heart CHF | 26.52 | 20.75 | 5.77 | 18.3255 |
| Kidney | 29.12 | 21.3 | 7.82 | 4.4253 |
| Adipose normal | 35.98 | 23.5 | 12.48 | 0 |
| Pancreas | 33.41 | 24.23 | 9.18 | 1.724 |
| primary osteoblasts | 34.17 | 21.23 | 12.94 | 0.1277 |
| Osteoclasts (diff) | 35.8 | 18.65 | 17.16 | 0 |
| Skin normal | 34.94 | 23.06 | 11.89 | 0.2644 |
| Spinal cord normal | 36.3 | 22.29 | 14.01 | 0 |
| Brain Cortex normal | 29.52 | 23.95 | 5.58 | 20.9051 |
| Brain Hypothalamus normal | 31.09 | 24.23 | 6.86 | 8.6086 |
| Nerve | 31.57 | 22.45 | 9.12 | 1.7972 |
| DRG (Dorsal Root Ganglion) | 31.07 | 22.85 | 8.22 | 3.3422 |
| Breast normal | 32.2 | 22.4 | 9.8 | 1.1179 |
| Breast tumor | 33.7 | 22.13 | 11.57 | 0.3289 |
| Ovary normal | 31.28 | 21.73 | 9.54 | 1.3387 |
| Ovary Tumor | 30.84 | 21.93 | 8.91 | 2.0788 |
| Prostate Normal | 29.43 | 20.18 | 9.26 | 1.6367 |
| Prostate Tumor | 30.07 | 21.2 | 8.88 | 2.1299 |
| Salivary glands | 35.56 | 20.82 | 14.74 | 0 |
| Colon normal | 34.41 | 20.36 | 14.05 | 0.0592 |
| Colon Tumor | 33.4 | 23.23 | 10.17 | 0.868 |
| Lung normal | 35.98 | 20.72 | 15.26 | 0 |
| Lung tumor | 27.62 | 20.73 | 6.89 | 8.4315 |
| Lung COPD | 32.35 | 19.36 | 13 | 0.1221 |
| Colon IBD | 34 | 18.91 | 15.09 | 0.0287 |
| Liver normal | 33.03 | 21.67 | 11.36 | 0.3805 |
| Liver fibrosis | 32.75 | 22.9 | 9.85 | 1.0836 |
| Spleen normal | 35.92 | 21.74 | 14.19 | 0 |
| Tonsil normal | 30.14 | 18.05 | 12.09 | 0.2294 |
| Lymph node normal | 32.55 | 20.55 | 11.99 | 0.245 |
| Small intestine normal | 34.12 | 21.66 | 12.46 | 0.1781 |
| Macrophages | 40 | 18.77 | 21.23 | 0 |
| Synovium | 38.62 | 21.86 | 16.76 | 0 |
| BM-MNC | 40 | 21.01 | 18.99 | 0 |
| Activated PBMC | 32.12 | 18.82 | 13.3 | 0.0995 |
| Neutrophils | 33.76 | 19.7 | 14.07 | 0.0583 |
| Megakaryocytes | 31.26 | 19.72 | 11.55 | 0.3347 |
| Erythroid | 28.41 | 22.99 | 5.42 | 23.2762 |
| positive control | 27.41 | 22.2 | 5.21 | 27.0168 |
| Skeletal Muscle | 33.15 | 26.57 | 6.59 | 10.4164 |

TaqMan real-time quantitative RT-PCR is used to detect the presence of RNA transcript corresponding to human 38650 relative to a no template control in a panel of human tissues or cells. Table 2 again indicates increased expression in heart with congestive heart failure compared to normal heart tissue. There is also increased expression in ovary tumor, prostate tumor, colon tumor lung tumor tissue and fibrotic liver compared to normal ovary, prostate, colon, lung, and liver tissue. In addition, there is decreased expression levels of 38650 in diseased aorta, and breast tumor tissue compared to normal aorta and breast tissue.

TABLE 2

| Tissue Type | Mean | β 2 Mean | ∂∂ Ct | Expression |
|---|---|---|---|---|
| Artery normal | 33.66 | 23.32 | 8.34 | 3.0861 |
| Aorta diseased | 35.45 | 23.59 | 9.87 | 0 |
| Vein normal | 35.03 | 21.53 | 11.51 | 0 |
| Coronary SMC | 31.57 | 22.2 | 7.37 | 6.0452 |
| HUVEC | 32.5 | 22.66 | 7.84 | 4.3493 |
| Hemangioma | 32.67 | 21.07 | 9.61 | 1.2841 |
| Heart normal | 31.29 | 21.82 | 7.46 | 5.6796 |
| Heart CHF | 27.93 | 21.16 | 4.76 | 36.7783 |
| Kidney | 30.06 | 21.59 | 6.47 | 11.2807 |
| Skeletal Muscle | 37.03 | 28.4 | 6.63 | 0 |
| Adipose normal | 34.37 | 21.93 | 10.44 | 0.7224 |
| Pancreas | 33.22 | 23.44 | 7.78 | 4.5497 |
| primary osteoblasts | 35.38 | 21.97 | 11.4 | 0 |
| Osteoclasts (diff) | 34.85 | 18.88 | 13.98 | 0.0621 |
| Skin normal | 35.66 | 23.2 | 10.46 | 0 |
| Spinal cord normal | 34.48 | 22.06 | 10.43 | 0.7274 |
| Brain Cortex normal | 31.03 | 23.45 | 5.58 | 20.9777 |
| Brain Hypothalamus normal | 32.72 | 23.45 | 7.26 | 6.5241 |
| Nerve | 34.5 | 23.21 | 9.29 | 1.5975 |
| DRG (Dorsal Root Ganglion) | 33.51 | 23.37 | 8.13 | 3.5573 |
| Breast normal | 33.65 | 22.27 | 9.37 | 1.5113 |
| Breast tumor | 34.85 | 22.02 | 10.84 | 0.5456 |
| Ovary normal | 32.91 | 21.03 | 9.88 | 1.0649 |
| Ovary Tumor | 32.24 | 21.13 | 9.12 | 1.8035 |
| Prostate Normal | 32.5 | 20.82 | 9.68 | 1.2191 |
| Prostate Tumor | 32.42 | 21.76 | 8.66 | 2.4722 |
| Salivary glands | 35.33 | 21 | 12.32 | 0 |
| Colon normal | 34.03 | 19.82 | 12.21 | 0.2118 |
| Colon Tumor | 35.08 | 22.36 | 10.72 | 0 |
| Lung normal | 34.35 | 19.38 | 12.97 | 0.1246 |
| Lung tumor | 30.2 | 21.47 | 6.72 | 9.4531 |
| Lung COPD | 33.54 | 19.81 | 11.73 | 0.2934 |
| Colon IBD | 33.73 | 18.95 | 12.79 | 0.1417 |
| Liver normal | 33.38 | 21.28 | 10.11 | 0.908 |
| Liver fibrosis | 32.91 | 22.06 | 8.85 | 2.1671 |
| Spleen normal | 34.03 | 21.05 | 10.99 | 0.4917 |
| Tonsil normal | 31.52 | 18.55 | 10.97 | 0.5003 |
| Lymph node normal | 34.06 | 20.99 | 11.07 | 0.4668 |
| Small intestine normal | 34.59 | 21.52 | 11.06 | 0.4684 |
| Macrophages | 36.8 | 18.3 | 16.5 | 0 |
| Synovium | 35.31 | 20.86 | 12.45 | 0 |
| BM-MNC | 37.5 | 20.07 | 15.43 | 0 |
| Activated PBMC | 33.94 | 19.13 | 12.81 | 0.1393 |
| Neutrophils | 34.42 | 20.16 | 12.27 | 0.2025 |
| Megakaryocytes | 31.09 | 20.11 | 8.98 | 1.9804 |
| Erythroid | 29.44 | 22.77 | 4.67 | 39.2817 |
| positive control | 28.26 | 21.41 | 4.85 | 34.674 |

TaqMan real-time quantitative RT-PCR is used to detect the presence of RNA transcript corresponding to human 38650 in a panel of human tissues or cells. Table 3 indicates increased expression of 38650 in diseased heart compared to normal heart. There also appears to be a slight decrease in expression of 38650 in kidneys taken from subjects with hypertension versus normal kidneys.

TABLE 3

| | | β 2 | | |
|---|---|---|---|---|
| Tissue Type | Mean | Mean | ∂∂ Ct | Expression |
| H/Fetal Heart/normal/BWH 4 | 28.22 | 21.97 | 6.25 | 13.0935 |
| H/Heart/Normal/Atrium/MPI 1097 | 27.36 | 20.77 | 6.58 | 10.4526 |
| H/Heart/Normal/Ventricle/PIT 272 | 25.69 | 17.74 | 7.95 | 4.044 |
| H/Heart/Normal/Ventricle/PIT 206 | 29.18 | 20.73 | 8.45 | 2.8695 |
| H/Heart/Normal/Ventricle/PIT 204 | 25.74 | 18.57 | 7.17 | 6.9682 |
| H/Heart/Normal/Ventricle/PIT 205 | 25.34 | 18.28 | 7.07 | 7.4683 |
| H/Heart/Diseased/Ventricle/ELI 5 | 22.06 | 16.68 | 5.38 | 24.0137 |
| H/Heart/Diseased/Ventricle/PIT 16 | 23.98 | 18.41 | 5.58 | 20.9051 |
| H/Heart/Diseased/Ventricle/PIT 1 | 27.39 | 21.91 | 5.47 | 22.5614 |
| H/Heart/Diseased/Ventricle/PIT 14 | 25.82 | 20.01 | 5.82 | 17.7628 |
| H/Kidney/normal/NDR 171 | 28.57 | 21.09 | 7.47 | 5.6403 |
| H/Kidney/normal/NDR 179 | 27.75 | 19.98 | 7.76 | 4.5973 |
| H/Kidney/normal/PIT 289 | 30.07 | 23.46 | 6.61 | 10.2374 |
| H/Kidney/normal/PIT 351 | 25.93 | 19.21 | 6.72 | 9.4531 |
| H/Kidney/normal/PIT 353 | 27.41 | 19.25 | 8.16 | 3.4962 |
| H/Kidney/HT/NDR 233 | 26.35 | 18.57 | 7.79 | 4.534 |
| H/Kidney/HT/NDR 224 | 28.32 | 19.18 | 9.14 | 1.7664 |
| H/Kidney/HT/CHT 1176 | 30.34 | 22.52 | 7.83 | 4.41 |
| H/Kidney/HT/NDR 252 | 30.78 | 23.05 | 7.73 | 4.7102 |
| H/Kidney/HT/CHT 762 | 29.87 | 21.54 | 8.34 | 3.0968 |
| H/Skeletal Muscle/Normal/PIT 915 | 28.25 | 22.32 | 5.93 | 16.3451 |
| H/Skeletal Muscle/Normal/PIT 685 | 29.57 | 23.36 | 6.2 | 13.6024 |
| H/Skeletal Muscle/Normal/PIT 428 | 29.02 | 23.61 | 5.41 | 23.6011 |
| H/Liver/Normal/MPI 146 | 26.61 | 17.5 | 9.12 | 1.8035 |
| H/Liver/Normal/CHT 339 | 32.02 | 21.64 | 10.38 | 0.753 |
| H/Liver/Normal/CHT 1237 | 32.98 | 21.58 | 11.4 | 0.3688 |

TaqMan real-time quantitative RT-PCR is used to detect the presence of RNA transcript corresponding to human 38650 in a panel of human tissues or cells. Table 4 indicates the highest level of 38650 expression is seen in human umbilical vein endothelial cells (HUVEC) that is shear/static pooled.

TABLE 4

| | | β 2 | | Expres- |
|---|---|---|---|---|
| Tissue Type | Mean | Mean | ∂∂ Ct | sion |
| Coronary SMC | 29.57 | 21.21 | 8.37 | 3.0331 |
| Huvec NS | 30.97 | 20.61 | 10.37 | 0.7583 |
| Huvec Shear/static pooled | 29.63 | 21.79 | 7.84 | 4.3644 |
| H/Adipose/PIT 695 | 29.84 | 19.93 | 9.91 | 1.0358 |
| H/Internal Mam Artery/Normal/AMC 263 | 31.12 | 21.18 | 9.94 | 1.018 |
| H/Internal Mam Artery/Normal/AMC 347 | 32.12 | 22.18 | 9.94 | 1.0216 |
| H/Internal Mam Artery/Normal/AMC 350 | 33.9 | 22.7 | 11.21 | 0.4236 |
| H/Internal Mam Artery/Normal/AMC 352 | 30.99 | 22.08 | 8.91 | 2.0788 |
| H/Artery/Normal/PIT 1180 | 29.68 | 19.57 | 10.11 | 0.9049 |
| H/Artery/normal/PIT 912 | 31.36 | 21.85 | 9.51 | 1.3763 |
| H/Artery/normal/NDR 352 | 29.97 | 20.96 | 9.01 | 1.9396 |
| H/Aorta/Diseased/PIT 710 | 30.27 | 21.8 | 8.47 | 2.8104 |
| H/Aorta/Diseased/PIT 712 | 31.18 | 20.89 | 10.3 | 0.796 |
| H/Aorta/Diseased/PIT 732 | 30.77 | 21.63 | 9.14 | 1.7725 |
| H/Artery/Diseased/iliac/NDR 753 | 30.3 | 20.62 | 9.68 | 1.2191 |
| H/Artery/Diseased/Tibial/PIT 679 | 29.99 | 20.24 | 9.75 | 1.1613 |
| H/Vein/Normal/PIT 1010 | 30.81 | 21.11 | 9.7 | 1.2023 |
| H/Vein/Normal/NDR 239 | 30.65 | 20.73 | 9.91 | 1.0358 |
| H/Vein/Normal/AMC 130 | 30.75 | 20.93 | 9.82 | 1.1025 |
| H/Vein/Normal/AMC 131 | 31.97 | 21.95 | 10.03 | 0.9598 |
| H/Vein/Normal/AMC 137 | 30.16 | 20.77 | 9.39 | 1.4905 |
| H/Vein/Normal/AMC 153 | 29.7 | 20.26 | 9.44 | 1.4397 |
| H/Vein/Normal/AMC 176 | 30.19 | 20.42 | 9.77 | 1.1453 |
| H/Vein/Normal/AMC 177 | 31.3 | 21.83 | 9.46 | 1.415 |
| H/Vein/Normal/AMC 178 | 32.07 | 21.59 | 10.48 | 0.7026 |
| H/Vein/Normal/AMC 182 | 30.82 | 20.02 | 10.8 | 0.5609 |
| H/Vein/Normal/AMC 190 | 31.39 | 21.05 | 10.34 | 0.7742 |
| H/Vein/Normal/AMC 192 | 32.24 | 22.42 | 9.82 | 1.1025 |
| H/Vein/Normal/AMC 195 | 31.36 | 21.87 | 9.49 | 1.3859 |
| H/Vein/Normal/AMC 211 | 31.29 | 21.38 | 9.9 | 1.043 |
| H/Artery/normal/AMC 150 | 35.37 | 24.27 | 11.1 | 0 |
| M/Aorta/Normal/PRI 286 | 32.41 | 19.93 | 12.48 | 0.175 |
| M/Vein/Normal/PRI 328 | 35.57 | 22.48 | 13.1 | 0 |
| M/Vein/Normal/PRI 230 | 34.86 | 24.5 | 10.36 | 0.7609 |
| M/Aorta/Diseased/CAR 1216 | 35.08 | 22.66 | 12.41 | 0 |
| M/Aorta/Diseased/CAR 1237 | 32.52 | 25.07 | 7.46 | 5.6796 |
| M/Aorta/Diseased/CAR 1192 | 33.58 | 24.78 | 8.8 | 2.2436 |
| M/Aorta/Diseased/CAR 1196 | 31.83 | 24.88 | 6.95 | 8.088 |
| M/Artery/Diseased/CAR 1174 | 31.48 | 25.88 | 5.59 | 20.6889 |
| M/Artery/Diseased/CAR 1175 | 31.33 | 20.81 | 10.53 | 0.6787 |

TaqMan real-time quantitative RT-PCR is used to detect the presence of RNA transcript corresponding to human 28472 relative to a no template control in a panel of human tissues or cells. Table 5 indicates the highest level of 28472 expression is seen in coronary smooth muscle cells and normal brain cortex. Table 5 also shows that there is upregulated expression of 28472 in heart with congestive heart failure, prostate tumor, colon tumor, and lung tumor tissue, as compared to normal heart, prostate, colon and lung tissue. Also, there is downregulated expression of 28472 in diseased aorta, breast tumor, and ovary tumor tissue, as compared to normal aorta, breast, and ovary tissue.

TABLE 5

| Tissue Type | Mean | β 2 Mean | ∂∂ Ct | Expression |
|---|---|---|---|---|
| Artery normal | 32.6 | 22.98 | 9.62 | 1.2753 |
| Aorta diseased | 34.24 | 23.04 | 11.2 | 0.4251 |
| Vein normal | 32.64 | 21.02 | 11.62 | 0.3177 |
| Coronary SMC | 28.31 | 24.01 | 4.3 | 50.7658 |
| HUVEC | 28.77 | 22.4 | 6.38 | 12.0485 |
| Hemangioma | 31.94 | 21.08 | 10.86 | 0.538 |
| Heart normal | 32.91 | 21.09 | 11.82 | 0.2766 |
| Heart CHF | 27.86 | 20.52 | 7.34 | 6.1722 |
| Kidney | 29.84 | 21.09 | 8.74 | 2.3307 |
| Skeletal Muscle | 30.36 | 23.82 | 6.55 | 10.6722 |
| Adipose normal | 35.19 | 22.25 | 12.95 | 0 |
| Pancreas | 33.38 | 22.89 | 10.5 | 0.6905 |
| primary osteoblasts | 34.72 | 21.2 | 13.52 | 0.0854 |
| Osteoclasts (diff) | 35.05 | 18.38 | 16.67 | 0 |
| Skin normal | 34.27 | 22.97 | 11.3 | 0.398 |
| Spinal cord normal | 32.74 | 22.25 | 10.49 | 0.6953 |
| Brain Cortex normal | 28.89 | 23.34 | 5.55 | 21.3444 |
| Brain Hypothalamus normal | 30.22 | 23.11 | 7.11 | 7.239 |
| Nerve | 33.68 | 22.55 | 11.14 | 0.4447 |
| DRG (Dorsal Root Ganglion) | 31.59 | 22.89 | 8.71 | 2.3963 |
| Breast normal | 30.82 | 22.18 | 8.64 | 2.5067 |
| Breast tumor | 31 | 21.87 | 9.13 | 1.7848 |
| Ovary normal | 28.18 | 21.56 | 6.62 | 10.1667 |
| Ovary Tumor | 30.78 | 21.61 | 9.17 | 1.736 |
| Prostate Normal | 33.1 | 20.81 | 12.29 | 0.1997 |
| Prostate Tumor | 30.24 | 21.26 | 8.98 | 1.9804 |
| Salivary glands | 33.03 | 20.6 | 12.44 | 0.1806 |
| Colon normal | 34.27 | 19.85 | 14.42 | 0.0456 |
| Colon Tumor | 30.59 | 20.57 | 10.02 | 0.9665 |
| Lung normal | 33.98 | 19.68 | 14.29 | 0.0499 |
| Lung tumor | 28.18 | 21 | 7.18 | 6.8961 |
| Lung COPD | 32.28 | 19.29 | 12.99 | 0.1225 |
| Colon IBD | 34.17 | 18.64 | 15.54 | 0.0211 |
| Liver normal | 34.72 | 21.26 | 13.46 | 0.0891 |
| Liver fibrosis | 34.95 | 23.17 | 11.79 | 0.2834 |
| Spleen normal | 34.2 | 21.15 | 13.06 | 0.1171 |
| Tonsil normal | 31.09 | 18.15 | 12.95 | 0.1268 |
| Lymph node normal | 31.9 | 20.11 | 11.78 | 0.2844 |
| Small intestine normal | 35.82 | 21.52 | 14.3 | 0 |
| Skin-Decubitus | 36.63 | 22.67 | 13.97 | 0 |
| Synovium | 37.45 | 21.27 | 16.18 | 0 |
| BM-MNC | 39.72 | 20.3 | 19.43 | 0 |
| Activated PBMC | 32.06 | 18.61 | 13.45 | 0.0894 |
| Neutrophils | 32.6 | 19.68 | 12.93 | 0.1281 |
| Megakaryocytes | 30.15 | 19.73 | 10.41 | 0.7324 |
| Erythroid | 31.61 | 22.72 | 8.89 | 2.1006 |

TaqMan real-time quantitative RT-PCR is used to detect the presence of RNA transcript corresponding to human 5495 relative to a no template control in a panel of human tissues or cells. Table 6 indicates the highest level of 5495 expression is seen in normal aorta tissue. Table 6 also shows that there is downregulated expression of 5495 in diseased aorta, heart with congestive heart failure, breast tumor, and lung tumor tissue, as compared to normal aorta, heart, breast, and lung tissue.

TABLE 6

| Tissue Type | Mean | β 2 Mean | ∂∂ Ct | Expression |
|---|---|---|---|---|
| Artery normal | 32.63 | 20.48 | 11.15 | 0.4385 |
| Aorta diseased | 40 | 22.5 | 16.5 | 0 |
| Vein normal | 34.2 | 19.91 | 13.28 | 0.1005 |
| Coronary SMC | 40 | 20.54 | 18.47 | 0 |
| HUVEC | 35.91 | 20.68 | 14.23 | 0 |
| Hemangioma | 34.49 | 19.66 | 13.82 | 0.0689 |
| Heart normal | 34.38 | 20.13 | 13.26 | 0.1023 |
| Heart CHF | 35.22 | 20.79 | 13.43 | 0 |
| Kidney | 36.19 | 20.59 | 14.61 | 0 |
| Liver normal | 40 | 19.64 | 19.36 | 0 |
| Small intestine normal | 39.47 | 20.29 | 18.18 | 0 |
| Adipose normal | 32.17 | 19.43 | 11.74 | 0.2924 |
| Pancreas | 35.41 | 21.95 | 12.46 | 0 |
| primary osteoblasts | 37.7 | 19.47 | 17.23 | 0 |
| Bladder-Female normal | 33.84 | 19.59 | 13.26 | 0.1019 |
| Adrenal Gland normal | 40 | 19.21 | 19.79 | 0 |
| Pituitary Gland normal | 40 | 19.82 | 19.18 | 0 |
| Spinal cord normal | 40 | 20.95 | 18.05 | 0 |
| Brain Cortex normal | 38.59 | 22.2 | 15.38 | 0 |
| Brain Hypothalamus normal | 38.88 | 21.07 | 16.81 | 0 |
| Nerve | 40 | 20.44 | 18.56 | 0 |
| DRG (Dorsal Root Ganglion) | 38.16 | 20.93 | 16.23 | 0 |
| Breast normal | 34.92 | 20.77 | 13.15 | 0.1096 |
| Breast tumor/IDC | 39.1 | 19.5 | 18.61 | 0 |
| Ovary normal | 36.2 | 19.91 | 15.3 | 0 |
| Ovary Tumor | 35.98 | 19.59 | 15.4 | 0 |
| Prostate BPH | 37.88 | 19.47 | 17.41 | 0 |
| Prostate Adenocarcinoma | 40 | 20.31 | 18.7 | 0 |
| Colon normal | 37.94 | 19.18 | 17.75 | 0 |
| Colon Adenocarcinoma | 34.42 | 21.64 | 11.79 | 0.2824 |
| Lung normal | 34.58 | 18.18 | 15.4 | 0.0231 |
| Lung tumor | 40 | 20.57 | 18.43 | 0 |
| Lung COPD | 37.35 | 18.54 | 17.82 | 0 |
| Colon IBD | 40 | 20.02 | 18.98 | 0 |
| Synovium | 38.87 | 19.14 | 18.73 | 0 |
| Tonsil normal | 34.92 | 18.41 | 15.51 | 0.0214 |
| Lymph node normal | 37.51 | 20.05 | 16.46 | 0 |
| Liver fibrosis | 37.02 | 20.61 | 15.4 | 0 |
| Spleen normal | 40 | 18.44 | 20.56 | 0 |
| Macrophages | 40 | 17.02 | 21.98 | 0 |
| Progenitors (erythroid, megakaryocyte, neutrophil) | 37.02 | 19.51 | 16.51 | 0 |
| Megakaryocytes | 33.17 | 19.1 | 13.07 | 0.1159 |
| Activated PBMC | 35.9 | 16.65 | 18.25 | 0 |
| Neutrophils | 40 | 18.39 | 20.61 | 0 |
| Erythroid | 40 | 21.18 | 17.82 | 0 |
| positive control | 34.31 | 21.35 | 11.96 | 0.251 |
| Skeletal Muscle | 33.95 | 21.95 | 11.01 | 0.4866 |

TaqMan real-time quantitative RT-PCR is used to detect the presence of RNA transcript corresponding to human 5495 relative to a no template control in a panel of human tissues or cells. Data from RNA transcript expression are shown in Table 7.

TABLE 7

| Tissue Type | Mean | β 2 Mean | ∂∂ Ct | Expression |
|---|---|---|---|---|
| Artery normal | 40 | 22.05 | 17.95 | 0 |
| Aorta diseased | 40 | 22.48 | 17.52 | 0 |
| Vein normal | 39.7 | 20.09 | 19.62 | 0 |
| Coronary SMC | 40 | 20.7 | 19.31 | 0 |
| HUVEC | 40 | 20.77 | 19.23 | 0 |
| Hemangioma | 39.25 | 19.48 | 19.77 | 0 |
| Heart normal | 40 | 20.77 | 19.23 | 0 |
| Heart CHF | 39.56 | 20.5 | 19.06 | 0 |
| Kidney | 40 | 20.12 | 19.88 | 0 |
| Skeletal Muscle | 37.28 | 21.68 | 15.6 | 0 |
| Adipose normal | 36.78 | 19.71 | 17.07 | 0 |
| Pancreas | 40 | 21.73 | 18.27 | 0 |
| primary osteoblasts | 40 | 19.45 | 20.55 | 0 |
| Osteoclasts (diff) | 40 | 17.27 | 22.73 | 0 |
| Skin normal | 40 | 21.46 | 18.54 | 0 |
| Spinal cord normal | 40 | 20.82 | 19.18 | 0 |
| Brain Cortex normal | 40 | 21.59 | 18.41 | 0 |
| Brain Hypothalamus normal | 40 | 22.4 | 17.6 | 0 |
| Nerve | 40 | 21.25 | 18.75 | 0 |
| DRG (Dorsal Root Ganglion) | 40 | 20.82 | 19.18 | 0 |
| Breast normal | 40 | 20.52 | 19.48 | 0 |
| Breast tumor | 40 | 20.84 | 19.16 | 0 |
| Ovary normal | 40 | 19.86 | 20.14 | 0 |
| Ovary Tumor | 40 | 18.52 | 21.48 | 0 |
| Prostate Normal | 40 | 19.68 | 20.32 | 0 |
| Prostate Tumor | 40 | 20.18 | 19.82 | 0 |
| Salivary glands | 38 | 20.04 | 17.96 | 0 |
| Colon normal | 39.62 | 19.02 | 20.6 | 0 |
| Colon Tumor | 39.8 | 21.56 | 18.24 | 0 |
| Lung normal | 38.88 | 18.04 | 20.84 | 0 |
| Lung tumor | 40 | 20.24 | 19.76 | 0 |
| Lung COPD | 40 | 18.24 | 21.76 | 0 |
| Colon IBD | 40 | 18.3 | 21.7 | 0 |
| Liver normal | 40 | 19.98 | 20.02 | 0 |
| Liver fibrosis | 40 | 20.3 | 19.7 | 0 |
| Spleen normal | 40 | 18.32 | 21.68 | 0 |
| Tonsil normal | 39.06 | 18.5 | 20.56 | 0 |
| Lymph node normal | 40 | 20.05 | 19.95 | 0 |
| Small intestine normal | 40 | 20.22 | 19.79 | 0 |
| Macrophages | 39.7 | 17.02 | 22.68 | 0 |
| Synovium | 40 | 19.13 | 20.87 | 0 |
| BM-MNC | 40 | 18.23 | 21.77 | 0 |
| Activated PBMC | 40 | 17.91 | 22.09 | 0 |
| Neutrophils | 40 | 17.89 | 22.11 | 0 |
| Megakaryocytes | 40 | 18.59 | 21.41 | 0 |
| Erythroid | 40 | 20.15 | 19.85 | 0 |
| positive control | 36.61 | 21.15 | 15.46 | 0 |

TaqMan real-time quantitative RT-PCR is used to detect the presence of RNA transcript corresponding to human 81588 relative to a no template control in a panel of human tissues or cells. Table 8 indicates the highest level of 81588 expression is seen in normal brain cortex. Table 8 also shows that there is downregulated expression of 81588 in heart with congestive heart failure, colon tumor, and ovary tumor tissue, as compared to normal heart, colon, and ovary tissue.

TABLE 8

| Tissue Type | Mean | β 2 Mean | ∂∂ Ct | Expression |
|---|---|---|---|---|
| Artery normal | 35.59 | 24.91 | 9.23 | 0 |
| Aorta diseased | 37.53 | 24.61 | 11.49 | 0 |
| Vein normal | 38.38 | 22.45 | 14.49 | 0 |
| Coronary SMC | 35.64 | 24.94 | 9.27 | 0 |
| HUVEC | 36.09 | 23.57 | 11.09 | 0 |
| Hemangioma | 35.44 | 21.95 | 12.06 | 0 |
| Heart normal | 34.37 | 22.61 | 10.32 | 0.7823 |
| Heart CHF | 36.7 | 22.11 | 13.15 | 0 |
| Kidney | 37.71 | 22.42 | 13.86 | 0 |
| Skeletal Muscle | 38.97 | 25.51 | 12.03 | 0 |
| Adipose normal | 38.85 | 22.94 | 14.48 | 0 |
| Pancreas | 34.74 | 23.95 | 9.36 | 1.5271 |
| primary osteoblasts | 38.8 | 22.98 | 14.39 | 0 |
| Osteoclasts (diff) | 40 | 19.8 | 18.77 | 0 |
| Skin normal | 36.56 | 24.18 | 10.96 | 0 |
| Spinal cord normal | 37.49 | 23.15 | 12.91 | 0 |
| Brain Cortex normal | 33.84 | 24.54 | 7.88 | 4.2598 |
| Brain Hypothalamus normal | 37 | 24.36 | 11.21 | 0 |
| Nerve | 36.61 | 24.15 | 11.03 | 0 |
| DRG (Dorsal Root Ganglion) | 34.2 | 24.18 | 8.59 | 2.6041 |
| Breast normal | 36.32 | 23.34 | 11.54 | 0 |
| Breast tumor | 37.52 | 23.07 | 13.01 | 0 |
| Ovary normal | 33.17 | 22.6 | 9.13 | 1.7787 |
| Ovary Tumor | 40 | 22.56 | 16.01 | 0 |
| Prostate Normal | 35.88 | 22.74 | 11.71 | 0 |
| Prostate Tumor | 35.31 | 22.63 | 11.26 | 0 |
| Salivary glands | 38.27 | 29.92 | 6.91 | 0 |
| Colon normal | 33.31 | 20.51 | 11.36 | 0.3805 |
| Colon Tumor | 37.15 | 23.7 | 12.02 | 0 |
| Lung normal | 36.56 | 20.36 | 14.77 | 0 |
| Lung tumor | 36.16 | 22.77 | 11.97 | 0 |
| Lung COPD | 36.91 | 20.7 | 14.77 | 0 |
| Colon IBD | 34.94 | 19.96 | 13.54 | 0.084 |
| Liver normal | 35.31 | 22.41 | 11.48 | 0 |
| Liver fibrosis | 36.66 | 24.02 | 11.21 | 0 |
| Spleen normal | 33.38 | 21.96 | 9.98 | 0.9902 |
| Tonsil normal | 32.01 | 19.42 | 11.15 | 0.4385 |
| Lymph node normal | 32.23 | 21.48 | 9.31 | 1.57 |
| Small intestine normal | 35.81 | 22.51 | 11.87 | 0 |
| Macrophages | 37.66 | 19.27 | 16.95 | 0 |
| Synovium | 35.69 | 21.93 | 12.32 | 0 |
| BM-MNC | 38.76 | 21.04 | 16.29 | 0 |
| Activated PBMC | 40 | 30.07 | 8.5 | 0 |
| Neutrophils | 40 | 21.04 | 17.53 | 0 |
| Megakaryocytes | 37.51 | 29.96 | 6.11 | 0 |
| Erythroid | 40 | 23.77 | 14.8 | 0 |
| positive control | 32.35 | 22.34 | 8.59 | 2.6041 |

TaqMan real-time quantitative RT-PCR is used to detect the presence of RNA transcript corresponding to human 14354 relative to a no template control in a panel of human tissues or cells. Table 9 indicates the highest level of 14354 expression is seen in kidney tissue. Table 9 also shows that there is upregulated expression of 14354 in ovary tumor, prostate tumor, colon tumor, lung tumor, and fibrotic liver tissue, as compared to normal ovary, prostate, colon, lung and liver tissue.

TABLE 9

| Tissue Type | Mean | β 2 Mean | ∂∂ Ct | Expression |
|---|---|---|---|---|
| Artery normal | 35.34 | 21.67 | 13.67 | 0 |
| Aorta diseased | 40 | 21.59 | 18.41 | 0 |
| Vein normal | 38.79 | 20.02 | 18.77 | 0 |
| Coronary SMC | 35.08 | 22.15 | 12.94 | 0 |
| HUVEC | 37.65 | 20.5 | 17.15 | 0 |
| Hemangioma | 35.46 | 18.93 | 16.52 | 0 |
| Heart normal | 40 | 19.69 | 20.31 | 0 |
| Heart CHF | 38.5 | 19.4 | 19.1 | 0 |
| Kidney | 24.69 | 19.65 | 5.04 | 30.2903 |
| Skeletal Muscle | 40 | 21.35 | 18.65 | 0 |
| Adipose normal | 37.01 | 20.02 | 16.98 | 0 |
| Pancreas | 34.63 | 20.95 | 13.69 | 0.0759 |
| primary osteoblasts | 40 | 20.11 | 19.9 | 0 |
| Osteoclasts (diff) | 40 | 17.21 | 22.79 | 0 |
| Skin normal | 38.01 | 21.64 | 16.38 | 0 |
| Spinal cord normal | 35.54 | 20.49 | 15.05 | 0 |
| Brain Cortex normal | 40 | 22.41 | 17.59 | 0 |
| Brain Hypothalamus normal | 39.42 | 21.37 | 18.05 | 0 |
| Nerve | 40 | 21.5 | 18.5 | 0 |
| DRG (Dorsal Root Ganglion) | 39.77 | 21.41 | 18.36 | 0 |
| Breast normal | 31.99 | 20.46 | 11.53 | 0.3382 |
| Breast tumor | 32.71 | 20.48 | 12.23 | 0.2082 |
| Ovary normal | 39 | 19.89 | 19.11 | 0 |
| Ovary Tumor | 27.66 | 19.71 | 7.96 | 4.03 |
| Prostate Normal | 29.41 | 19.31 | 10.11 | 0.9049 |
| Prostate Tumor | 28.45 | 20.13 | 8.32 | 3.1184 |
| Salivary glands | 31.83 | 19.36 | 12.47 | 0.1769 |
| Colon normal | 33.91 | 18.02 | 15.88 | 0.0166 |
| Colon Tumor | 27.57 | 18.72 | 8.86 | 2.1522 |
| Lung normal | 29.16 | 17.77 | 11.39 | 0.3726 |
| Lung tumor | 26.45 | 19.84 | 6.6 | 10.3087 |
| Lung COPD | 28.7 | 18.16 | 10.53 | 0.6763 |
| Colon IBD | 26.57 | 17.29 | 9.29 | 1.603 |
| Liver normal | 37.22 | 19.73 | 17.49 | 0 |
| Liver fibrosis | 32.82 | 21.44 | 11.38 | 0.3752 |
| Spleen normal | 35.6 | 19.29 | 16.31 | 0 |
| Tonsil normal | 25.92 | 17.02 | 8.9 | 2.0933 |
| Lymph node normal | 33.03 | 18.23 | 14.8 | 0.0352 |
| Small intestine normal | 32.88 | 20.07 | 12.8 | 0.1402 |
| Skin-Decubitus | 36.37 | 20.41 | 15.96 | 0 |
| Synovium | 40 | 19.06 | 20.94 | 0 |
| BM-MNC | 39.2 | 18.06 | 21.14 | 0 |
| Activated PBMC | 40 | 18.58 | 21.42 | 0 |
| Neutrophils | 40 | 18.76 | 21.24 | 0 |
| Megakaryocytes | 40 | 18.55 | 21.45 | 0 |
| Erythroid | 38.83 | 21.41 | 17.42 | 0 |

As seen by these results, 38650, 28472, 5495, 81588 or 14354 molecules have been found to be overexpressed or underexpressed in some tumor or diseased cells. As such, 38650, 28472, 5495, 81588 or 14354 molecules may serve as specific and novel identifiers of such tumor cells. Further, modulators of the 38650, 28472, 5495, 81588 or 14354 molecules are useful for the treatment of diseases. Activators of the 38650, 28472, 5495, 81588 or 14354 molecules are useful for the treatment of cancer, preferably breast, ovary, prostate, colon, or lung cancer, blood vessel disorders, or a heart disorder where 38650, 28472, 5495, 81588 or 14354 is downregulated and useful as a diagnostic. Inhibitors of the 38650, 28472, 5495, 81588 or 14354 molecules are useful for the treatment of diseases or cancer, where 38650, 28472, 5495, 81588 or 14354 expression is upregulated, such as breast, ovary, prostate, colon, or lung cancer, blood vessel disorders, or a heart disorder and also useful as a diagnostic.

### Example 4

### Recombinant Expression of 38650, 28472. 5495, 65507, 81588 or 14354 in Bacterial Cells

In this example, 38650, 28472, 5495, 65507, 81588 or 14354 is expressed as a recombinant glutathione-S-transferase (GST) fusion polypeptide in E. coli and the fusion polypeptide is isolated and characterized. Specifically, 38650, 28472, 5495, 65507, 81588 or 14354 is fused to GST and this fusion polypeptide is expressed in E. coli, e.g., strain PEB199. Expression of the GST-38650, 28472, 5495, 65507, 81588 or 14354 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 5

### Expression of Recombinant 38650, 28472, 5495, 65507. 81588 or 14354 Protein in COS Cells

To express the 38650, 28472, 5495, 65507, 81588 or 14354 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 38650, 28472, 5495, 65507, 81588 or 14354 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 38650, 28472, 5495, 65507, 81588 or 14354 DNA sequence is amplified by PCR using two primers. The 5′ primer contains the restriction site of interest followed by approximately twenty nucleotides of the 38650, 28472, 5495, 65507, 81588 or 14354 coding sequence starting from the initiation codon; the 3′ end sequence contains complementary sequences to the other restriction site of interest, a translation stop codon, the HA tag or FLAG tag and the last 20 nucleotides of the 38650, 28472, 5495, 65507, 81588 or 14354 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the 38650, 28472, 5495, 65507, 81588 or 14354 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 38650, 28472, 5495, 65507, 81588 or 14354-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 38650, 28472, 5495, 65507, 81588 or 14354 polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or 35S-cysteine). cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 38650, 28472, 5495, 65507, 81588 or 14354 coding sequence is cloned directly into the polylinker of the pCDNA/Amp vector using the appropriate restriction sites. The resulting plasmid is transfected into COS cells in the manner described above, and the expression of the 38650, 28472, 5495, 65507, 81588 or 14354 polypeptide is detected by radiolabelling and immunoprecipitation using a 38650, 28472, 5495, 65507, 81588 or 14354 specific monoclonal antibody.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated 38650, 28472, 5495, 65507, 81588 or 14354 nucleic acid molecule selected from the group consisting of:
a) a nucleic acid molecule comprising a nucleotide sequence which is at least 60% identical to the nucleotide sequence of SEQ ID NO:1, 3, 4, 6, 7, 9, 10, 12, 13, 15, 16 or 18, or the nucleotide sequence of the DNA insert of the plasmid deposited with ATCC as Accession Number______;
b) a nucleic acid molecule comprising a fragment of at least 15 nucleotides of the nucleotide sequence of SEQ ID NO:1, 3, 4, 6, 7, 9, 10, 12, 13, 15, 16 or 18, or the nucleotide sequence of the DNA insert of the plasmid deposited with ATCC as Accession Number ______;
c) a nucleic acid molecule which encodes a polypeptide comprising the amino acid sequence of SEQ ID NO:2, 5, 8, 11, 14 or 17, or the amino acid sequence encoded by the cDNA insert of the plasmid deposited with the ATCC as Accession Number______;
d) a nucleic acid molecule which encodes a fragment of a polypeptide comprising the amino acid sequence of SEQ ID NO:2, 5, 8, 11, 14 or 17, or the amino acid sequence encoded by the cDNA insert of the plasmid deposited with the ATCC as Accession Number______, wherein the fragment comprises at least 15 contiguous amino acids of SEQ ID NO:2, 5, 8, 11, 14 or 17, or the amino acid sequence encoded by the cDNA insert of the plasmid deposited with the ATCC as Accession Number______;
e) a nucleic acid molecule which encodes a naturally occurring allelic variant of a polypeptide comprising the amino acid sequence of SEQ ID NO:2, 5, 8, 11, 14 or 17, or the amino acid sequence encoded by the cDNA insert of the plasmid deposited with the ATCC as Accession Number______, wherein the nucleic acid molecule hybridizes to a nucleic acid molecule comprising SEQ ID NO:1, 3, 4, 6, 7, 9, 10, 12, 13, 15, 16 or 18, or a complement thereof, under stringent conditions;
f) a nucleic acid molecule comprising the nucleotide sequence of SEQ ID NO:1, 3, 4, 6, 7, 9, 10, 12, 13, 15, 16 or 18, or the nucleotide sequence of the DNA insert of the plasmid deposited with ATCC as Accession Number______; and
g) a nucleic acid molecule which encodes a polypeptide comprising the amino acid sequence of SEQ ID NO:2, 5, 8, 11, 14 or 17, or the amino acid sequence encoded by the cDNA insert of the plasmid deposited with the ATCC as Accession Number______.

2. The isolated nucleic acid molecule of claim 1, which is the nucleotide sequence SEQ ID NO:1, 4, 7, 10, 13 or 16.

3. A host cell which contains the nucleic acid molecule of claim 1.

4. An isolated 38650, 28472, 5495, 65507, 81588 or 14354 polypeptide selected from the group consisting of:
a) a polypeptide which is encoded by a nucleic acid molecule comprising a nucleotide sequence which is at least 60% identical to a nucleic acid comprising the nucleotide sequence of SEQ ID NO:1, 3, 4, 6, 7, 9, 10, 12, 13, 15, 16 or 18, or the nucleotide sequence of the DNA insert of the plasmid deposited with ATCC as Accession Number ______, or a complement thereof,
b) a naturally occurring allelic variant of a polypeptide comprising the amino acid sequence of SEQ ID NO:2, 5, 8, 11, 14 or 17, or the amino acid sequence encoded by the cDNA insert of the plasmid deposited with the ATCC as Accession Number______, wherein the polypeptide is encoded by a nucleic acid molecule which hybridizes to a nucleic acid molecule comprising SEQ ID NO:1, 3, 4, 6, 7, 9, 10, 12, 13, 15, 16 or 18, or a complement thereof under stringent conditions;
c) a fragment of a polypeptide comprising the amino acid sequence of SEQ ID NO:2, 5, 8, 11, 14 or 17, or the amino acid sequence encoded by the cDNA insert of the plasmid deposited with the ATCC as Accession Number______, wherein the fragment comprises at least 15 contiguous amino acids of SEQ ID NO:2, 5, 8, 11, 14 or 17; and
d) the amino acid sequence of SEQ ID NO:2, 5, 8, 11, 14 or 17.

5. An antibody which selectively binds to a polypeptide of claim 4.

6. A method for producing a polypeptide selected from the group consisting of:
a) a polypeptide comprising the amino acid sequence of SEQ ID NO:2, 5, 8, 11, 14 or 17, or the amino acid sequence encoded by the cDNA insert of the plasmid deposited with the ATCC as Accession Number______;
b) a polypeptide comprising a fragment of the amino acid sequence of SEQ ID NO:2, 5, 8, 11, 14 or 17, or the amino acid sequence encoded by the cDNA insert of the plasmid deposited with the ATCC as Accession Number______, wherein the fragment comprises at least 15 contiguous amino acids of SEQ ID NO:2, 5, 8, 11, 14 or 17, or the amino acid sequence encoded by the cDNA insert of the plasmid deposited with the ATCC as Accession Number______;
c) a naturally occurring allelic variant of a polypeptide comprising the amino acid sequence of SEQ ID NO:2, 5, 8, 11, 14 or 17, or the amino acid sequence encoded by the cDNA insert of the plasmid deposited with the ATCC as Accession Number______, wherein the polypeptide is encoded by a nucleic acid molecule which hybridizes to a nucleic acid molecule comprising SEQ ID NO:1, 3, 4, 6, 7, 9, 10, 12, 13, 15, 16 or 18; and
d) the amino acid sequence of SEQ ID NO:2, 5, 8, 11, 14 or 17; comprising culturing the host cell of claim 3 under conditions in which the nucleic acid molecule is expressed.

7. A method for detecting the presence of a nucleic acid molecule of claim 1 or a polypeptide encoded by the nucleic acid molecule in a sample, comprising:
a) contacting the sample with a compound which selectively hybridizes to the nucleic acid molecule of claim 1 or binds to the polypeptide encoded by the nucleic acid molecule; and
b) determining whether the compound hybridizes to the nucleic acid or binds to the polypeptide in the sample.

8. A kit comprising a compound which selectively hybridizes to a nucleic acid molecule of claim 1 or binds to a polypeptide encoded by the nucleic acid molecule and instructions for use.

9. A method for identifying a compound which binds to a polypeptide or modulates the activity of the polypeptide of claim 4 comprising the steps of:
a) contacting a polypeptide, or a cell expressing a polypeptide of claim 4 with a test compound; and
b) determining whether the polypeptide binds to the test compound or determining the effect of the test compound on the activity of the polypeptide.

10. A method for modulating the activity of a polypeptide of claim 4 comprising contacting the polypeptide or a cell expressing the polypeptide with a compound which binds to the polypeptide in a sufficient concentration to modulate the activity of the polypeptide.

11. A method of identifying a nucleic acid molecule associated with a disorder comprising:
a) contacting a sample from a subject with or at risk of developing a disorder comprising nucleic acid molecules with a hybridization probe comprising at least 25 contiguous nucleotides of SEQ ID NO:1, 4, 7, 10, 13 or 16 defined in claim 2; and
b) detecting the presence of a nucleic acid molecule in the sample that hybridizes to the probe, thereby identifying a nucleic acid molecule associated with a disorder.

12. A method of identifying a nucleic acid associated with a disorder comprising:
a) contacting a sample from a subject having a disorder or at risk of developing a disorder comprising nucleic acid molecules with a first and a second amplification primer, the first primer comprising at least 25 contiguous nucleotides of SEQ ID NO:1, 4, 7, 10, 13 or 16 defined in claim 2 and the second primer comprising at least 25 contiguous nucleotides from the complement of SEQ ID NO:1, 4, 7, 10, 13 or 16;
b) incubating the sample under conditions that allow nucleic acid amplification; and
c) detecting the presence of a nucleic acid molecule in the sample that is amplified, thereby identifying the nucleic acid molecule associated with a disorder.

13. A method of identifying a polypeptide associated with a disorder comprising:
a) contacting a sample comprising polypeptides with a 38650, 28472, 5495, 65507, 81588 or 14354 binding partner of the 38650, 28472, 5495, 65507, 81588 or 14354 polypeptide defined in claim 4; and
b) detecting the presence of a polypeptide in the sample that binds to the 38650, 28472, 5495, 65507, 81588 or 14354 binding partner, thereby identifying the polypeptide associated with a disorder.

14. A method of identifying a subject having a disorder or at risk for developing a disorder comprising:
a) contacting a sample obtained from the subject comprising nucleic acid molecules with a hybridization probe comprising at least 25 contiguous nucleotides of SEQ ID NO:1, 4, 7, 10, 13 or 16 defined in claim 2; and
b) detecting the presence of a nucleic acid molecule in the sample that hybridizes to the probe, thereby identifying a subject having a disorder or at risk for developing a disorder.

15. A method of identifying a subject having a disorder or at risk for developing a disorder comprising:
a) contacting a sample obtained from the subject comprising nucleic acid molecules with a first and a second amplification primer, the first primer comprising at least 25 contiguous nucleotides of SEQ ID NO:1, 4, 7, 10, 13 or 16 defined in claim 2 and the second primer comprising at least 25 contiguous nucleotides from the complement of SEQ ID NO:1, 4, 7, 10, 13 or 16;
b) incubating the sample under conditions that allow nucleic acid amplification; and
c) detecting the presence of a nucleic acid molecule in the sample that is amplified, thereby identifying a subject having a disorder or at risk for developing a disorder.

16. A method of identifying a subject having a disorder or at risk for developing a disorder comprising:
a) contacting a sample obtained from the subject comprising polypeptides with a 38650, 28472, 5495, 65507, 81588 or 14354 binding partner of the 38650, 28472, 5495, 65507, 81588 or 14354 polypeptide defined in claim 4; and
b) detecting the presence of a polypeptide in the sample that binds to the 38650, 28472, 5495, 65507, 81588 or 14354 binding partner, thereby identifying a subject having a disorder or at risk for developing a disorder.

17. A method for identifying a compound capable of treating a disorder characterized by aberrant 38650, 28472, 5495, 65507, 81588 or 14354 nucleic acid expression or 38650, 28472, 5495, 65507, 81588 or 14354 polypeptide activity comprising assaying the ability of the compound to modulate 38650, 28472, 5495, 65507, 81588 or 14354 nucleic acid expression or 38650, 28472, 5495, 65507, 81588 or 14354 polypeptide activity, thereby identifying a compound capable of treating a disorder characterized by aberrant 38650, 28472, 5495, 65507, 81588 or 14354 nucleic acid expression or 38650, 28472, 5495, 65507, 81588 or 14354 polypeptide activity.

18. A method for treating a subject having a disorder or at risk of developing a disorder comprising administering to the subject a 38650, 28472, 5495, 65507, 81588 or 14354 modulator of the nucleic acid molecule defined in claim 1 or the polypeptide encoded by the nucleic acid molecule or contacting a cell with a 38650, 28472, 5495, 65507, 81588 or 14354 modulator.

19. The method of claim 18, wherein the 38650, 28472, 5495, 65507, 81588 or 14354 modulator is
a) a small molecule;
b) peptide;
c) phosphopeptide;
d) anti-38650, 28472, 5495, 65507, 81588 or 14354 antibody;
e) a 38650, 28472, 5495, 65507, 81588 or 14354 polypeptide comprising the amino acid sequence of SEQ ID NO:2, 5, 8, 11, 14 or 17, or a fragment thereof;
f) a 38650, 28472, 5495, 65507, 81588 or 14354 polypeptide comprising an amino acid sequence which is at least 90 percent identical to the amino acid sequence of SEQ ID NO:2, 5, 8, 11, 14 or 17, wherein the percent identity is calculated using the ALIGN program for comparing amino acid sequences, a PAM120 weight residue table, a gap length penalty of 12, and a gap penalty of 4; or
g) an isolated naturally occurring allelic variant of a polypeptide consisting of the amino acid sequence of SEQ ID NO:2, 5, 8, 11, 14 or 17, wherein the polypeptide is encoded by a nucleic acid molecule which hybridizes to a complement of a nucleic acid molecule consisting of SEQ ID NO:1, 4, 7, 10, 13 or 16 at 6×SSC at 45° C, followed by one or more washes in 0.2×SSC, 0.1% SDS at 65° C.

20. The method of claim 18, wherein the 38650, 28472, 5495, 65507, 81588 or 14354 modulator is
a) an antisense 38650, 28472, 5495, 65507, 81588 or 14354 nucleic acid molecule;
b) is a ribozyme;
c) the nucleotide sequence of SEQ ID NO:1, 4, 7, 10, 13 or 16, or a fragment thereof;
d) a nucleic acid molecule encoding a polypeptide comprising an amino acid sequence which is at least 90 percent identical to the amino acid sequence of SEQ ID NO:2, 5, 8, 11, 14 or 17, wherein the percent identity is calculated using the ALIGN program for comparing amino acid sequences, a PAM120 weight residue table, a gap length penalty of 12, and a gap penalty of 4;
e) a nucleic acid molecule encoding a naturally occurring allelic variant of a polypeptide comprising the amino acid sequence of SEQ ID NO:2, 5, 8, 11, 14 or 17, wherein the nucleic acid molecule which hybridizes to a complement of a nucleic acid molecule consisting of SEQ ID NO:1 at 6×SSC at 45° C., followed by one or more washes in 0.2×SSC, 0.1% SDS at 65° C.; or
f) a gene therapy vector.

21. A method for evaluating the efficacy of a treatment of a disorder, in a subject, comprising:
treating a subject with a protocol under evaluation;
assessing the expression level of a 38650, 28472, 5495, 65507, 81588 or 14354 nucleic acid molecule defined in claim 1 or 38650, 28472, 5495, 65507, 81588 or 14354 polypeptide encoded by the 38650, 28472, 5495, 65507, 81588 or 14354 nucleic acid molecule,
wherein a change in the expression level of 38650, 28472, 5495, 65507, 81588 or 14354 nucleic acid or 38650, 28472, 5495, 65507, 81588 or 14354 polypeptide after the treatment, relative to the level before the treatment, is indicative of the efficacy of the treatment of a disorder.

22. A method of diagnosing a disorder in a subject, comprising: evaluating the expression or activity of a 38650, 28472, 5495, 65507, 81588 or 14354 nucleic acid molecule defined in claim 1 or a 38650, 28472, 5495, 65507, 81588 or 14354 polypeptide encoded by the 38650, 28472, 5495, 65507, 81588 or 14354 nucleic acid molecule, such that a difference in the level of 38650, 28472, 5495, 65507, 81588 or 14354 nucleic acid or 38650, 28472, 5495, 65507, 81588 or 14354 polypeptide relative to a normal subject or a cohort of normal subjects is indicative of a disorder.

23. The method defined in claim 18, wherein the disorder is cancer or aberrant cellular proliferation and/or differentiation, immune disorders, heart disorders, cardiovascular disorders, including endothelial cell disorders, hematopoietic disorders, blood vessel disorders, brain disorders, pain and metabolic disorders, liver disorders and platelet disorders.

24. The method defined in claim 23, wherein the cancer or aberrant cellular proliferation and/or differentiation is breast, ovarian, prostate, colon, or lung cancer.
